(19) United States
(12) Patent Application Publication
(10) Pub. No.: US 2024/0167022 A1
(43) Pub. Date: May 23, 2024

(54) TEMPLATE DIRECTED IMMUNOMODULATION FOR CANCER THERAPY

(71) Applicant: Transcode Therapeutics, Inc., Boston, MA (US)

(72) Inventors: Robert Michael Dudley, Boston, MA (US); Qiyong Peter Liu, Boston, MA (US); Subrata Kumar Ghosh, Boston, MA (US)

(21) Appl. No.: 18/270,361

(22) PCT Filed: Dec. 29, 2021
(86) PCT No.: PCT/US2021/065580

§ 371 Date: Jun. 29, 2023

Related U.S. Application Data
(60) Provisional application No. 63/132,315, filed on Dec. 30, 2020.

(51) Int. Cl. C12N 15/11; A61K 9/51; A61K 39/395; A61K 45/06; A61P 35/00

(52) U.S. Cl. CPC C12N 15/11; A61K 9/5115; A61K 9/5161; A61K 39/3955; A61K 45/06; A61P 35/00; C12N 2310/14; C12N 2310/141; C12N 2310/321; C12N 2310/334; C12N 2310/335

(57) ABSTRACT

Described herein are compositions and methods for treating cancer comprising single-stranded 5′ uncapped triphosphate or biphosphate modified RNA oligonucleotides complementary to a miRNA which is highly expressed in a tumor microenvironment in comparison to a non-tumor environment.

### CROSS-REFERENCE TO RELATED APPLICATION

This application claims the benefit of priority to the U.S. Provisional Application No. 63/132,315, filed on Dec. 30, 2020. The specification of the foregoing application is hereby incorporated by reference in its entirety.

### BACKGROUND OF THE INVENTION

Cancer represents a continuing and significant threat to global human health. Harnessing novel mechanisms for treating cancers represents a promising means of delivering therapeutics that meet the ongoing and urgent need for effective cancer treatment. Recent studies have shown that systemic delivery of a synthetic RIG-I (retinoic acid-inducible gene I) agonist inhibits tumor growth. RIG-I senses short double-stranded RNAs with an uncapped 5-triphosphate moiety, a common motif typically found in viral RNAs. RIG-I is expressed in numerous cell types, including tumor cells, and serves as a promising target for cancer therapy. It is therefore the object of the present disclosure to provide compositions and methods for selectively activating RIG-I in a tumor microenvironment in order to treat cancers. A therapeutic methodology harnessing endogenous miRNAs as a means for activating RIG-I provides a highly promising approach to target the tumor microenvironment and treat various associated cancers.

The compositions and methods of the present disclosure provide methods for selectively activating RIG-I in a tumor microenvironment utilizing single-stranded 5′ uncapped triphosphate or biphosphate modified RNA oligonucleotides complementary to miRNAs that are highly expressed in a tumor microenvironment in comparison to a non-tumor environment.

### SUMMARY OF THE INVENTION

In certain aspects, the disclosure relates to methods for treating cancer comprising administering to a subject a therapeutically effective amount of a single-stranded 5′ uncapped triphosphate or biphosphate modified RNA oligonucleotide, wherein said oligonucleotide is complementary to a miRNA, which is highly expressed in a tumor or tumor microenvironment in comparison to a non-tumor or non-tumor microenvironment.

In certain aspects, the disclosure relates to methods for selectively activating RIG-I in a tumor or tumor microenvironment comprising administering to a subject a therapeutically effective amount of single-stranded 5′ uncapped triphosphate or biphosphate modified RNA oligonucleotide, wherein said single-stranded 5′ uncapped triphosphate or biphosphate modified RNA oligonucleotide comprises a sequence which is complementary to a miRNA expressed in the tumor or tumor microenvironment, wherein the RIG-I is selectively activated in the tumor or tumor microenvironment expressing the miRNA.

In some embodiments, the miRNA is selected from the group consisting of miR10b, miR17, miR18a, miR18b, miR19b, miR21, miR26a, miR29a, miR92a-1, miR92a-2, miR155, miR210, and miR221. In some embodiments, the single-stranded 5′ uncapped triphosphate or biphosphate modified RNA oligonucleotide forms a duplex with the miRNA. In some embodiments, the miRNA is oncogenic miRNA. In some embodiments, the miRNA is a tumor-associated miRNA. In some embodiments, the duplex is not cleaved by AGO2. In some embodiments, the duplex activates RIG-I. In some embodiments, the RIG-I activation is at least 5%, 10%, 15% or 20% greater than activation by a corresponding unmodified monophosphate RNA oligonucleotide. In some embodiments, the RIG-I activation elicits a tumor-specific immune response. In some embodiments, the tumor-specific immune response comprises release of type I IFNs, DAMPs (danger-associated molecular patterns), and/or tumor antigens. In some embodiments, the method induces immunological memory against said tumor or tumor microenvironment.

In some embodiments, the cancer is a solid tumor. In some embodiments, the solid tumor is selected from the group consisting of sarcomas, carcinomas, and lymphomas. In some embodiments, the cancer is selected from the group consisting of bladder, blood, bone, brain, breast, colon, cervix, kidney, esophagus, liver, lung, thyroid, skin, ovarian, pancreatic, prostate, rectal, stomach, uterine cancer, glioblastoma, or head and neck cancer. In some embodiments, the modified RNA oligonucleotide does not comprise any other modifications.

In some embodiments, the modified RNA oligonucleotide comprises at least 2 different modified RNA oligonucleotides. In some embodiments, the modified RNA oligonucleotide comprises at least 3 different modified RNA oligonucleotides. In some embodiments, the modified RNA oligonucleotide comprises at least 4 different modified RNA oligonucleotides. In some embodiments, the modified RNA oligonucleotide comprises at least 5 different modified RNA oligonucleotides. In some embodiments, the modified RNA oligonucleotide comprises up to 40 different modified RNA oligonucleotides.

In some embodiments, the modified RNA oligonucleotide further comprises a 2′-fluoro (2′-F) ribose modification. In some embodiments, the 2′-F ribose modification is present at the 10^{th} or 11^{th} nucleotide from the 5′-terminus of the modified RNA oligonucleotide. In some embodiments, the modified RNA oligonucleotide does not comprise a 2′-O-methyl (2′-OMe) ribose modification. In some embodiments, the modified RNA oligonucleotide does not comprise a N-6-methyladenosine (m6A) modification. In some embodiments, the modified RNA oligonucleotide does not comprise a pseudouridine (Ψ). In some embodiments, the modified RNA oligonucleotide does not comprise a N-1-methylpseudouridine (mΨ) modification. In some embodiments, the modified RNA oligonucleotide does not comprise a 5-methyl-cytidine (5mC) modification. In some embodiments, the modified RNA oligonucleotide does not comprise a 5-hydroxymethyl-cytidine (5hmC) modification. In some embodiments, the modified RNA oligonucleotide does not comprise a 5-methoxycytidine (5moC) modification.

In some embodiments, the modified RNA oligonucleotide comprises a sequence which is at least 19 nucleotides in length. In some embodiments, the modified RNA oligonucleotide comprises a sequence which is between 15 and 30 nucleotides in length. In some embodiments, the modified RNA oligonucleotide comprises a sequence which is between 16 and 27 nucleotides in length. In some embodiments, the modified RNA oligonucleotide is fully complementary to the miRNA. In some embodiments, the modified RNA oligonucleotide competes with endogenous mRNA to bind the miRNA. In some embodiments, the duplex comprises between 0 and 5 mismatched base pairs.

In some embodiments, the method comprises administering a modified RNA oligonucleotide having the nucleic acid sequence of SEQ ID NO: 6. In some embodiments, the nucleic acid of SEQ ID NO: 6 is complementary to miR-21. In some embodiments, the cancer is selected from the group consisting of cancer of the breast, ovary, cervix, colon, lung, liver, brain, esophagus, prostate, pancreas, and thyroid. In some embodiments, the method comprises administering a modified RNA oligonucleotide having the nucleic acid sequence of SEQ ID NO: 1. In some embodiments, the nucleic acid of SEQ ID NO: 1 is complementary to miR-10b. In some embodiments, the cancer is non-small cell lung cancer or cervical cancer. In some embodiments, the cancer is metastatic cancer. In some embodiments, the cytosine and uracil are present at the AGO2 cleavage site. In some embodiments, the metastatic cancer is localized in breast, lymph nodes, lung, bone, brain, liver, ovary, peritoneum, muscle tissue, pancreas, prostate, esophagus, colon, rectum, stomach, nasopharyngeal or skin. In some embodiments, the treatment with the modified RNA oligonucleotide is a monotherapy. In some embodiments, the modified RNA oligonucleotide is administered by intravenous administration, subcutaneous, intraarterial, intramuscular, intraperitoneal, or local administration. In some embodiments, the modified RNA oligonucleotide is administered at a dose of about 0.2 mg/kg to about 200 mg/kg. In some embodiments, the modified RNA oligonucleotide is administered at a dose of about 0.2 mg/kg to about 2.0 mg/kg. In some embodiments, the modified RNA oligonucleotide is administered at a dose of about 1.0 mg/kg to about 10.0 mg/kg.

In certain aspects, the disclosure relates to methods for treating cancer comprising administering to a subject a therapeutically effective amount of a magnetic nanoparticle comprising: ferric chloride, ferrous chloride, or a combination thereof; a dextran coating; and a single-stranded 5′ uncapped triphosphate or biphosphate modified RNA oligonucleotide, wherein said oligonucleotide is complementary to a miRNA, which is highly expressed in a tumor or tumor microenvironment in comparison to a non-tumor or non-tumor microenvironment. In some embodiments, the magnetic nanoparticle has a non-linearity index ranging from about 6 to about 40. In some embodiments, the magnetic nanoparticle has a non-linearity index ranging from about 8 to about 14. In some embodiments, the magnetic nanoparticle comprises about 0.54 g of ferric chloride and about 0.2 g of ferrous chloride. In some embodiments, the miRNA is selected from the group consisting of miR10b, miR17, miR18a, miR18b, miR19b, miR21, miR26a, miR29a, miR92a-1, miR92a-2, miR155, miR210, and miR221. In some embodiments, the miRNA is oncogenic miRNA. In some embodiments, the miRNA is a tumor-associated miRNA.

In some embodiments, the method further comprises administering supportive or adjunctive therapy. In some embodiments, the adjunctive therapy comprises radiotherapy, cryotherapy, and ultrasound therapy.

In some embodiments, the method comprises administering additional therapeutic agents. In some embodiments, the additional therapeutic agent comprises a miRNA. In some embodiments, the miRNA is complementary to the modified RNA oligonucleotide. In some embodiments, the additional therapeutic agent is selected from the group consisting of a targeted therapy, chemotherapeutic agent, immunotherapeutic agent, an immunogenic cell death inducer (ICDi), and an siRNA therapy. In some embodiments, the method further comprises surgery. In some embodiments, the chemotherapeutic agent is selected from the group consisting of cyclophosphamide, mechlorethamine, chlorambucil, melphalan, daunorubicin, doxorubicin, epirubicin, idarubicin, mitoxantrone, valrubicin, paclitaxel, docetaxel, etoposide, teniposide, tafluposide, azacitidine, azathioprine, capecitabine, cytarabine, doxifluridine, fluorouracil, gemcitabine, mercaptopurine, methotrexate, tioguanine, bleomycin, carboplatin, cisplatin, oxaliplatin, all-trans retinoic acid, vinblastine, vincristine, vindesine, vinorelbine, and bevacizumab. In some embodiments, the targeted therapy is selected from the group consisting of trastuzumab, gilotrif, proleukin, alectinib, campath, atezolizumab, avelumab, axitinib, belimumab, belinostat, bevacizumab, velcade, canakinumab, ceritinib, cetuximab, crizotinib, dabrafenib, daratumumab, dasatinib, denosumab, elotuzumab, enasidenib, erlotinib, gefitinib, ibrutinib, zydelig, imatinib, lenvatinib, midostaurin, necitumumab, niraparib, obinutuzumab, osimertinib, panitumumab, regorafenib, rituximab, ruxolitinib, sorafenib, tocilizumab, and trastuzumab. In some embodiments, the immunotherapeutic agent is an immune checkpoint inhibitor. In some embodiments, the immune checkpoint inhibitor is selected from the group consisting of pembrolizumab (Keytruda®), nivolumab (Opdivo®), atezolizumab (Tecentriq®), ipilimumab (Yervoy®), avelumab (Bavencio®) and durvalumab (Imfinzi®). In some embodiments, the adjunctive therapy induces expression of the miRNA. In some embodiments, the additional therapeutic agent induces expression of the miRNA. In some embodiments, the ICDi is selected from the group consisting of Daunorubicin, Docetaxel, Doxorubicin. Mitoxanthrone, Oxaliplatin, and Paclitaxel. In some embodiments, the siRNA therapy targets PD-L1, CTLA-4, TGF-β, and/or VEGF. In some embodiments, the supportive or adjunctive therapy is administered prior, concurrently, or after administration of the modified RNA oligonucleotide.

In certain aspects, the disclosure relates to compositions comprising a single-stranded 5′ uncapped triphosphate or biphosphate modified RNA oligonucleotide, wherein said oligonucleotide is complementary to a miRNA, which is highly expressed in tumor tissue in comparison to non-tumor tissue. In some embodiments, the miRNA is selected from the group consisting of miR10b, miR17, miR18a, miR18b, miR19b, miR21, miR26a, miR29a, miR92a-1, miR92a-2, miR155, miR210, and miR221. In some embodiments, the modified RNA oligonucleotide is capable of forming a duplex with the said miRNA. In some embodiments, the duplex is not cleaved by AGO2. In some embodiments, the duplex activates RIG-I. In some embodiments, the RIG-I activation is at least 5%, 10%, 15% or 20% greater than activation by a corresponding unmodified monophosphate RNA oligonucleotide. In some embodiments, the RIG-I activation elicits a tumor-specific immune response. In some embodiments, the tumor-specific immune response comprises release of type I IFNs, DAMPs (danger-associated molecular patterns), and/or tumor antigens.

In some embodiments, the modified RNA oligonucleotide does not comprise any other modifications. In some embodiments, the modified RNA oligonucleotide further comprises a 2′-fluoro (2′-F) ribose modification. In some embodiments, the modified RNA oligonucleotide does not comprise a 2′-O-methyl (2′-OMe) ribose modification. In some embodiments, the modified RNA oligonucleotide does not comprise a N-6-methyladenosine (m6A) modification. In some embodiments, the modified RNA oligonucleotide does not comprise a pseudouridine (Ψ). In some embodiments, the modified RNA oligonucleotide does not comprise a N-1-methylpseudouridine (mΨ) modification. In some embodiments, the modified RNA oligonucleotide does not comprise a 5-methyl-cytidine (5mC) modification. In some embodiments, the modified RNA oligonucleotide does not comprise a 5-hydroxymethyl-cytidine (5hmC) modification. In some embodiments, the modified RNA oligonucleotide does not comprise a 5-methoxycytidine (5moC) modification. In some embodiments, the modified RNA oligonucleotide is fully complementary to the miRNA. In some embodiments, the modified RNA oligonucleotide competes with endogenous mRNA to bind the miRNA. In some embodiments, the duplex comprises between 0 and 5 mismatched base pairs. In some embodiments, the modified RNA oligonucleotide comprises a nucleic acid sequence of any one of SEQ ID NOs: 1-13.

In some embodiments, the modified oligonucleotide is further linked to a nanoparticle. In some embodiments, the nanoparticle is a magnetic nanoparticle. In some embodiments, the magnetic nanoparticle is coated with a polymer coating. In some embodiments, the polymer coating is dextran. In some embodiments, the magnetic nanoparticle comprises iron oxide; and a dextran coating functionalized with one or more amine groups, wherein the number of the one or more amine groups ranges from about 5 to about 1000. In some embodiments, the iron content of the magnetic nanoparticle comprises about 50% weight (wt) to about 100% wt of iron (III) and about 0% wt to about 50% wt of iron (II). In some embodiments, the magnetic nanoparticle comprises from about 5 to about 150 amino groups. In some embodiments, the magnetic nanoparticle comprises one or more such modified RNA oligonucleotides.

In certain aspects, the disclosure relates to compositions comprising a magnetic nanoparticle comprising: ferric chloride, ferrous chloride, or a combination thereof; a dextran coating; and a single-stranded 5′ uncapped triphosphate or biphosphate modified RNA oligonucleotide, wherein said oligonucleotide is complementary to a miRNA, which is highly expressed in a tumor or tumor microenvironment in comparison to a non-tumor or non-tumor microenvironment. In some embodiments, the magnetic nanoparticle has a non-linearity index ranging from about 6 to about 40. In some embodiments, the magnetic nanoparticle has a non-linearity index ranging from about 8 to about 14. In some embodiments, the magnetic nanoparticle comprises about 0.54 g of ferric chloride and about 0.2 g of ferrous chloride. In some embodiments, the miRNA is selected from the group consisting of miR10b, miR17, miR18a, miR18b, miR19b, miR21, miR26a, miR29a, miR92a-1, miR92a-2, miR155, miR210, and miR221. In some embodiments, the miRNA is oncogenic miRNA. In some embodiments, the miRNA is a tumor-associated miRNA.

In some embodiments, the magnetic nanoparticle comprises two or more modified RNA oligonucleotides. In some embodiments, the two or more modified RNA oligonucleotides are complementary to different miRNAs. In some embodiments, the two or more modified RNA oligonucleotides are complementary to the same miRNA.

In certain aspects, the disclosure relates to a pharmaceutical composition comprising a modified RNA oligonucleotide or a magnetic nanoparticle as disclosed herein. In some embodiments, the pharmaceutical composition further comprises a delivery agent. In some embodiments, the delivery agent is selected from the group consisting of a micelle, lipid nanoparticle (LNP), spherical nucleic acid (SNA), extracellular vesicle, synthetic vesicle, exosome, lipidoid, liposome, and lipoplex. In some embodiments, the liposome is formed from a lipid bilayer. In some embodiments, the lipid bilayer comprises one or more phospholipids selected from the group consisting of phosphate lipids, phosphoglycerol lipids, phosphocholine lipids, and phosphoethanolamine lipids. In some embodiments, the phospholipids are PEGylated. In some embodiments, the delivery agent is a liposome or lipid nanoparticle. In some embodiments, the liposome or lipid nanoparticle further delivers an additional therapeutic agent. In some embodiments, the additional therapeutic agent is an ICDi (e.g., Daunorubicin, Docetaxel, Doxorubicin, Mitoxanthrone, Oxaliplatin, and Paclitaxel). In some embodiments, the additional therapeutic agent is an siRNA (e.g., an siRNA targeting a gene associated with cancer). In some embodiments, the additional therapeutic agent is a chemotherapeutic agent. In some embodiments, the pharmaceutical composition further comprises at least one additional modified RNA oligonucleotide. In some embodiments, the modified RNA oligonucleotide is administered at a dose of about 0.2 mg/kg to about 200 mg/kg. In some embodiments, the modified RNA oligonucleotide is administered at a dose of about 0.2 mg/kg to about 2.0 mg/kg. In some embodiments, the modified RNA oligonucleotide is administered at a dose of about 1.0 mg/kg to about 10.0 mg/kg.

In some embodiments, provided herein are single stranded antisense RNAs comprising a sequence that is complementary to a miRNA or mRNA, with a 5′ biphosphate (5′pp anti-miRNA or -mRNA) or 5′ triphosphate modification (5′ppp anti-miRNA or -mRNA), preferably wherein the miRNA or mRNA is listed in Table 1, Table 2, or Table 3, respectively. Also provided are RIG-I agonists comprising a 5′ biphosphate (5′pp) or 5′ triphosphate (5′ppp) modified RNA of at least 10-nucleotides in length that are complementary to an endogenous (preferably tumor-specific) RNA sequence. In some embodiments, the nucleic acid comprises at least one modified nucleotide. In some embodiments, the at least one modified nucleotide is a locked nucleotide.

In some embodiments, provided herein are compositions and methods comprising 5′pp or 5′ppp anti-miRNAs/mRNAs for eliciting an immune response to specific RNAs, e.g., endogenous RNA sequences, e.g., to treat and reduce risk of developing cancer.

In some embodiments, the single-stranded antisense RNA is linked to a nanoparticle, wherein said nanoparticle: has a diameter of between 10 nm to 30 nm; and comprises a polymer coating.

In some embodiments, the single-stranded antisense RNA is linked to a nanoparticle, wherein said nanoparticle: has a diameter of between 10 nm to 30 nm; and comprises a polymer coating. In some embodiments, the polymer coating comprises dextran.

In some embodiments, the single-stranded antisense RNA is covalently linked at 3′ end to the nanoparticle through a chemical moiety comprising a disulfide bond or a thioether bond. In some embodiments, the nanoparticle is magnetic.

Also provided herein are pharmaceutical compositions comprising a single-stranded antisense RNA as described herein. Additionally, provided herein are methods for treating or reducing the risk of developing cancer in a subject. The methods include administering a therapeutically effective amount of a single-stranded antisense RNA as described herein to a subject having a cancer or at risk of developing a cancer. In some embodiments, the cancer is selected from the group consisting of bladder, blood, bone, brain, breast, colon, kidney, liver, lung, skin, ovarian, pancreatic, prostate, rectal, stomach, thyroid, and uterine cancer.

In some embodiments, the administering results in a decrease or stabilization of tumor size, or a decrease in the rate of metastatic tumor growth in a lymph node in the subject. In some embodiments, the single-stranded antisense RNA is administered in two or more doses to the subject. In some embodiments, the single-stranded antisense RNA is administered to the subject at least once a week. In some embodiments, the single-stranded antisense RNA is administered to the subject by intravenous, subcutaneous, intraarterial, intramuscular, or intraperitoneal administration. In some embodiments, the subject is further administered a chemotherapeutic agent.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the invention will be apparent from the following detailed description and figures, and from the claims.

### BRIEF DESCRIPTION OF THE DRAWINGS

FIG. 1. is a schematic illustration of the delivery of a 5′triphosphorylated antisense tsRNA, delivered to tumors and metastases using the nanoparticle delivery system described herein. The antisense tsRNA and tumor specific tsRNA through hybridization produce a 5′ppp-dsRNA, a potent RIG-I agonist. Activation of the RIG-I signaling pathway leads to a type I IFN-driven immune response specific to the tumor microenvironment. This immune response is characterized by activation of dendritic cells (DCs), natural killer cells (NKs), and macrophages. This process is accompanied by effective tumor antigen presentation by the activated DCs and macrophages and T cell maturation, activation and tumor cell killing. Concomitantly, regulatory T cells (Tregs) are inhibited reducing their immunosuppressive action against the anti-tumor immune response. Importantly, a memory T cell subpopulation is generated that triggers complete immune rejection of the tumor as foreign upon rechallenge. Combined, these processes lead to full remission and resistance to cancer recurrence.

FIG. 2 provides summary data demonstrating the capacity of ss-ppp-miRNA-21 to induce RIG-I activation in the human RIG-I luciferase reporter cell line, HEK-Lucia™ RIG-1. High expression of RIG-I in the cells was confirmed using Western Blot (FIG. 2A). A highly significant enhancement of luciferase activity was observed in the RIG-I overexpressing cells, as compared to the null cells (FIG. 2B). 2 μg/mL, 4 μg/mL, and 8 μg/mL dose levels of ss-ppp-miRNA-21 were evaluated in HEK-Lucia™ RIG-I. Significant RIG-I activation was observed at all three dose levels of ss-ppp-miRNA-21 tested (FIG. 2C). A dose-dependent caspase 3/7 activation was observed that was more pronounced in the presence of a 5′-ppp (FIG. 2D). A dose-dependent reduction in tumor cell viability was also observed when using the ss-ppp-miRNA-21 RIG-I agonist (FIG. 2E).

FIG. 3 provides summary data demonstrating induction of RIG-I signaling by the ss-ppp-miRNA-21 agonist in HEK-Lucia™ RIG-I cells transiently transfected with increasing concentrations of a synthetic mature miRNA-21 mimic. Cells were transfected with 0 ng/mL, 0.3 ng/mL, 3 ng/mL, 30 ng/mL, and 300 ng/mL concentrations of the synthetic mature miRNA-21 mimic; a highly significant induction of RIG-I signaling by the ss-ppp-miRNA-21 agonist was observed in cells transfected with 30 and 300 ng/ml of the synthetic mature miRNA-21 mimic; 5′-ppp-deficient ss-miRNA-21 failed to cause detectable RIG-I activation (FIG. 3A). Analysis of the dose-dependence of RIG-I activation as a function of miRNA-21 mimic concentration determined an EC50 of 83.4 ng/ml of miRNA-21 mimic when using ss-ppp-miRNA-21; by contrast, the calculated EC50 when using the 5′-ppp-deficient ss-miRNA-21 was 357.9 ng/ml (FIG. 3B). Treatment of B16-F10 murine melanoma cells with increasing concentrations of the RIG-I agonist caused a dose-dependent increase in IFN-β secretion; in contrast, a commercially available ds-ppp-RNA agonist failed to stimulate IFN-β secretion (FIG. 3C). Caspase 3/7 activation as a function of miRNA-21 mimic concentration was measured in B16-F10 muring melanoma cells; a dose-dependent increase in caspase 3/7 activation was observed, and the effect was significantly higher in cells treated with ss-ppp-miRNA-21 as compared to the 5′-ppp-deficient ss-miRNA-21, and comparable to the ds-ppp-RNA positive control (FIG. 3D). FIG. 3E is a western blot in which cells transfected with miR-21 and treated with ss-ppp-miRNA-21 exhibited a dramatic upregulation of RIG-I that exceeded the levels seen with the ds-ppp-RNA positive control oligonucleotide. FIG. 3F is a western blot which demonstrates the increased reactivity in cells transfected with miR-21 and treated with ss-ppp-miRNA-21 was not associated with increased expression of p65, indicating that the increase in reactivity specifically reflected target phosphorylation.

### DETAILED DESCRIPTION

### 1. Overview

miRNAs in Cancer

Small RNAs, such as miRNAs, exert their regulatory functions from within ribonucleoprotein complexes termed RISCs (RNA-induced silencing complexes). The core subunit of RISC is a small RNA bound to a member of the Argonaute family of proteins. Argonaute uses the small RNA as a guide to identify complementary target transcripts for silencing through a variety of mechanisms. MiRNAs are generally captured by the human Argonaute 2 protein (AGO2) and are capable of regulating gene expression by base-pairing to complementary mRNA targets while associated with AGO2. The miRNA captured by AGO2 serves as a guide RNA to accept and hybridize with complementary RNA targets, forming a double-stranded RNA duplex. It has been shown that highly complementary RNA targets facilitate release of the guide RNA:target RNA duplex from AGO2.

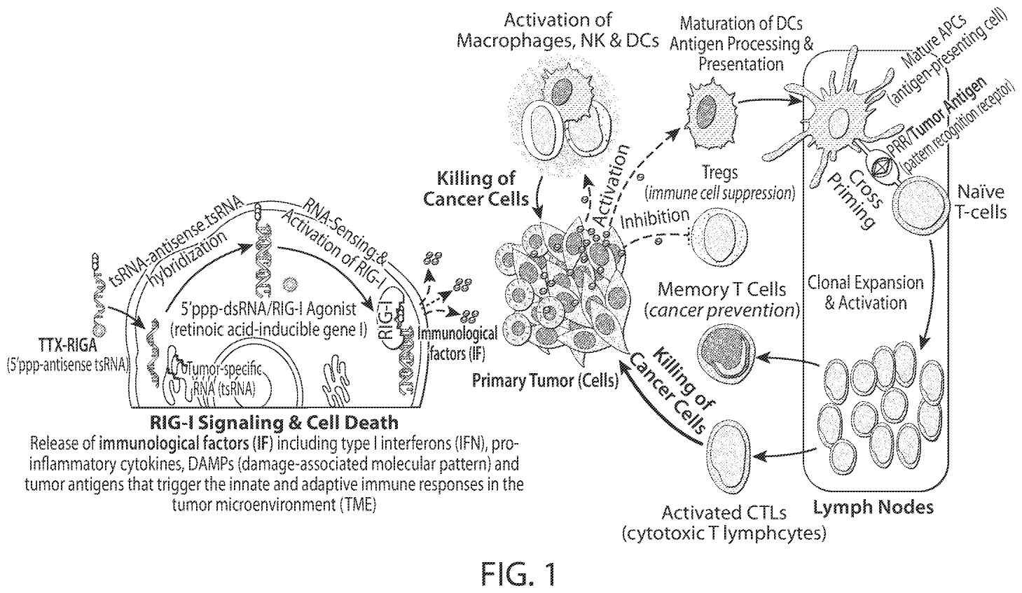

Retinoic acid-inducible gene I (RIG-I)-like receptors (RLRs) are key RNA sensors, mediating the transcriptional induction of type I interferons and other genes that collectively establish an antiviral host response (Yong H Y, Luo D. 2018; 9:1379). RIG-I is expressed in virtually all cell types, including tumor cells, and is a promising alternative to enhance ICI (immune checkpoint inhibitors) efficacy (Heidegger S. et al., 2019. EBioMedicine. 41:146. Poeck H., et al. 2008. Nat. Med. 14:1256). Preclinical studies have shown that systemic delivery of a synthetic RIG-I agonist inhibits tumor growth through mechanisms similar to those triggered for elimination of virally-infected cells (Poeck H., et al. 2008. 5′-triphosphate-siRNA: turning gene silencing and Rig-I activation against melanoma. Nat. Med. 14:1256). RIG-I engagement leads to preferential tumor cell death (via intrinsic or extrinsic apoptosis, and inflammasome-induced pyroptosis), and to IFN-1-mediated activation of the innate and adaptive immune systems (see FIG. 1 of Elion D L., et al. 2018. Oncotarget. 9:29007). RGT100, a specific RIG-I agonist, is currently in phase I/II clinical trials for treatment of advanced solid tumors and lymphomas (NCT03065023) (Elion D L., et al. 2018. Oncotarget. 9:29007).

Without being bound by theory, the RIG-I pathway may be selectively activated in cancer cells according to the methods and compositions of the present disclosure, by in situ generation of 5′ppp-dsRNA following introduction of 5′ppp RNA complementary to a miRNA (5′ppp anti-miRNA) or mRNA expressed specifically in these cells (FIG. 1). The same or similar selective activation of the RIG-I pathway is expected from 5′pp-dsRNA. Consequently, the antitumor immunity potential of the tumor microenvironment (TME) can be uncovered via the activation of RIG-I signaling pathway, in conjunction with concurrent activation of certain tumor suppressor gene(s), by simply using a single-stranded RNA.

The utility of the RIG-I agonist triphosphate RNA for melanoma therapy has been recently validated (Helms M W. et al. 2019. Utility of the RIG-I Agonist Triphosphate RNA for Melanoma Therapy. Mol Cancer Ther. 2019; 18(12):2343-2356). It is also noted that the similarity of RIG-I's natural ligand, triphosphate RNA (5′ppp-dsRNA) (and 5′pp) to small interfering RNA (siRNA) has led to the development of bifunctional siRNAs for concurrent silencing of oncogenic or immunosuppressive targets and activation of the RIG-I signaling pathway (Poeck H., et al. 2008. Nat. Med. 14:1256. Ellermeier J. et al. 2013. 2013; 73(6):1709-1720). The combined approaches mount a two-targeted attack on the tumor cells with encouraging outcomes.

MicroRNAs (miRNAs) are small non-coding RNAs that can regulate various target genes. miRNAs regulate gene expression at the post-transcriptional level through base-pairing with complementary sequences of messenger RNAs (mRNA). This interaction results in gene silencing by cleavage of the mRNA strand, destabilization of the mRNA through shortening of its polyA tail, or inhibition of translation of the mRNA into proteins. miRNAs control the expression of approximately 60% of protein-coding genes and regulate cell metabolism, proliferation, differentiation, and apoptosis (Huang Z, Shi J, Gao Y, et al. HMDD v3.0: a database for experimentally supported human microRNA-disease associations. Nucleic Acids Res. 2019; 47(D1):D1013-D1017).

Under normal physiological conditions, miRNAs function in feedback mechanisms by safeguarding key biological processes including cell proliferation, differentiation and apoptosis (Reddy, K. B., Cancer Cell International, 2015, 15:38). miRNAs are expressed in a wide variety of organs and cells, and regulate both pro- and anti-inflammatory actions. miRNAs have emerged as key regulators of the inflammatory response in a wide spectrum of human disease (Tahamtan, A., et al., Front Immunol. 2018; 9: 1377).

Dysregulation of miRNA expression has been linked to a variety of disease indications such as cancer. More than 50% of miRNA genes were revealed to be located in cancer-associated genomic regions (Di Leva, G., et al., Annu Rev Pathol. 2014; 9( ):287-314.). The dysregulation of miRNAs has been shown to perform a fundamental role in the onset, progression and dissemination of numerous types of cancer. For example, miRNA dysregulation is known to be associated with chronic lymphocytic leukemia, where miR-15a and miR-16-1 were shown to be downregulated or deleted in the majority of patients with chronic lymphocytic leukemia (Calin G. A., et al., Proc Natl Acad Sci USA; 2002; pp. 15524-15529). Other miRNAs, such as miR-21, miR-26, and miR-29a, have been shown to be preferentially expressed in cancer cells and/or the tumor cell microenvironment (Chakraborty, C., et al., Mol Ther Nucleic Acids. 2020 Jun. 5; 20: 606-620). A therapeutic methodology directed against endogenous miRNAs therefore provides a highly promising approach to target the tumor microenvironment and treat various cancers associated with dysregulated miRNAs.

RIG-I Mediated RNA-Induced Immunogenic Cell Death

The pattern recognition receptor, Retinoic acid-inducible gene I (RIG-1), recognizes specific molecular patterns of viral RNAs for inducing type I interferon. RIG-I consists of two N-terminal caspase recruitment domains (CARDs), a central RNA helicase domain, and a C-terminal RNA-binding domain. The C-terminal domain (CTD) of RIG-I recognizes the 5′-ppp group of non-self RNAs and undergoes a conformational change to induce IFN-β production (Lee, M., et al., Nucleic Acids Research, 2016, Vol. 44, No. 17). Structural and biochemical studies have demonstrated that RIG-I CTD can bind to blunt-ended dsRNAs containing a 5′-ppp. Studies have shown that 5′-ppp dsRNA strongly binds to the RIG-I CTD and stimulates interferon production more effectively compared to 5′-OH dsRNA (Pichlmair, A., et al., 2006, Science, 314, 997-1001; Vela, A., et al., 2012, J. Biol. Chem., 287, 42564-42573).

RIG-I-like receptor ligands have been used as a promising strategy for the treatment of solid malignancies including melanoma, pancreatic cancer and breast cancer in preclinical models. The major features of RIG-I are its ubiquitous expression and signaling outcomes, notably, IFN-I production and preferential tumor cell death, which are two keys factors in potent T cell responses. Despite the potential success of the RIG-I approach, the immune system is powerful and incompletely understood, warranting cautious optimism and thorough examination of the caveats associated with innate immune activation, including possible on-target induction of autoimmunity, or induction of a cytokine ‘storm’ which could pose a threat to patient safety. It is important to note that, since RIG-I is expressed in most cells of the human body, the consequences of RIG-I activation might be widespread, driving symptoms like fatigue, depression and cognitive impairment.

The present disclosure presents a strategy to mitigate the potential side effects associated with RIG-I therapy by restricting RIG-I activation to the tumor microenvironment. Specifically, tumor-specific miRNAs are used as templates for the assembly of 5′ppp-dsRNA RIG-I agonists. To accomplish this, the present methods introduce exogenously supplied 5′ppp single-stranded oligonucleotide (e.g., RNA) that is complementary to the miRNA. The complementary miRNA (endogenous) and single stranded 5′ppp oligonucleotide (e.g., RNA) (exogenous) hybridize and form a 5′ppp-dsRNA that promotes release from AGO2. The released 5′ppp-dsRNA facilitates potent activation of RIG-I signaling. Through this process, the RIG-I activation will be limited to cancer cells, essentially eliminating nonspecific immune system activation elsewhere in the body. An additional level of specificity can be achieved by coupling the exogenous single-stranded 5′ppp oligonucleotide to a nanoparticle carrier that preferentially localizes to the tumor microenvironment. As shown in FIG. 1, substitution of the 5(p)pp-anti-mRNA or-miRNA approaches described herein for standard RNAi technology for silencing target miRNA or mRNAs can promote RIG-I activation that triggers RIG-I signaling and cell death, thereby improving treatment outcomes. In vivo, 5′(p)pp-anti-mRNA/miRNA can hybridize with and silence the target mRNA or miRNA, resulting in the formation of 5′(p)pp-ds-mRNAs/-miRNAs that bind to and activate RIG-I proteins, leading to RIG-I signaling and cancer cell death.

### 2. Definitions

The terms used in this specification generally have their ordinary meanings in the art, within the context of this disclosure and in the specific context where each term is used. Certain terms are discussed below or elsewhere in the specification, to provide additional guidance to the practitioner in describing the compositions and methods of the disclosure and how to make and use them. The scope or meaning of any use of a term will be apparent from the specific context in which the term is used.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values.

Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “*about” or “approximately” can be inferred when not expressly stated.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A. B, or C; A or C; A or B; B or C; A and C; A and B; B and C; A (alone); B (alone); and C (alone).

Numeric ranges disclosed herein are inclusive of the numbers defining the ranges.

By the term “nucleic acid” is meant any single- or double-stranded polynucleotide (e.g., DNA or RNA, cDNA, semi-synthetic, or synthetic origin). The term nucleic acid includes oligonucleotides containing at least one modified nucleotide (e.g., containing a modification in the base and/or a modification in the sugar) and/or a modification in the phosphodiester bond linking two nucleotides. In some embodiments, the nucleic acid can contain at least one modified ribose such as a 2′-fluoro (2′-F). In some embodiments, the nucleic acid can contain a 5′ uncapped triphosphate or biphosphate. Non-limiting examples of nucleic acids are described herein. Additional examples of nucleic acids are known in the art.

A nucleic acid disclosed herein can comprise an oligonucleotide sequence which is not naturally occurring. Such variants necessarily have less than 100% sequence identity or similarity with the starting molecule. In certain embodiments, the variant will have a nucleic acid sequence from about 75% to less than 100% amino acid sequence identity or similarity with the nucleic acid sequence of the starting (e.g., naturally-occurring or wild-type) oligonucleotide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule. In certain aspects, the oligonucleotide sequence will be fully complementary to a target sequence. In other words, the duplex region formed by the oligonucleotide and its target will exhibit a fully complementary sequence (i.e., does not comprise any base pair mismatches or gaps) without taking into account in overhang. In certain aspects, the oligonucleotide and the target sequence does not comprise more than 0-5 base pair mismatches in the duplex region.

Tumor-specific RNAs of the present disclosure can comprise a micro RNA (miRNA) or messenger RNA (mRNA). miRNAs or mRNAs of the present disclosure may comprise oncogenic miRNAs or mRNAs. Oncogenic miRNAs or mRNAs are miRNAs or mRNAs that are believed to be involved in or associated with a tumor/tumors and/or cancer.

The term “diamagnetic” is used to describe a composition that has a relative magnetic permeability that is less than or equal to 1 and that is repelled by a magnetic field.

The term “paramagnetic” is used to describe a composition that develops a magnetic moment only in the presence of an externally applied magnetic field.

The term “ferromagnetic” is used to describe a composition that is strongly susceptible to magnetic fields and is capable of retaining magnetic properties (a magnetic moment) after an externally applied magnetic field has been removed.

By the term “nanoparticle” is meant an object that has a diameter between about 2 nm to about 200 nm (e.g., between 10 nm and 200 nm, between 2 nm and 100 nm, between 2 nm and 40 nm, between 2 nm and 30 nm, between 2 nm and 20 nm, between 2 nm and 15 nm, between 100 nm and 200 nm, and between 150 nm and 200 nm). Non-limiting examples of nanoparticles include the nanoparticles described herein.

By the term “magnetic nanoparticle” is meant a nanoparticle (e.g., any of the nanoparticles described herein) that is magnetic (as defined herein). Non-limiting examples of magnetic nanoparticles are described herein. Additional magnetic nanoparticles are known in the art.

The terms “subject” or “patient.” as used herein, refer to any mammal (e.g., a human or a veterinary subject, e.g., a dog, cat, horse, cow, goat, sheep, mouse, rat, or rabbit) to which a composition or method of the present disclosure may be administered, e.g., for experimental, diagnostic, prophylactic, and/or therapeutic purposes. The subject may seek or need treatment, require treatment, is receiving treatment, will receive treatment, or is under care by a trained professional for a particular disease or condition.

As used herein, the term “tumor” refers to an abnormal mass of tissue and/or cells in which the growth of the mass surpasses, and is not coordinated with, the growth of normal tissue, including both solid masses (e.g., as in a solid tumor) or fluid masses (e.g., as in a hematological cancer) or any cancer cell found within the tumor. A tumor can be solid (e.g., lymphoma, sarcoma or carcinoma) or non-solid (e.g., tumors of the blood, bone marrow, or lymph nodes such as leukemia). A tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue.

The term “microenvironment” as used herein means any portion or region of a tissue or body that has constant or temporal, physical or chemical differences from other regions of the tissue or regions of the body.

The term “tumor microenvironment” as used herein refers to the environment in which a tumor exists, which is the non-cellular area within the tumor and the area directly outside the tumorous tissue but does not pertain to the intracellular compartment of the cancer cell itself. It also refers cells found within the tumor microenvironment, e.g., fibroblasts, endothelial cells, adipocytes, pericytes, neuroendocrine cells, or immune cells in tumor microenvironment (macrophage, B cells, T cells etc.) The tumor and the tumor microenvironment are closely related and interact constantly. A tumor can change its microenvironment, and the microenvironment can affect how a tumor grows and spreads. Typically, the tumor microenvironment has a low pH in the range of 5.0 to 7.0, or in the range of 5.0 to 6.8, or in the range of 5.8 to 6.8, or in the range of 6.2-6.8. On the other hand, a normal physiological pH is in the range of 7.2-7.8. The tumor microenvironment is also known to have lower concentration of glucose and other nutrients, but higher concentration of lactic acid, in comparison with blood plasma. Furthermore, the tumor microenvironment can have a temperature that is 0.3 to 1° C. higher than the normal physiological temperature.

The term “non-tumor microenvironment” refers to a microenvironment at a site other than a tumor.

The term “metastasis” refers to the migration of a cancer cell present in a primary tumor to a secondary, non-adjacent tissue in a subject. Non-limiting examples of metastasis include: metastasis from a primary tumor to a lymph node (e.g., a regional lymph node), bone tissue, lung tissue, liver tissue, and/or brain tissue. The term metastasis also includes the migration of a metastatic cancer cell found in a lymph node to a secondary tissue (e.g., bone tissue, liver tissue, or brain tissue). In some non-limiting embodiments, the cancer cell present in a primary tumor is a breast cancer cell, a colon cancer cell, a kidney cancer cell, a lung cancer cell, a skin cancer cell, an ovarian cancer cell, a pancreatic cancer cell, a prostate cancer cell, a rectal cancer cell, a stomach cancer cell, a thyroid cancer cell, or a uterine cancer cell. Additional aspects and examples of metastasis are known in the art or described herein.

The term “primary tumor” refers to a tumor present at the anatomical site where tumor progression began and proceeded to yield a cancerous mass. In some embodiments, a physician may not be able to clearly identify the site of the primary tumor in a subject.

The term “metastatic tumor” refers to a tumor in a subject that originated from a tumor cell that metastasized from a primary tumor in the subject. In some embodiments, a physician may not be able to clearly identify the site of the primary tumor in a subject.

Preferred methods and materials are described herein, although methods and materials similar or equivalent to those described herein can also be used in the practice or testing of the presently disclosed methods and compositions. All publications, patent applications, patents, and other references mentioned herein are incorporated by reference in their entirety.

### 3. Endogenous Tumor-Specific RNAs

Described herein are compositions and methods for eliciting an immune response through endogenous tumor-specific RNAs. In some embodiments, the disclosure provides a method for treating cancer comprising administering a single-stranded 5′ uncapped triphosphate or biphosphate modified RNA oligonucleotide, wherein said oligonucleotide is complementary to an endogenous tumor-specific RNA which is highly expressed in tumor cells in comparison to non-tumor cells. In some embodiments, the disclosure provides a method for selectively activating RIG-I in tumor cells comprising administering a single-stranded 5′ uncapped triphosphate or biphosphate modified RNA oligonucleotide, wherein said single-stranded 5′ uncapped triphosphate or biphosphate modified RNA oligonucleotide comprises a sequence which is complementary to an endogenous tumor-specific RNA, which tumor specific RNA is specific to a tumor cell, wherein the RIG-I is selectively activated in tumor cells highly expressing the tumor-specific RNA. Endogenous tumor-specific RNAs of the present disclosure may be selected from an miRNA or mRNA. Endogenous tumor-specific RNAs of the present disclosure may be further selected from an oncogenic miRNA or oncogenic mRNA. Oncogenic miRNAs or mRNAs are miRNAs or mRNAs that are believed to be involved in cancer.

MiRNAs have been shown to be a component in many cancers and may provide novel avenues for cancer treatment. MiRNAs of the methods and compositions of the present disclosure include but are not limited to: miR-9; miR-Ob; miR-17; miR-18; miR-19b; miR-21; miR-26a; miR-29a; miR-92a; miR-106b/93; miR-125b; miR-130a; miR-155; miR-181a; miR-200s; miR-210; miR-210-3p; miR-221; miR-222; miR-221/222; miR-335; miR-498; miR-504; miR-1810; miR-1908; miR-224/452; and miR-181/340. A complete list, including sequences, is available at the OncomiRDB (Wang et al. Bioinformatics. 2014; 30(15):2237-2238; mircancer.ecu.edu/browse.jsp; US20150004221A1); see also Table 1 and Table 2).

An example of one such a miRNA is miR-10b. Upregulation of miR-10b has been shown to be responsible for migration and invasion of metastatic tumor cells as well as the viability of these cells (Tian Y., et al., J. Biol. Chem. 2010; 285:7986-7994). Analysis of miR-10b levels in 40 human esophageal cancer samples and their paired normal adjacent tissues revealed an elevated expression of miR-10b in 95% (38 of 40) of the sampled cancer tissues (Tian Y., et al., J. Biol. Chem. 2010; 285:7986-7994). There are many other miRNAs that also play a role in carcinogenesis that represent relevant targets; these and other miRNAs represent a potential new class of targets for therapeutic inhibition (Nguyen D D, Chang S. Int J Mol Sci. 2017; 19(1):65). For example, miR-21 has been shown to be involved in a variety of cancer cells and tissues, not limited to glioblastoma, breast, colorectal, lung, pancreas, skin, liver, gastric, cervical, and thyroid cancers, as well as various lymphatic, and hematopoietic cancers and neuroblastoma. miR-21 is a representative example of a single miRNA that targets multiple oncogenic signaling cascades and causes global dysregulation of gene expression networks in cancer cells (Pan, X., et al., Cancer Biol. Ther. 2010; 10:1224-1232). Increased miR-21 expression has been found to target a variety of essential tumor suppressors such as phosphatase and tensin homolog (PTEN), PDCD4, RECK, TPM1, facilitating cell proliferation, survival, metastasis, and the acquisition of a chemoresistant phenotype (Meng, F., et al., Gastroenterology. 2007; 133:647-658; Peralta-Zaragoza O., et al., BMC Cancer. 2016; 16:215; Zhang, X., et al., BMC Cancer. 2016; 16:86; Reis S T., et al., BMC Urol. 2012:12:14; Zhu S., et al., J. Biol. Chem. 2007; 282:14328-14336).

MiR-155 is epigenetically controlled by BRCA1, and is overexpressed in breast, ovarian, and lung cancers. miR-155 has been investigated as a potential biomarker for B-cell cancers. Overexpression of MiR-155 blocks B-cell differentiation via downregulation of the SHIP1 and C/EBPβ genes, and results in improved cell survival due to the activation of PI3K-Akt and MAPK pathways. In other cancers, such as glioma, overexpression of miR-155 promotes the progression of tumor formation through negative correlation with caudal-type homeobox 1 protein (CDX1) expression in glioma tissue.

MiR-210 is a well-documented miRNA implicated in various aspects of cancer development, progression and metastasis. Increased miR-210 expression was observed in bone metastatic and non-bone metastatic prostate cancer tissue. Expression was found to be elevated in bone metastatic prostate cancer tissue relative to non-bone metastatic prostate cancer tissue, and was shown to promote prostate cancer cell epithelial-mesenchymal transition and bone metastasis via the NF-κB signaling pathway (Ren D., et al., Mol Cancer. 2017; 16: 117). Other miRNAs, such as miRNA-221, have been found to be upregulated in breast cancer, glioma, hepatocellular carcinoma, pancreatic adenocarcinoma, melanoma, chronic lymphocytic leukemia, and thyroid papillary carcinoma (Brognara E., et al., Int J Oncol. 2012 December; 41(6):2119-27).

In some embodiments, the disclosure provides a method for treating cancer comprising administering a single-stranded 5′ uncapped triphosphate or biphosphate modified RNA oligonucleotide, wherein said oligonucleotide is complementary to an endogenous tumor-specific RNA which is highly expressed in tumor cells in comparison to non-tumor cells. In some embodiments, the disclosure provides a method for selectively activating RIG-I in tumor cells comprising administering a single-stranded 5′ uncapped triphosphate or biphosphate modified RNA oligonucleotide, wherein said single-stranded 5′ uncapped triphosphate or biphosphate modified RNA oligonucleotide comprises a sequence which is complementary to an endogenous tumor-specific RNA, which tumor specific RNA is specific to a tumor cell, wherein the RIG-I is selectively activated in tumor cells highly expressing the tumor-specific RNA. In some embodiments, the endogenous tumor-specific RNA is an oncogenic miRNA. In some embodiments, the endogenous tumor-specific RNA is not an oncogenic miRNA.

In some embodiments, the endogenous tumor-specific RNA is a miRNA selected from the group consisting of miR-9; miR-10b; miR-17; miR-18; miR-19b; miR-21; miR-26a; miR-29a; miR-92a; miR-106b/93; miR-125b; miR-130a; miR-155; miR-181a; miR-200s; miR-210; miR-210-3p; miR-221; miR-222; miR-221/222; miR-335; miR-498; miR-504; miR-1810; miR-1908; miR-224/452; and miR-181/340. In some embodiments, the endogenous tumor-specific RNA is miR-9. In some embodiments, the endogenous tumor-specific RNA is miR-10b. In some embodiments, the endogenous tumor-specific RNA is miR-17. In some embodiments, the endogenous tumor-specific RNA is miR-18. In some embodiments, the endogenous tumor-specific RNA is miR-19b. In some embodiments, the endogenous tumor-specific RNA is miR-21. In some embodiments, the endogenous tumor-specific RNA is miR-26a. In some embodiments, the endogenous tumor-specific RNA is miR-29a. In some embodiments, the endogenous tumor-specific RNA is miR-92a. In some embodiments, the endogenous tumor-specific RNA is miR-106b/93. In some embodiments, the endogenous tumor-specific RNA is miR-125b. In some embodiments, the endogenous tumor-specific RNA is miR-130a. In some embodiments, the endogenous tumor-specific RNA is miR-155. In some embodiments, the endogenous tumor-specific RNA is miR-181a. In some embodiments, the endogenous tumor-specific RNA is miR-200s. In some embodiments, the endogenous tumor-specific RNA is miR-210. In some embodiments, the endogenous tumor-specific RNA is miR-210-3p. In some embodiments, the endogenous tumor-specific RNA is miR-221. In some embodiments, the endogenous tumor-specific RNA is miR-222. In some embodiments, the endogenous tumor-specific RNA is miR-221/222. In some embodiments, the endogenous tumor-specific RNA is miR-335. In some embodiments, the endogenous tumor-specific RNA is miR-498. In some embodiments, the endogenous tumor-specific RNA is miR-504. In some embodiments, the endogenous tumor-specific RNA is miR-1810. In some embodiments, the endogenous tumor-specific RNA is miR-1908. In some embodiments, the endogenous tumor-specific RNA is miR-224/452. In some embodiments, the endogenous tumor-specific RNA is miR-181/340.

In a preferred embodiment of the present disclosure, the endogenous tumor-specific RNA is selected from the group consisting of: miR10b, miR17, miR18a, miR18b, miR19b, miR21. miR26a, miR29a, miR92a-1, miR92a-2, miR155, miR210, and miR221. In some embodiments, the endogenous tumor-specific RNA is miR10b. In some embodiments, the endogenous tumor-specific RNA is miR17. In some embodiments, the endogenous tumor-specific RNA is miR18a. In some embodiments, the endogenous tumor-specific RNA is miR18b. In some embodiments, the endogenous tumor-specific RNA is miR19b. In some embodiments, the endogenous tumor-specific RNA is miR21. In some embodiments, the endogenous tumor-specific RNA is miR26a. In some embodiments, the endogenous tumor-specific RNA is miR29a. In some embodiments, the endogenous tumor-specific RNA is miR92a-1. In some embodiments, the endogenous tumor-specific RNA is miR92a-2. In some embodiments, the endogenous tumor-specific RNA is miR155. In some embodiments, the endogenous tumor-specific RNA is miR210. In some embodiments, the endogenous tumor-specific RNA is miR22.

In some embodiments, the endogenous tumor-specific RNA which is highly expressed in tumor cells is selected from the group consisting of: miR-9; miR-10b; miR-17; miR-18; miR-19b; miR-21; miR-26a; miR-29a; miR-92a; miR-106b/93; miR-125b; miR-130a; miR-155; miR-181a; miR-200s; miR-210; miR-210-3p; miR-221; miR-222; miR-221/222; miR-335; miR-498; miR-504; miR-1810; miR-1908; miR-224/452; and miR-181/340. In some embodiments, the tumor cell is associated with bone and non-bone metastatic cancers, breast cancer, glioma, hepatocellular carcinoma, pancreatic adenocarcinoma, melanoma, chronic lymphocytic leukemia, thyroid papillary carcinoma, glioblastoma, colorectal cancer, lung cancer, kidney cancer, pancreatic cancer, skin cancer, liver cancer, gastric cancer, cervical cancer, thyroid cancers, lymphatic cancers, hematopoietic cancers, neuroblastoma, acute myeloid leukemia, esophageal cancer, osteosarcoma, B-cell lymphoma, lymphoid leukemia, ovarian cancer, oral cancer, bladder cancer, adenoid cystic carcinoma, anaplastic thyroid carcinoma, astrocytoma, meningioma, retinoblastoma.

In some embodiments, the tumor cell is associated with bone metastatic cancer. In some embodiments, the tumor cell is associated with non-bone metastatic cancer. In some embodiments, the tumor cell is associated with breast cancer. In some embodiments, the tumor cell is associated with glioma. In some embodiments, the tumor cell is associated with hepatocellular carcinoma. In some embodiments, the tumor cell is associated with pancreatic adenocarcinoma. In some embodiments, the tumor cell is associated with melanoma. In some embodiments, the tumor cell is associated with chronic lymphocytic leukemia. In some embodiments, the tumor cell is associated with thyroid papillary carcinoma. In some embodiments, the tumor cell is associated with glioblastoma. In some embodiments, the tumor cell is associated with colorectal cancer. In some embodiments, the tumor cell is associated with lung cancer. In some embodiments, the tumor cell is associated with kidney cancer. In some embodiments, the tumor cell is associated with pancreatic cancer. In some embodiments, the tumor cell is associated with skin cancer. In some embodiments, the tumor cell is associated with liver cancer. In some embodiments, the tumor cell is associated with gastric cancer. In some embodiments, the tumor cell is associated with cervical cancer. In some embodiments, the tumor cell is associated with thyroid cancer. In some embodiments, the tumor cell is associated with lymphatic cancers. In some embodiments, the tumor cell is associated with hematopoictic cancers. In some embodiments, the tumor cell is associated with neuroblastoma. In some embodiments, the tumor cell is associated with acute myeloid leukemia. In some embodiments, the tumor cell is associated with esophageal cancer. In some embodiments, the tumor cell is associated with osteosarcoma. In some embodiments, the tumor cell is associated with B-cell lymphoma. In some embodiments, the tumor cell is associated with lymphoid leukemia. In some embodiments, the tumor cell is associated with ovarian cancer. In some embodiments, the tumor cell is associated with oral cancer. In some embodiments, the tumor cell is associated with bladder cancer. In some embodiments, the tumor cell is associated with adenoid cystic carcinoma. In some embodiments, the tumor cell is associated with anaplastic thyroid carcinoma. In some embodiments, the tumor cell is associated with astrocytoma. In some embodiments, the tumor cell is associated with meningioma. In some embodiments, the tumor cell is associated with retinoblastoma.

Many web-based tools for identifying microRNAs involved in human cancer are available. For a review, see Mar-Aguilar F, Rodriguez-Padilla C, Reséndez-Pérez D. Web-based tools for microRNAs involved in human cancer, Oncol Lett. 2016; 11(6):3563-3570. The databases can be mined for miRNAs associated with a particular type of cancer, or the behavior of a particular miRNA in different malignancies at the same time, and the sequences of a specific miRNA can be readily retrieved from various databases. For example, miRCancer (mircancer.ecu.edu) is a database that stores records of miRNA and cancer associations collected through data mining A rule-based approach was devised to analyze the title and abstract of 26,414 publications (2016) and to find full sentences or phrases that included the names of the miRNA and the cancer type, and any expression terms. The results of this data mining process were then corroborated by hand. miRCancer has records of >3,764 miRNA-cancer associations from 2,611 publications, which amounts to 236 miRNA expression profiles from 176 human cancers. miRCancer is freely accessible online, and the database can be searched by miRNA name or cancer type, or a combination of both (Xie B, Ding Q, Han H, Wu D. miRCancer: a microRNA-cancer association database constructed by text mining on literature. Bioinformatics. 2013; 29(5):638-644).

As an example, mining of the database (Dec. 16, 2020) found miR-10b to be upregulated in 20 types of cancers including acute myeloid leukemia, bladder cancer, colorectal cancer, endometrial cancer, esophageal cancer, esophageal squamous cell carcinoma, gastric cancer, gastric cancer, glioblastoma, glioma, hepatocellular carcinoma, lung cancer, malignant melanoma, medulloblastoma, nasopharyngeal carcinoma, non-small cell lung cancer, oral cancer, osteosarcoma, pancreatic cancer, and pancreatic ductal adenocarcinoma. Similarly, over 100 miRNAs including miR-10b were found to be related to breast cancer including hsa-miR-101, hsa-miR-106a, hsa-miR-106b, hsa-miR-10b, hsa-miR-1207-5p, hsa-miR-1228, hsa-miR-1229, hsa-miR-1246, hsa-miR-125a, hsa-miR-125b, hsa-miR-1307-3p, hsa-miR-135a, hsa-miR-140, hsa-miR-141, hsa-miR-150, hsa-miR-150-5p, hsa-miR-153, hsa-miR-155, hsa-miR-17, hsa-miR-17-5p, hsa-miR-181a, hsa-miR-181b, hsa-miR-181b-3p, hsa-miR-182, hsa-miR-182-5p, hsa-miR-183, hsa-miR-183-5p, hsa-miR-18a, hsa-miR-18b, hsa-miR-191, hsa-miR-1915-3p, hsa-miR-196a, hsa-miR-197, hsa-miR-19a, hsa-miR-19b, hsa-miR-200a, hsa-miR-200a-3p, hsa-miR-200b, hsa-miR-200c, hsa-miR-203, hsa-miR-205, hsa-miR-205-5p, hsa-miR-206, hsa-miR-20a, hsa-miR-20b, hsa-miR-21, hsa-miR-214-3p, hsa-miR-217, hsa-miR-221, hsa-miR-222, hsa-miR-223, hsa-miR-224, hsa-miR-224-5p, hsa-miR-23a, hsa-miR-23b, hsa-miR-24, hsa-miR-24-2-5p, hsa-miR-24-3p, hsa-miR-27a, hsa-miR-27b, hsa-miR-29a, hsa-miR-301a-3p, hsa-miR-3136-3p, hsa-miR-3188, hsa-miR-32, hsa-miR-330-3p, hsa-miR-346, hsa-miR-3646, hsa-miR-370, hsa-miR-372, hsa-miR-372-3p, hsa-miR-373, hsa-miR-374a, hsa-miR-376b, hsa-miR-378, hsa-miR-423, hsa-miR-429, hsa-miR-4469, hsa-miR-449a, hsa-miR-4513, hsa-miR-4530, hsa-miR-4732-5p, hsa-miR-494, hsa-miR-495, hsa-miR-498, hsa-miR-5003-3p, hsa-miR-503, hsa-miR-503-3p, hsa-miR-510, hsa-miR-520c, hsa-miR-520e, hsa-miR-520g, hsa-miR-526b, hsa-miR-544a, hsa-miR-645, hsa-miR-655, hsa-miR-660-5p, hsa-miR-665, hsa-miR-675, hsa-miR-761, hsa-miR-762, hsa-miR-9, hsa-miR-92a, hsa-miR-92a-3p, hsa-miR-93, hsa-miR-93-5p, hsa-miR-937, hsa-miR-944, hsa-miR-96, and hsa-miR-96-5p.

The sequence of miR-10b or any sequence of interest can be retrieved from miRbase, the microRNA database (mirbase.org/):

>hsa-miR-10b-5p MIMAT0000254
UACCCUGUAGAACCGAAUUUGUG
>hsa-miR-10b-3p MIMAT0004556
ACAGAUUCGAUUCUAGGGGAAU.

TABLE 1
Sequences of upregulated miRNAs associated with certain cancers

| | | | Cancer |
|---|---|---|---|
| microRNA ID | Accession # | Sequence (5′-3′) | (partial list) |
| hsa-miR-9-5p | MIMAT0000441 | UCUUUGGUUAUCUAGCUGUA | Breast, cervical, |
| | | UGA (SEQ ID NO: 27) | and glioma |
| | | | cancer |
| hsa-let-7a-5p | MIMAT0000062 | UGAGGUAGUAGGUUGUAUAG | Acute myeloid |
| | | UU (SEQ ID NO: 28) | leukemia |
| hsa-miR- | MIMAT0000680 | UAAAGUGCUGACAGUGCAGA | Breast cancer, |
| 106b-5p | | U (SEQ ID NO: 29) | cervical, |
| | | | colorectal and |
| | | | gastric cancer |
| hsa-miR- | MIMAT0000254 | UACCCUGUAGAACCGAAUUU | Glioblastoma, |
| 10b-5p | | GUG (SEQ ID NO: 14) | esophageal and |
| | | | breast cancer |
| hsa-miR- | MIMAT0004593 | GCUCUUUUCACAUUGUGCUA | Gastric cancer, |
| 130a-5p | | CU (SEQ ID NO: 30) | cervical and |
| | | | osteosarcoma |
| | | | cancer |
| hsa-miR- | MIMAT0000449 | UGAGAACUGAAUUCCAUGGG | Hepatocellular |
| 146a-5p | | UU (SEQ ID NO: 31) | carcinoma, |
| | | | cervical and |
| | | | colorectal |
| | | | cancer |
| hsa-miR- | MIMAT0000646 | UUAAUGCUAAUCGUGAUAGG | Liver, lung, |
| 155-5p | | GGUU (SEQ ID NO: 24) | kidney, glioma |
| | | | and pancreatic |
| | | | cancer; B cell |
| | | | lymphoma and |
| | | | lymphoid |
| | | | leukemia |
| hsa-miR- | MIMAT0000256 | AACAUUCAACGCUGUCGGUG | Cervical, |
| 18la-5p | | AGU (SEQ ID NL: 32) | Breast, cervical, |
| | | | colon, and |
| | | | gastric cancer |
| hsa-miR- | MIMAT0000257 | AACAUUCAUUGCUGUCGGUG | Breast cancer, |
| 181b-5p | | GGU (SEQ ID NO: 33) | cervical, |
| | | | osteosarcoma, |
| | | | ovarian and |
| | | | prostate cancer |
| hsa-miR- | MIMAT0007881 | CGGCGGGGACGGCGAUUGGU | Glioblastoma, |
| 1908-5p | | C (SEQ ID NO: 34) | osteosarcoma |
| hsa-miR- | MIMAT0001620 | CAUCUUACCGGACAGUGCUG | Breast, ovarian |
| 200a-5p | | GA (SEQ ID NO: 35) | and esophageal |
| | | | cancer |
| hsa-miR- | MIMAT0000267 | CUGUGCGUGUGACAGCGGCU | Prostate cancer |
| 210-3p | | GA (SEQ ID NO: 36) | |
| hsa-miR- | MIMAT0026475 | AGCCCCUGCCCACCGCACACU | Cervical, |
| 210-5p | | G (SEQ ID NO: 25) | colorectal, |
| | | | esophageal, |
| | | | glioma and lung |
| | | | cancer |
| hsa-miR- | MIMAT0004564 | UGCCUGUCUACACUUGCUGU | Oral, gastric and |
| 214-5p | | GC (SEQ ID NO: 37) | pancreatic |
| | | | cancer |
| hsa-miR-21-5p | MIMAT0000076 | UAGCUUAUCAGACUGAUGUU | Glioblastoma, |
| | | GA (SEQ ID NO: 19) | breast, |
| | | | colorectal, lung, |
| | | | pancreas, liver, |
| | | | gastric, cervical |
| | | | and |
| | | | hematopoietic |
| | | | cancer |
| hsa-miR- | MIMAT0004568 | ACCUGGCAUACAAUGUAGAU | Bladder, breast, |
| 221-5p | | UU (SEQ ID NO: 26) | cervical, colon, |
| | | | gastric and liver |
| | | | cancer |
| hsa-miR- | MIMAT0004569 | CUCAGUAGCCAGUGUAGAUC | Adenoid cystic |
| 222-5p | | CU (SEQ ID NO: 38) | carcinoma, |
| | | | anaplastic |
| | | | thyroid |
| | | | carcinoma, |
| | | | bladder and |
| | | | breast cancer |
| hsa-miR-224-5p | MIMAT0000281 | UCAAGUCACUAGUGGUUCCG | Bladder, breast, |
| | | UUUAG (SEQ ID NO: 39) | cervical, |
| | | | colorectal |
| | | | cancer and |
| | | | Glioblastoma |
| hsa-miR-335-5p | MIMAT0000765 | UCAAGAGCAAUAACGAAAAA | astrocytoma, |
| | | UGU (SEQ ID NO: 40) | colorectal cancer |
| | | | and |
| | | | meningioma |
| hsa-miR- | MIMAT0004692 | UUAUAAAGCAAUGAGACUGA | Gastric and |
| 340-5p | | UU (SEQ ID NO: 41) | thyroid cancer |
| hsa-miR-452-5p | MIMAT0001635 | AACUGUUUGCAGAGGAAACU | Hepatocellular |
| | | GA (SEQ ID NO: 42) | carcinoma, |
| | | | colorectal and |
| | | | esophageal |
| | | | cancer |
| hsa-miR-498-5p | MIMAT0002824 | UUUCAAGCCAGGGGGCGUUU | Breast cancer and |
| | | UUC (SEQ ID NO: 43) | retinoblastoma |
| hsa-miR- | MIMAT0002875 | AGACCCUGGUCUGCACUCUA | Osteosarcoma |
| 504-5p | | UC (SEQ ID NO: 44) | |
| hsa-miR-93-5p | MIMAT0000093 | CAAAGUGCUGUUCGUGCAGG | Breast, cervical |
| | | UAG (SEQ ID NO: 45) | and lung cancer |

TABLE 2
Select Sequences/reverse complement sequences for miRNAs

| miRNA | | |
|---|---|---|
| (human) | miRNA 5P Sequence | Reverse Complement |
| miR10b | uacccuguagaaccgaauuugug (SEQ ID NO: 14) | cacaaauucgguucuacagggua (SEQ ID NO: 1) |
| miR17 | caaagugcuuacagugcagguag (SEQ ID NO: 15) | cuaccugcacuguaagcacuuug (SEQ ID NO: 2) |
| miR18a | uaaggugcaucuagugcagauag (SEQ ID NO: 16) | cuaucugcacuagaugcaccuua (SEQ ID NO: 3) |
| miR18b | uaaggugcaucuagugcaguuag (SEQ ID NO: 17) | cuaacugcacuagaugcaccuua (SEQ ID NO: 4) |
| miR19b | aguuuugcagguuugcauccagc (SEQ ID NO: 18) | gcuggaugcaaaccugcaaaacu (SEQ ID NO: 5) |
| miR21 | uagcuuaucagacugauguuga (SEQ ID NO: 19) | ucaacaucagucugauaagcua (SEQ ID NO: 6) |
| miR26a | uucaaguaaauccaggauaggcu (SEQ ID NO: 20) | agccuauccuggauuuacuugaa (SEQ ID NO: 7) |
| miR29a | acugauuucuuuugguguucag (SEQ ID NO: 21) | cugaacaccaaaagaaaucagu (SEQ ID NO: 8) |
| miR92a-1 | agguugggaucgguugcaaugcu (SEQ ID NO: 22) | agcauugcaaccgaucccaaccu (SEQ ID NO: 9) |
| miR92a-2 | ggguggggauuuguugcauuac (SEQ ID NO: 23) | guaaugcaacaaauccccaccc (SEQ ID NO: 10) |
| miR155 | uuaaugcuaaucgugauagggguu (SEQ ID NO: 24) | aaccccuaucacgauuagcauuaa (SEQ ID NO: 11) |
| miR210 | agccccugcccaccgcacacug (SEQ ID NO: 25) | cagugugcggugggcaggggcu (SEQ ID NO: 12) |
| miR221 | accuggcauacaauguagauuu (SEQ ID NO: 26) | aaaucuacauuguaugccaggu (SEQ ID NO: 13) |

The methods and compositions of the present disclosure may be extended to other RNA targets such as mRNAs coding for a protein that promotes cancer development. In some embodiments, the disclosure provides a method for treating cancer comprising administering a single-stranded 5′ uncapped triphosphate or biphosphate modified RNA oligonucleotide, wherein said oligonucleotide is complementary to an endogenous tumor-specific RNA which is highly expressed in tumor cells in comparison to non-tumor cells. In some embodiments, the disclosure provides a method for selectively activating RIG-I in tumor cells comprising administering a single-stranded 5′ uncapped triphosphate or biphosphate modified RNA oligonucleotide, wherein said single-stranded 5′ uncapped triphosphate or biphosphate modified RNA oligonucleotide comprises a sequence which is complementary to an endogenous tumor-specific RNA, which tumor specific RNA is specific to a tumor cell, wherein the RIG-I is selectively activated in tumor cells highly expressing the tumor-specific RNA. In some embodiments, the endogenous tumor-specific RNA is an mRNA. In some embodiments, the endogenous tumor-specific RNA is an mRNA. In some embodiments, the endogenous tumor-specific RNA is not an mRNA.

A number of mRNAs are believed to be involved in cancer. The entire or partial antisense strand of the mRNA including the poly-A tail can be generated from a DNA template by in vitro transcription, and modified with 5′(p)pp. The 5′(p)pp-anti-mRNA sequence can be optimized to contain sequence elements that increase RNA stability. An anti-mRNA may be formulated with lipids to obtain an RNA-lipid nanoparticle drug product. In vivo, 5′(p)pp-anti-mRNA can hybridize with and silence the target mRNA resulting in the formation of 5′(p)pp-ds-mRNAs that bind and activate RIG-I proteins, and lead to RIG-I signaling and cancer cell death. See Table 3 for a list of exemplary mRNA transcripts.

TABLE 3
Select oncogenes and their RefSeq accession numbers

| Oncogene Category | Examples | mRNA Accession # |
|---|---|---|
| Cytoplasmic tyrosine | Src- | NM_005417.5, NM_198291.3 |
| kinases | family | |
| Cytoplasmic Serine/ | RAF | NM_002880.4 |
| threonine kinases and | kinase | |
| their regulatory | | |
| subunits | | |
| Regulatory GTPases | RAS | NM_001130442.3, NM_005343.4, |
| | protein | NM_176795.5, NM_001318054.2 |
| | (HRas) | |
| | RAS | NM_001130442.3, NM_033360.4, |
| | protein | NM_001369786.1, |
| | (KRas) | NM_001369787.1 |
| | RAS | NM_002524.5 |
| | protein | |
| | (NRas) | |
| Transcription factors | MYC | NM_005376.5, NM_001354870.1, |
| | gene | NM_005378.6 |
| Inhibitor of apoptosis | BIRC5 | NM_001012270.2, |
| (IAP) family | | NM_001012271.2, NM_001168.3 |

For example, Survivin (also named BIRC5), a well-known cancer therapeutic target, can be targeted using this approach. Survivin, a multi-regulator of cell cycle and apoptosis is overexpressed in all human cancers but demonstrates low expression in normal tissues. Its increased expression has been detected in 90% of primary breast cancers and correlates with poor clinical outcomes. Furthermore, increased surviving levels have been shown to be significantly associated with negative hormone receptor status. Importantly, high levels of survivin have been detected in other cancers such as pancreatic cancer, where it correlates with both cellular proliferation and apoptosis pointing to a possible ubiquitous role of this anti-apoptotic marker. Considering the potential value of reducing or abolishing survivin expression as a means of overcoming chemoresistance, the process of RNA interference (RNAi) can prove valuable. Indeed, down-regulation of BIRC5 by RNAi demonstrated promise in acute lymphoblastic leukemia, lung, and cervical carcinoma in vitro and breast cancer in vivo (Ghosh S K, Yigit M V, Uchida M, et al. Sequence-dependent combination therapy with doxorubicin and a survivin-specific small interfering RNA nanodrug demonstrates efficacy in models of adenocarcinoma. Int J Cancer. 2014; 134(7):1758-1766). Sequence-dependent combination therapy with doxorubicin and a survivin-specific small interfering RNA nanodrug demonstrates efficacy in models of adenocarcinoma.

Substitution of the current 5(p)pp-anti-mRNA approach for standard siRNA technology for silencing survivin can promote RIG-I activation that triggers RIG-I signaling and cell death, thereby improving treatment outcome.

### 4. Oligonucleotides and Oligonucleotide Modifications

In certain embodiments, the disclosure provides methods and compositions comprising single-stranded 5′ uncapped triphosphate or biphosphate modified RNA oligonucleotides, wherein said oligonucleotide is complementary to an endogenous tumor-specific RNA which is highly expressed in tumor cells in comparison to non-tumor cells. In certain embodiments, the disclosure provides methods and compositions comprising a single-stranded 5′ uncapped triphosphate or biphosphate modified RNA oligonucleotide, wherein said single-stranded 5′ uncapped triphosphate or biphosphate modified RNA oligonucleotide comprises a sequence which is complementary to an endogenous tumor-specific RNA.

Exogenous RNA comprising a 5′ triphosphate (ppp) has been shown to induce an immunogenic form of cell death in different tumor entities (Elion, D L., et al Cancer Res. 2018 Nov. 1; 78(21):6183-6195; Besch, R., et al, J Clin Investig. 2009; 119:2399-411; Duewell, P., et al., Cell Death Differ. 2014:21:1825-37; Kuber, K., et al., Cancer Res. 2010; 70:5293-304). 5′ biphosphate (5′pp) or 5′ triphosphate modification (5′ppp), may be referred to herein as 5′pp and 5′ppp anti-miRNAs/mRNAs, respectively. 5′-ppp-RNA has been shown to induce cytokine release combined with direct sensing of viral RNA by immune cells, and promote an adaptive cellular immune response directed against tumor cells (Poeck, H., et al., Nat Med. 2008 November; 14(11):1256-63). The pattern recognition receptor, RIG-I, can bind to blunt-ended dsRNAs containing an uncapped 5′ppp or 5′pp. As disclosed herein, uncapped refers to an RNA lacking a 5′ cap structure consisting of a 7-methylguansine triphosphate linked to the 5′ end of the mRNA via a 5′-5′ triphosphate linkage. The present disclosure provides a method for selectively activating RIG-I in tumor cells comprising administering a single-stranded 5′ uncapped triphosphate or biphosphate modified RNA oligonucleotide, wherein said single-stranded 5′ uncapped triphosphate or biphosphate modified RNA oligonucleotide comprises a sequence which is complementary to an endogenous tumor-specific RNA. The present disclosure also provides a single-stranded 5′ uncapped triphosphate or biphosphate modified RNA oligonucleotide, wherein said oligonucleotide is complementary to a miRNA, which is highly expressed in tumor tissue in comparison to non-tumor tissue. The 5′triphosphate structure is shown below:

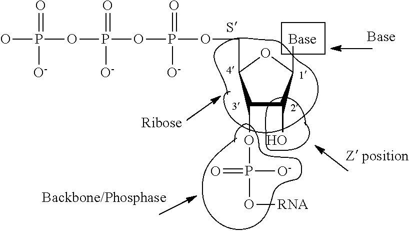

In a preferred embodiment, the single-stranded 5′ uncapped triphosphate or biphosphate modified RNA oligonucleotide comprises an uncapped 5′ triphosphate. In a preferred embodiment, the single-stranded 5′ uncapped triphosphate or biphosphate modified RNA oligonucleotide comprises an uncapped 5′ biphosphate.

In some embodiments of the methods and compositions disclosed herein, the single-stranded 5′ uncapped triphosphate or biphosphate modified RNA oligonucleotide comprises a sequence which is complementary to an miRNA. In some embodiments, the single-stranded 5′ uncapped triphosphate or biphosphate modified RNA oligonucleotide comprises a sequence which is complementary to an endogenous miRNA. In some embodiments, the single-stranded 5′ uncapped triphosphate or biphosphate modified RNA oligonucleotide comprises a sequence which is complementary to an oncogenic miRNA selected from the group consisting of: miR-9; miR-10b; miR-17; miR-18; miR-19b; miR-21; miR-26a; miR-29a; miR-92a; miR-106b/93; miR-125b; miR-130a; miR-155; miR-181a; miR-200s; miR-210; miR-210-3p; miR-221; miR-222; miR-221/222; miR-335; miR-498; miR-504; miR-1810; miR-1908; miR-224/452; and miR-181/340. In some embodiments, the single-stranded 5′ uncapped triphosphate or biphosphate modified RNA oligonucleotide comprises a sequence which is complementary to miR-9. In some embodiments, the single-stranded 5′ uncapped triphosphate or biphosphate modified RNA oligonucleotide comprises a sequence which is complementary to miR-10b. In some embodiments, the single-stranded 5′ uncapped triphosphate or biphosphate modified RNA oligonucleotide comprises a sequence which is complementary to miR-17. In some embodiments, the single-stranded 5′ uncapped triphosphate or biphosphate modified RNA oligonucleotide comprises a sequence which is complementary to miR-18. In some embodiments, the single-stranded 5′ uncapped triphosphate or biphosphate modified RNA oligonucleotide comprises a sequence which is complementary to miR-19b. In some embodiments, the single-stranded 5′ uncapped triphosphate or biphosphate modified RNA oligonucleotide comprises a sequence which is complementary to miR-21. In some embodiments, the single-stranded 5′ uncapped triphosphate or biphosphate modified RNA oligonucleotide comprises a sequence which is complementary to miR-26a. In some embodiments, the single-stranded 5′ uncapped triphosphate or biphosphate modified RNA oligonucleotide comprises a sequence which is complementary to miR-29a. In some embodiments, the single-stranded 5′ uncapped triphosphate or biphosphate modified RNA oligonucleotide comprises a sequence which is complementary to miR-92a. In some embodiments, the single-stranded 5′ uncapped triphosphate or biphosphate modified RNA oligonucleotide comprises a sequence which is complementary to miR-106b/93. In some embodiments, the single-stranded 5′ uncapped triphosphate or biphosphate modified RNA oligonucleotide comprises a sequence which is complementary to miR-125b. In some embodiments, the single-stranded 5′ uncapped triphosphate or biphosphate modified RNA oligonucleotide comprises a sequence which is complementary to miR-130a. In some embodiments, the single-stranded 5′ uncapped triphosphate or biphosphate modified RNA oligonucleotide comprises a sequence which is complementary to miR-155. In some embodiments, the single-stranded 5′ uncapped triphosphate or biphosphate modified RNA oligonucleotide comprises a sequence which is complementary to miR-181a. In some embodiments, the single-stranded 5′ uncapped triphosphate or biphosphate modified RNA oligonucleotide comprises a sequence which is complementary to miR-200s. In some embodiments, the single-stranded 5′ uncapped triphosphate or biphosphate modified RNA oligonucleotide comprises a sequence which is complementary to miR-210. In some embodiments, the single-stranded 5′ uncapped triphosphate or biphosphate modified RNA oligonucleotide comprises a sequence which is complementary to miR-210-3p. In some embodiments, the single-stranded 5′ uncapped triphosphate or biphosphate modified RNA oligonucleotide comprises a sequence which is complementary to miR-221. In some embodiments, the single-stranded 5′ uncapped triphosphate or biphosphate modified RNA oligonucleotide comprises a sequence which is complementary to miR-222. In some embodiments, the single-stranded 5′ uncapped triphosphate or biphosphate modified RNA oligonucleotide comprises a sequence which is complementary to miR-221/222. In some embodiments, the single-stranded 5′ uncapped triphosphate or biphosphate modified RNA oligonucleotide comprises a sequence which is complementary to miR-335. In some embodiments, the single-stranded 5′ uncapped triphosphate or biphosphate modified RNA oligonucleotide comprises a sequence which is complementary to miR-498. In some embodiments, the single-stranded 5′ uncapped triphosphate or biphosphate modified RNA oligonucleotide comprises a sequence which is complementary to miR-504. In some embodiments, the single-stranded 5′ uncapped triphosphate or biphosphate modified RNA oligonucleotide comprises a sequence which is complementary to miR-1810. In some embodiments, the single-stranded 5′ uncapped triphosphate or biphosphate modified RNA oligonucleotide comprises a sequence which is complementary to miR-1908. In some embodiments, the single-stranded 5′ uncapped triphosphate or biphosphate modified RNA oligonucleotide comprises a sequence which is complementary to miR-224/452. In some embodiments, the single-stranded 5′ uncapped triphosphate or biphosphate modified RNA oligonucleotide comprises a sequence which is complementary to miR-181/340.

In a preferred embodiment of the present disclosure, the single-stranded 5′ uncapped triphosphate or biphosphate modified RNA oligonucleotide comprises a sequence which is complementary to miR10b, miR17, miR18a, miR18b, miR19b, miR21, miR26a, miR29a, miR92a-1, miR92a-2, miR155, miR210, and miR221. In some embodiments, the single-stranded 5′ uncapped triphosphate or biphosphate modified RNA oligonucleotide comprises a sequence which is complementary to miR10b. In some embodiments, the single-stranded 5′ uncapped triphosphate or biphosphate modified RNA oligonucleotide comprises a sequence which is complementary to miR17. In some embodiments, the single-stranded 5′ uncapped triphosphate or biphosphate modified RNA oligonucleotide comprises a sequence which is complementary to miR18a. In some embodiments, the single-stranded 5′ uncapped triphosphate or biphosphate modified RNA oligonucleotide comprises a sequence which is complementary to miR18b. In some embodiments, the single-stranded 5′ uncapped triphosphate or biphosphate modified RNA oligonucleotide comprises a sequence which is complementary to miR19b. In some embodiments, the single-stranded 5′ uncapped triphosphate or biphosphate modified RNA oligonucleotide comprises a sequence which is complementary to miR21. In some embodiments, the single-stranded 5′ uncapped triphosphate or biphosphate modified RNA oligonucleotide comprises a sequence which is complementary to miR26a. In some embodiments, the single-stranded 5′ uncapped triphosphate or biphosphate modified RNA oligonucleotide comprises a sequence which is complementary to miR29a. In some embodiments, the single-stranded 5′ uncapped triphosphate or biphosphate modified RNA oligonucleotide comprises a sequence which is complementary to miR92a-1. In some embodiments, the single-stranded 5′ uncapped triphosphate or biphosphate modified RNA oligonucleotide comprises a sequence which is complementary to miR92a-2. In some embodiments, the single-stranded 5′ uncapped triphosphate or biphosphate modified RNA oligonucleotide comprises a sequence which is complementary to miR155. In some embodiments, the single-stranded 5′ uncapped triphosphate or biphosphate modified RNA oligonucleotide comprises a sequence which is complementary to miR210. In some embodiments, the single-stranded 5′ uncapped triphosphate or biphosphate modified RNA oligonucleotide comprises a sequence which is complementary to miR22.

In some embodiments of the methods and compositions disclosed herein, the single-stranded 5′ uncapped triphosphate or biphosphate modified RNA oligonucleotide forms a duplex with the miRNA. In a preferred embodiment, the duplex comprises a 5′ blunt end. In some embodiments, the single-stranded 5′ uncapped triphosphate or biphosphate modified RNA oligonucleotide forms a duplex with a miRNA selected from the group consisting of: miR-9; miR-10b; miR-17; miR-18; miR-19b; miR-21; miR-26a; miR-29a; miR-92a; miR-106b/93; miR-125b; miR-130a; miR-155; miR-181a; miR-200s; miR-210; miR-210-3p; miR-221; miR-222; miR-221/222; miR-335; miR-498; miR-504; miR-1810; miR-1908; miR-224/452; and miR-181/340. In a preferred embodiment, the single-stranded 5′ uncapped triphosphate or biphosphate modified RNA oligonucleotide forms a duplex with a miRNA selected from the group consisting of: miR10b, miR17, miR18a, miR18b, miR19b, miR21, miR26a, miR29a, miR92a-1, miR92a-2, miR155, miR210, and miR221. In some embodiments, the single-stranded 5′ uncapped triphosphate or biphosphate modified RNA oligonucleotide is capable of forming a duplex with said miRNA, wherein the duplexed portion of the oligonucleotide is complementary to at least 10 contiguous nucleotides within a miRNA. In some embodiments, the duplexed portion of the oligonucleotide is complementary to at least 11 contiguous nucleotides within a miRNA. In some embodiments, the duplexed portion of the oligonucleotide is complementary to at least 12 contiguous nucleotides within a miRNA. In some embodiments, the duplexed portion of the oligonucleotide is complementary to at least 13 contiguous nucleotides within a miRNA. In some embodiments, the duplexed portion of the oligonucleotide is complementary to at least 14 contiguous nucleotides within a miRNA. In some embodiments, the duplexed portion of the oligonucleotide is complementary to at least 15 contiguous nucleotides within a miRNA. In some embodiments, the duplexed portion of the oligonucleotide is complementary to at least 16 contiguous nucleotides within a miRNA. In some embodiments, the duplexed portion of the oligonucleotide is complementary to at least 17 contiguous nucleotides within a miRNA. In some embodiments, the duplexed portion of the oligonucleotide is complementary to at least 18 contiguous nucleotides within a miRNA. In some embodiments, the duplexed portion of the oligonucleotide is complementary to at least 19 contiguous nucleotides within a miRNA. In some embodiments, the duplexed portion of the oligonucleotide is complementary to at least 20 contiguous nucleotides within a miRNA. In some embodiments, the duplexed portion of the oligonucleotide is complementary to at least 21 contiguous nucleotides within a miRNA. In some embodiments, the duplexed portion of the oligonucleotide is complementary to at least 22 contiguous nucleotides within a miRNA. In some embodiments, the duplexed portion of the oligonucleotide is complementary to at least 23 contiguous nucleotides within a miRNA. In some embodiments, the duplexed portion of the oligonucleotide is complementary to at least 24 contiguous nucleotides within a miRNA. In some embodiments, the duplexed portion of the oligonucleotide is complementary to at least 25 contiguous nucleotides within a miRNA. In some embodiments, the duplexed portion of the oligonucleotide is complementary to at least 26 contiguous nucleotides within a miRNA. In some embodiments, the duplexed portion of the oligonucleotide is complementary to at least 27 contiguous nucleotides within a miRNA. In some embodiments, the duplexed portion of the oligonucleotide is complementary to at least 28 contiguous nucleotides within a miRNA. In some embodiments, the duplexed portion of the oligonucleotide is complementary to at least 29 contiguous nucleotides within a miRNA. In some embodiments, the duplexed portion of the oligonucleotide is complementary to at least 30 contiguous nucleotides within a miRNA. In some embodiments, the duplexed portion of the oligonucleotide is at least 50%, 60%, 70%, 75%, 80%, 85%, 90%, 95%, or at least 100% complementary to said miRNA. In some embodiments, the duplexed portion of the oligonucleotide is at least 50% complementary to said miRNA. In some embodiments, the duplexed portion of the oligonucleotide is at least 60% complementary to said miRNA. In some embodiments, the duplexed portion of the oligonucleotide is at least 70% complementary to said miRNA. In some embodiments, the duplexed portion of the oligonucleotide is at least 75% complementary to said miRNA. In some embodiments, the duplexed portion of the oligonucleotide is at least 80% complementary to said miRNA. In some embodiments, the duplexed portion of the oligonucleotide is at least 85% complementary to said miRNA. In some embodiments, the duplexed portion of the oligonucleotide is at least 90% complementary to said miRNA. In some embodiments, the duplexed portion of the oligonucleotide is at least 95% complementary to said miRNA. In a preferred embodiment, the duplexed portion of the oligonucleotide is at least 100% complementary to said miRNA. In some embodiments, duplexed portion of the oligonucleotide comprises between 0 and 5 mismatched base pairs. In some embodiments, duplexed portion of the oligonucleotide comprises less than 5 mismatched base pairs. In some embodiments, duplexed portion of the oligonucleotide comprises less than 4 mismatched base pairs. In some embodiments, duplexed portion of the oligonucleotide comprises less than 3 mismatched base pairs. In some embodiments, duplexed portion of the oligonucleotide comprises less than 2 mismatched base pairs. In some embodiments, duplexed portion of the oligonucleotide comprises less than 1 mismatched base pairs. In a preferred embodiment, the duplexed portion of the oligonucleotide does not comprise mismatched base pairs.

In some embodiments of the methods and compositions disclosed herein, the single-stranded 5′ uncapped triphosphate or biphosphate modified RNA oligonucleotide is capable of forming a duplex with a miRNA, and competes with endogenous mRNA to bind said miRNA. In some embodiments, the duplex is not cleaved by AGO2. In some embodiments, the duplex activates RIG-I. In some embodiments, the RIG-I activation is at least 5%, 10%, 15%, 20%, 25%, 30%, 35%, 40%, 45%, 50%, 55%, 60%, 65%, 70%, 75%, 80%, 85%. 90%, 95%, 100%, 110%, 120%, 130%, 140%, 150%, or 200% greater than activation by a corresponding unmodified monophosphate RNA oligonucleotide. In some embodiments, the RIG-I activation is at least 20% greater than activation by a corresponding unmodified monophosphate RNA oligonucleotide. In some embodiments, the RIG-I activation is at least 25% greater than activation by a corresponding unmodified monophosphate RNA oligonucleotide. In some embodiments, the RIG-I activation is at least 30% greater than activation by a corresponding unmodified monophosphate RNA oligonucleotide. In some embodiments, the RIG-I activation is at least 35% greater than activation by a corresponding unmodified monophosphate RNA oligonucleotide. In some embodiments, the RIG-I activation is at least 40% greater than activation by a corresponding unmodified monophosphate RNA oligonucleotide. In some embodiments, the RIG-I activation is at least 45% greater than activation by a corresponding unmodified monophosphate RNA oligonucleotide. In some embodiments, the RIG-I activation is at least 45% greater than activation by a corresponding unmodified monophosphate RNA oligonucleotide. In some embodiments, the RIG-I activation is at least 50% greater than activation by a corresponding unmodified monophosphate RNA oligonucleotide. In some embodiments, the RIG-I activation is at least 55% greater than activation by a corresponding unmodified monophosphate RNA oligonucleotide. In some embodiments, the RIG-I activation is at least 60% greater than activation by a corresponding unmodified monophosphate RNA oligonucleotide. In some embodiments, the RIG-I activation is at least 65% greater than activation by a corresponding unmodified monophosphate RNA oligonucleotide. In some embodiments, the RIG-I activation is at least 70% greater than activation by a corresponding unmodified monophosphate RNA oligonucleotide. In some embodiments, the RIG-I activation is at least 75% greater than activation by a corresponding unmodified monophosphate RNA oligonucleotide. In some embodiments, the RIG-I activation is at least 80% greater than activation by a corresponding unmodified monophosphate RNA oligonucleotide. In some embodiments, the RIG-I activation is at least 85% greater than activation by a corresponding unmodified monophosphate RNA oligonucleotide. In some embodiments, the RIG-I activation is at least 90% greater than activation by a corresponding unmodified monophosphate RNA oligonucleotide. In some embodiments, the RIG-I activation is at least 95% greater than activation by a corresponding unmodified monophosphate RNA oligonucleotide. In some embodiments, the RIG-I activation is at least 100% greater than activation by a corresponding unmodified monophosphate RNA oligonucleotide. In some embodiments, the RIG-I activation is at least 110% greater than activation by a corresponding unmodified monophosphate RNA oligonucleotide. In some embodiments, the RIG-I activation is at least 120% greater than activation by a corresponding unmodified monophosphate RNA oligonucleotide. In some embodiments, the RIG-I activation is at least 130% greater than activation by a corresponding unmodified monophosphate RNA oligonucleotide. In some embodiments, the RIG-I activation is at least 140% greater than activation by a corresponding unmodified monophosphate RNA oligonucleotide. In some embodiments, the RIG-I activation is at least 150% greater than activation by a corresponding unmodified monophosphate RNA oligonucleotide. In some embodiments, the RIG-I activation is at least 200% greater than activation by a corresponding unmodified monophosphate RNA oligonucleotide. In preferred embodiments, the RIG-I activation elicits a tumor-specific immune response.

The oligonucleotides of the methods and compositions provided herein may comprise modifications. As disclosed herein, modifications may include chemical modification, addition, deletion, substitution, or manipulation of the nucleic acid phosphate backbone, nucleic acid sugar, nucleic acid base, and/or the 5′ or 3′ end of the oligonucleotide. Oligonucleotides, especially those implemented in or as therapeutics, are generally modified on the phosphate backbone and/or ribose sugars to increase nuclease resistance and enhance affinity for target RNAs. A phosphorothioate (PS) backbone modification replaces a non-bridging oxygen atom with a sulfur atom and extends half-life of oligonucleotides in plasma from minutes to days. Enhanced protein binding has also been reported for oligonucleotides with PS-modifications compared to those with phosphodiester (PO) linkages. Further improvement of nuclease stability and binding affinity to target RNAs of oligonucleotides may be obtained by 2′ ribose modifications such as 2′-O-methyl, 2′-fluoro (2′-F), 2′-O-methoxyethyl (2′-MOE), 2′,4′-constrained 2′-O-ethyl (cEt) and locked nucleic acid (LNA). The positions of 2′ modifications within an oligonucleotide sequence can further influence protein-oligonucleotide interactions. In certain embodiments, the disclosure provides methods and compositions comprising single-stranded 5′ uncapped triphosphate or biphosphate modified RNA oligonucleotides, wherein said oligonucleotide is complementary to an endogenous tumor-specific RNA which is highly expressed in tumor cells in comparison to non-tumor cells. In certain embodiments, the disclosure provides methods and compositions comprising a single-stranded 5′ uncapped triphosphate or biphosphate modified RNA oligonucleotide, wherein said single-stranded 5′ uncapped triphosphate or biphosphate modified RNA oligonucleotide comprises a sequence which is complementary to an endogenous tumor-specific RNA. In some embodiments, the single-stranded 5′ uncapped triphosphate or biphosphate modified RNA oligonucleotide comprises other modifications. In some embodiments, the single-stranded 5′ uncapped triphosphate or biphosphate modified RNA oligonucleotide does not comprise any other modifications.

In some embodiments, the single-stranded 5′ uncapped triphosphate or biphosphate modified RNA oligonucleotide further comprises a 2′-fluoro (2′-F) ribose modification. In some embodiments, the 2′-F ribose modification is present when the corresponding base is a cytosine or a uracil. In some embodiments, the 2′-F ribose modification is present at the 10^{th} or 11^{th} nucleotide from the 5′-terminus of the modified RNA oligonucleotide. In some embodiments, the single-stranded 5′ uncapped triphosphate or biphosphate modified RNA oligonucleotide further comprises a phosphorothioate (PS) backbone modification. In some embodiments, the single-stranded 5′ uncapped triphosphate or biphosphate modified RNA oligonucleotide further comprises a 2′-fluoro (2′-F) ribose modification and a phosphorothioate (PS) backbone modification.

In a preferred embodiment, the single-stranded 5′ uncapped triphosphate or biphosphate modified RNA oligonucleotide does not comprise any other modifications. In a preferred embodiment, the single-stranded 5′ uncapped triphosphate or biphosphate modified RNA oligonucleotide does not comprise any other modifications selected from the group consisting of: 2′-O-methyl (2′-OMe) ribose modification, N-6-methyladenosine (m6A), pseudouridine (Ψ), N-1-methylpseudouridine (mΨ), N-1-methylpseudouridine (mΨ), 5-methyl-cytidine (5mC), 5-hydroxymethyl-cytidine (5hmC), or 5-methoxycytidine (5moC). In some embodiments the single-stranded 5′ uncapped triphosphate or biphosphate modified RNA oligonucleotide does not comprise 2′-O-methyl (2′-OMe) ribose modification. In some embodiments, the single-stranded 5′ uncapped triphosphate or biphosphate modified RNA oligonucleotide does not comprise a N-6-methyladenosine (m6A). In some embodiments, the single-stranded 5′ uncapped triphosphate or biphosphate modified RNA oligonucleotide does not comprise a pseudouridine (Ψ). In some embodiments, the single-stranded 5′ uncapped triphosphate or biphosphate modified RNA oligonucleotide does not comprise a N-1-methylpseudouridine (mΨ). In some embodiments, the single-stranded 5′ uncapped triphosphate or biphosphate modified RNA oligonucleotide does not comprise a 5-methyl-cytidine (5mC). In some embodiments, the single-stranded 5′ uncapped triphosphate or biphosphate modified RNA oligonucleotide does not comprise a 5-hydroxymethyl-cytidine (5hmC). In some embodiments, the single-stranded 5′ uncapped triphosphate or biphosphate modified RNA oligonucleotide does not comprise a 5-methoxycytidine (5moC).

In some embodiments the single-stranded 5′ uncapped triphosphate or biphosphate modified RNA oligonucleotide comprises one or more modifications. In some embodiments, the single-stranded 5′ uncapped triphosphate or biphosphate modified RNA oligonucleotide comprises one or more modifications selected from the group consisting of: phosphorothioate (PS) backbone modification, 2′-O-methyl (2′-OMe) ribose modification, N-6-methyladenosine (m6A), pseudouridine (Ψ), N-1-methylpseudouridine (mΨ), N-1-methylpseudouridine (mΨ), 5-methyl-cytidine (5mC), 5-hydroxymethyl-cytidine (5hmC), or 5-methoxycytidine (5moC). In some embodiments the single-stranded 5′ uncapped triphosphate or biphosphate modified RNA oligonucleotide comprises 2′-O-methyl (2′-OMe) ribose modification. In some embodiments, the single-stranded 5′ uncapped triphosphate or biphosphate modified RNA oligonucleotide comprises a N-6-methyladenosine (m6A). In some embodiments, the single-stranded 5′ uncapped triphosphate or biphosphate modified RNA oligonucleotide comprises a pseudouridine (Ψ). In some embodiments, the single-stranded 5′ uncapped triphosphate or biphosphate modified RNA oligonucleotide comprises a N-1-methylpseudouridine (mΨ). In some embodiments, the single-stranded 5′ uncapped triphosphate or biphosphate modified RNA oligonucleotide comprises a 5-methyl-cytidine (5mC). In some embodiments, the single-stranded 5′ uncapped triphosphate or biphosphate modified RNA oligonucleotide comprises a 5-hydroxymethyl-cytidine (5hmC). In some embodiments, the single-stranded 5′ uncapped triphosphate or biphosphate modified RNA oligonucleotide does comprises 5-methoxycytidine (5moC).

In some embodiments of the methods and compositions disclosed herein, the single-stranded 5′ uncapped triphosphate or biphosphate modified RNA oligonucleotide comprises a sequence which is at least 10 nucleotides in length. In some embodiments, the oligonucleotide comprises a sequence which is at least 15 nucleotides in length. In some embodiments, the oligonucleotide comprises a sequence which is at least 16 nucleotides in length. In some embodiments, the oligonucleotide comprises a sequence which is at least 17 nucleotides in length. In some embodiments, the oligonucleotide comprises a sequence which is at least 18 nucleotides in length. In some embodiments, the oligonucleotide comprises a sequence which is at least 19 nucleotides in length. In some embodiments, the oligonucleotide comprises a sequence which is at least 20 nucleotides in length. In some embodiments, the oligonucleotide comprises a sequence which is at least 21 nucleotides in length. In some embodiments, the oligonucleotide comprises a sequence which is at least 22 nucleotides in length. In some embodiments, the oligonucleotide comprises a sequence which is at least 23 nucleotides in length. In some embodiments, the oligonucleotide comprises a sequence which is at least 24 nucleotides in length. In some embodiments, the oligonucleotide comprises a sequence which is at least 25 nucleotides in length. In some embodiments, the oligonucleotide comprises a sequence which is at least 26 nucleotides in length. In some embodiments, the oligonucleotide comprises a sequence which is at least 27 nucleotides in length. In some embodiments, the oligonucleotide comprises a sequence which is at least 28 nucleotides in length. In some embodiments, the oligonucleotide comprises a sequence which is at least 29 nucleotides in length. In some embodiments, the oligonucleotide comprises a sequence which is at least 30 nucleotides in length. In some embodiments, the oligonucleotide comprises a sequence which is at least 50 nucleotides in length. In some embodiments, the oligonucleotide comprises a sequence which is between 15 and 50 nucleotides in length. In some embodiments, the oligonucleotide comprises a sequence which is between 15 and 30 nucleotides in length. In some embodiments, the oligonucleotide comprises a sequence which is between 15 and 29 nucleotides in length. In some embodiments, the oligonucleotide comprises a sequence which is between 15 and 28 nucleotides in length. In some embodiments, the oligonucleotide comprises a sequence which is between 15 and 27 nucleotides in length. In some embodiments, the oligonucleotide comprises a sequence which is between 15 and 26 nucleotides in length. In some embodiments, the oligonucleotide comprises a sequence which is between 15 and 25 nucleotides in length. In some embodiments, the oligonucleotide comprises a sequence which is between 16 and 50 nucleotides in length. In some embodiments, the oligonucleotide comprises a sequence which is between 16 and 30 nucleotides in length. In some embodiments, the oligonucleotide comprises a sequence which is between 16 and 29 nucleotides in length. In some embodiments, the oligonucleotide comprises a sequence which is between 16 and 28 nucleotides in length. In some embodiments, the oligonucleotide comprises a sequence which is between 16 and 27 nucleotides in length. In some embodiments, the oligonucleotide comprises a sequence which is between 16 and 26 nucleotides in length. In some embodiments, the oligonucleotide comprises a sequence which is between 16 and 25 nucleotides in length.

The 5′pp and 5′ppp anti-miRNAs/mRNAs comprise sequences that are complementary to at least 10 (e.g., at least 11, 12, 13, 14, 15, 16, 17, 18, 19, 20, 21, 22, or 23) contiguous nucleotides within a miRNA or mRNA. Exemplary miRNAs include, e.g., miR-9; miR-10b; miR-21; miR-106b/93; miR-125b; miR-130a; miR-155; miR-181a; miR-200s; miR-210-3p; miR-221/222; miR-335; miR-498; miR-504; miR-1810; miR-1908; miR-224/452; or miR-181/340 (see, e.g., Table 1 of Nguyen and Chang, Int J Mol Sci. 2017; 19(1):65) and those listed in Table 1 and Table 2 herein. Exemplary mRNAs include those listed in Table 2 herein.

In certain embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 80% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 85% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 90% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 95% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 97% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 98% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is at least 99% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that is 100% identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide comprises a nucleic acid sequence that identical to a nucleic acid sequence selected from SEQ ID NOs: 1, 2, 3, 4, 5, 6, 7, 8, 9, 10, 11, 12, or 13. In some embodiments, the single-stranded 5′ uncapped triphosphate or biphosphate modified RNA oligonucleotide consists of a nucleic acid sequence that is identical to a nucleic acid sequence selected from SEQ ID NOs: 1, 2, 3, 4, 5, 6, 7, 8, 9, 10, 11, 12, or 13.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 1. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 1. In some embodiments, the single-stranded 5′ uncapped triphosphate or biphosphate modified RNA oligonucleotide comprises a nucleic acid sequence that is at least 80% identical to the nucleic acid sequence of SEQ ID NO: 1. In some embodiments, the single-stranded 5′ uncapped triphosphate or biphosphate modified RNA oligonucleotide comprises a nucleic acid sequence that is at least 85% identical to the nucleic acid sequence of SEQ ID NO: 1. In some embodiments, the single-stranded 5′ uncapped triphosphate or biphosphate modified RNA oligonucleotide comprises a nucleic acid sequence that is at least 90% identical to the nucleic acid sequence of SEQ ID NO: 1. In some embodiments, the single-stranded 5′ uncapped triphosphate or biphosphate modified RNA oligonucleotide comprises a nucleic acid sequence that is at least 95% identical to the nucleic acid sequence of SEQ ID NO: 1. In some embodiments, the single-stranded 5′ uncapped triphosphate or biphosphate modified RNA oligonucleotide comprises a nucleic acid sequence that is at least 97% identical to the nucleic acid sequence of SEQ ID NO: 1. In some embodiments, the single-stranded 5′ uncapped triphosphate or biphosphate modified RNA oligonucleotide comprises a nucleic acid sequence that is at least 98% identical to the nucleic acid sequence of SEQ ID NO: 1. In some embodiments, the single-stranded 5′ uncapped triphosphate or biphosphate modified RNA oligonucleotide comprises a nucleic acid sequence that is at least 99% identical to the nucleic acid sequence of SEQ ID NO: 1. In some embodiments, the single-stranded 5′ uncapped triphosphate or biphosphate modified RNA oligonucleotide comprises a nucleic acid sequence that is 100% identical to the nucleic acid sequence of SEQ ID NO: 1. In some embodiments, the single-stranded 5′ uncapped triphosphate or biphosphate modified RNA oligonucleotide comprises a nucleic acid sequence that is identical to the nucleic acid sequence of SEQ ID NO: 1. In some embodiments, the single-stranded 5′ uncapped triphosphate or biphosphate modified RNA oligonucleotide consists of a nucleic acid sequence that is identical to a nucleic acid sequence selected from SEQ ID NO: 1.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 2. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 2. In some embodiments, the single-stranded 5′ uncapped triphosphate or biphosphate modified RNA oligonucleotide comprises a nucleic acid sequence that is at least 80% identical to the nucleic acid sequence of SEQ ID NO: 2. In some embodiments, the single-stranded 5′ uncapped triphosphate or biphosphate modified RNA oligonucleotide comprises a nucleic acid sequence that is at least 85% identical to the nucleic acid sequence of SEQ ID NO: 2. In some embodiments, the single-stranded 5′ uncapped triphosphate or biphosphate modified RNA oligonucleotide comprises a nucleic acid sequence that is at least 90% identical to the nucleic acid sequence of SEQ ID NO: 2. In some embodiments, the single-stranded 5′ uncapped triphosphate or biphosphate modified RNA oligonucleotide comprises a nucleic acid sequence that is at least 95% identical to the nucleic acid sequence of SEQ ID NO: 2. In some embodiments, the single-stranded 5′ uncapped triphosphate or biphosphate modified RNA oligonucleotide comprises a nucleic acid sequence that is at least 97% identical to the nucleic acid sequence of SEQ ID NO: 2. In some embodiments, the single-stranded 5′ uncapped triphosphate or biphosphate modified RNA oligonucleotide comprises a nucleic acid sequence that is at least 98% identical to the nucleic acid sequence of SEQ ID NO: 2. In some embodiments, the single-stranded 5′ uncapped triphosphate or biphosphate modified RNA oligonucleotide comprises a nucleic acid sequence that is at least 99% identical to the nucleic acid sequence of SEQ ID NO: 2. In some embodiments, the single-stranded 5′ uncapped triphosphate or biphosphate modified RNA oligonucleotide comprises a nucleic acid sequence that is 100% identical to the nucleic acid sequence of SEQ ID NO: 2. In some embodiments, the single-stranded 5′ uncapped triphosphate or biphosphate modified RNA oligonucleotide comprises a nucleic acid sequence that is identical to the nucleic acid sequence of SEQ ID NO: 2. In some embodiments, the single-stranded 5′ uncapped triphosphate or biphosphate modified RNA oligonucleotide consists of a nucleic acid sequence that is identical to a nucleic acid sequence selected from SEQ ID NO: 2.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 3. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 3. In some embodiments, the single-stranded 5′ uncapped triphosphate or biphosphate modified RNA oligonucleotide comprises a nucleic acid sequence that is at least 80% identical to the nucleic acid sequence of SEQ ID NO: 3. In some embodiments, the single-stranded 5′ uncapped triphosphate or biphosphate modified RNA oligonucleotide comprises a nucleic acid sequence that is at least 85% identical to the nucleic acid sequence of SEQ ID NO: 3. In some embodiments, the single-stranded 5′ uncapped triphosphate or biphosphate modified RNA oligonucleotide comprises a nucleic acid sequence that is at least 90% identical to the nucleic acid sequence of SEQ ID NO: 3. In some embodiments, the single-stranded 5′ uncapped triphosphate or biphosphate modified RNA oligonucleotide comprises a nucleic acid sequence that is at least 95% identical to the nucleic acid sequence of SEQ ID NO: 3. In some embodiments, the single-stranded 5′ uncapped triphosphate or biphosphate modified RNA oligonucleotide comprises a nucleic acid sequence that is at least 97% identical to the nucleic acid sequence of SEQ ID NO: 3. In some embodiments, the single-stranded 5′ uncapped triphosphate or biphosphate modified RNA oligonucleotide comprises a nucleic acid sequence that is at least 98% identical to the nucleic acid sequence of SEQ ID NO: 3. In some embodiments, the single-stranded 5′ uncapped triphosphate or biphosphate modified RNA oligonucleotide comprises a nucleic acid sequence that is at least 99% identical to the nucleic acid sequence of SEQ ID NO: 3. In some embodiments, the single-stranded 5′ uncapped triphosphate or biphosphate modified RNA oligonucleotide comprises a nucleic acid sequence that is 100% identical to the nucleic acid sequence of SEQ ID NO: 3. In some embodiments, the single-stranded 5′ uncapped triphosphate or biphosphate modified RNA oligonucleotide comprises a nucleic acid sequence that is identical to the nucleic acid sequence of SEQ ID NO: 3. In some embodiments, the single-stranded 5′ uncapped triphosphate or biphosphate modified RNA oligonucleotide consists of a nucleic acid sequence that is identical to a nucleic acid sequence selected from SEQ ID NO: 3.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 4. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 4. In some embodiments, the single-stranded 5′ uncapped triphosphate or biphosphate modified RNA oligonucleotide comprises a nucleic acid sequence that is at least 80% identical to the nucleic acid sequence of SEQ ID NO: 4. In some embodiments, the single-stranded 5′ uncapped triphosphate or biphosphate modified RNA oligonucleotide comprises a nucleic acid sequence that is at least 85% identical to the nucleic acid sequence of SEQ ID NO: 4. In some embodiments, the single-stranded 5′ uncapped triphosphate or biphosphate modified RNA oligonucleotide comprises a nucleic acid sequence that is at least 90% identical to the nucleic acid sequence of SEQ ID NO: 4. In some embodiments, the single-stranded 5′ uncapped triphosphate or biphosphate modified RNA oligonucleotide comprises a nucleic acid sequence that is at least 95% identical to the nucleic acid sequence of SEQ ID NO: 4. In some embodiments, the single-stranded 5′ uncapped triphosphate or biphosphate modified RNA oligonucleotide comprises a nucleic acid sequence that is at least 97% identical to the nucleic acid sequence of SEQ ID NO: 4. In some embodiments, the single-stranded 5′ uncapped triphosphate or biphosphate modified RNA oligonucleotide comprises a nucleic acid sequence that is at least 98% identical to the nucleic acid sequence of SEQ ID NO: 4. In some embodiments, the single-stranded 5′ uncapped triphosphate or biphosphate modified RNA oligonucleotide comprises a nucleic acid sequence that is at least 99% identical to the nucleic acid sequence of SEQ ID NO: 4. In some embodiments, the single-stranded 5′ uncapped triphosphate or biphosphate modified RNA oligonucleotide comprises a nucleic acid sequence that is 100% identical to the nucleic acid sequence of SEQ ID NO: 4. In some embodiments, the single-stranded 5′ uncapped triphosphate or biphosphate modified RNA oligonucleotide comprises a nucleic acid sequence that is identical to the nucleic acid sequence of SEQ ID NO: 4. In some embodiments, the single-stranded 5′ uncapped triphosphate or biphosphate modified RNA oligonucleotide consists of a nucleic acid sequence that is identical to a nucleic acid sequence selected from SEQ ID NO: 4.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 5. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 5. In some embodiments, the single-stranded 5′ uncapped triphosphate or biphosphate modified RNA oligonucleotide comprises a nucleic acid sequence that is at least 80% identical to the nucleic acid sequence of SEQ ID NO: 5. In some embodiments, the single-stranded 5′ uncapped triphosphate or biphosphate modified RNA oligonucleotide comprises a nucleic acid sequence that is at least 85% identical to the nucleic acid sequence of SEQ ID NO: 5. In some embodiments, the single-stranded 5′ uncapped triphosphate or biphosphate modified RNA oligonucleotide comprises a nucleic acid sequence that is at least 90% identical to the nucleic acid sequence of SEQ ID NO: 5. In some embodiments, the single-stranded 5′ uncapped triphosphate or biphosphate modified RNA oligonucleotide comprises a nucleic acid sequence that is at least 95% identical to the nucleic acid sequence of SEQ ID NO: 5. In some embodiments, the single-stranded 5′ uncapped triphosphate or biphosphate modified RNA oligonucleotide comprises a nucleic acid sequence that is at least 97% identical to the nucleic acid sequence of SEQ ID NO: 5. In some embodiments, the single-stranded 5′ uncapped triphosphate or biphosphate modified RNA oligonucleotide comprises a nucleic acid sequence that is at least 98% identical to the nucleic acid sequence of SEQ ID NO: 5. In some embodiments, the single-stranded 5′ uncapped triphosphate or biphosphate modified RNA oligonucleotide comprises a nucleic acid sequence that is at least 99% identical to the nucleic acid sequence of SEQ ID NO: 5. In some embodiments, the single-stranded 5′ uncapped triphosphate or biphosphate modified RNA oligonucleotide comprises a nucleic acid sequence that is 100% identical to the nucleic acid sequence of SEQ ID NO: 5. In some embodiments, the single-stranded 5′ uncapped triphosphate or biphosphate modified RNA oligonucleotide comprises a nucleic acid sequence that is identical to the nucleic acid sequence of SEQ ID NO: 5. In some embodiments, the single-stranded 5′ uncapped triphosphate or biphosphate modified RNA oligonucleotide consists of a nucleic acid sequence that is identical to a nucleic acid sequence selected from SEQ ID NO: 5.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 1. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 6. In some embodiments, the single-stranded 5′ uncapped triphosphate or biphosphate modified RNA oligonucleotide comprises a nucleic acid sequence that is at least 80% identical to the nucleic acid sequence of SEQ ID NO: 6. In some embodiments, the single-stranded 5′ uncapped triphosphate or biphosphate modified RNA oligonucleotide comprises a nucleic acid sequence that is at least 85% identical to the nucleic acid sequence of SEQ ID NO: 6. In some embodiments, the single-stranded 5′ uncapped triphosphate or biphosphate modified RNA oligonucleotide comprises a nucleic acid sequence that is at least 90% identical to the nucleic acid sequence of SEQ ID NO: 6. In some embodiments, the single-stranded 5′ uncapped triphosphate or biphosphate modified RNA oligonucleotide comprises a nucleic acid sequence that is at least 95% identical to the nucleic acid sequence of SEQ ID NO: 6. In some embodiments, the single-stranded 5′ uncapped triphosphate or biphosphate modified RNA oligonucleotide comprises a nucleic acid sequence that is at least 97% identical to the nucleic acid sequence of SEQ ID NO: 6. In some embodiments, the single-stranded 5′ uncapped triphosphate or biphosphate modified RNA oligonucleotide comprises a nucleic acid sequence that is at least 98% identical to the nucleic acid sequence of SEQ ID NO: 6. In some embodiments, the single-stranded 5′ uncapped triphosphate or biphosphate modified RNA oligonucleotide comprises a nucleic acid sequence that is at least 99% identical to the nucleic acid sequence of SEQ ID NO: 6. In some embodiments, the single-stranded 5′ uncapped triphosphate or biphosphate modified RNA oligonucleotide comprises a nucleic acid sequence that is 100% identical to the nucleic acid sequence of SEQ ID NO: 6. In some embodiments, the single-stranded 5′ uncapped triphosphate or biphosphate modified RNA oligonucleotide comprises a nucleic acid sequence that is identical to the nucleic acid sequence of SEQ ID NO: 6. In some embodiments, the single-stranded 5′ uncapped triphosphate or biphosphate modified RNA oligonucleotide consists of a nucleic acid sequence that is identical to a nucleic acid sequence selected from SEQ ID NO: 6.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 7. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 7. In some embodiments, the single-stranded 5′ uncapped triphosphate or biphosphate modified RNA oligonucleotide comprises a nucleic acid sequence that is at least 80% identical to the nucleic acid sequence of SEQ ID NO: 7. In some embodiments, the single-stranded 5′ uncapped triphosphate or biphosphate modified RNA oligonucleotide comprises a nucleic acid sequence that is at least 85% identical to the nucleic acid sequence of SEQ ID NO: 7. In some embodiments, the single-stranded 5′ uncapped triphosphate or biphosphate modified RNA oligonucleotide comprises a nucleic acid sequence that is at least 90% identical to the nucleic acid sequence of SEQ ID NO: 7. In some embodiments, the single-stranded 5′ uncapped triphosphate or biphosphate modified RNA oligonucleotide comprises a nucleic acid sequence that is at least 95% identical to the nucleic acid sequence of SEQ ID NO: 7. In some embodiments, the single-stranded 5′ uncapped triphosphate or biphosphate modified RNA oligonucleotide comprises a nucleic acid sequence that is at least 97% identical to the nucleic acid sequence of SEQ ID NO: 7. In some embodiments, the single-stranded 5′ uncapped triphosphate or biphosphate modified RNA oligonucleotide comprises a nucleic acid sequence that is at least 98% identical to the nucleic acid sequence of SEQ ID NO: 7. In some embodiments, the single-stranded 5′ uncapped triphosphate or biphosphate modified RNA oligonucleotide comprises a nucleic acid sequence that is at least 99% identical to the nucleic acid sequence of SEQ ID NO: 7. In some embodiments, the single-stranded 5′ uncapped triphosphate or biphosphate modified RNA oligonucleotide comprises a nucleic acid sequence that is 100% identical to the nucleic acid sequence of SEQ ID NO: 7. In some embodiments, the single-stranded 5′ uncapped triphosphate or biphosphate modified RNA oligonucleotide comprises a nucleic acid sequence that is identical to the nucleic acid sequence of SEQ ID NO: 7. In some embodiments, the single-stranded 5′ uncapped triphosphate or biphosphate modified RNA oligonucleotide consists of a nucleic acid sequence that is identical to a nucleic acid sequence selected from SEQ ID NO: 7.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 8. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 8. In some embodiments, the single-stranded 5′ uncapped triphosphate or biphosphate modified RNA oligonucleotide comprises a nucleic acid sequence that is at least 80% identical to the nucleic acid sequence of SEQ ID NO: 8. In some embodiments, the single-stranded 5′ uncapped triphosphate or biphosphate modified RNA oligonucleotide comprises a nucleic acid sequence that is at least 85% identical to the nucleic acid sequence of SEQ ID NO: 8. In some embodiments, the single-stranded 5′ uncapped triphosphate or biphosphate modified RNA oligonucleotide comprises a nucleic acid sequence that is at least 90% identical to the nucleic acid sequence of SEQ ID NO: 8. In some embodiments, the single-stranded 5′ uncapped triphosphate or biphosphate modified RNA oligonucleotide comprises a nucleic acid sequence that is at least 95% identical to the nucleic acid sequence of SEQ ID NO: 8. In some embodiments, the single-stranded 5′ uncapped triphosphate or biphosphate modified RNA oligonucleotide comprises a nucleic acid sequence that is at least 97% identical to the nucleic acid sequence of SEQ ID NO: 8. In some embodiments, the single-stranded 5′ uncapped triphosphate or biphosphate modified RNA oligonucleotide comprises a nucleic acid sequence that is at least 98% identical to the nucleic acid sequence of SEQ ID NO: 8. In some embodiments, the single-stranded 5′ uncapped triphosphate or biphosphate modified RNA oligonucleotide comprises a nucleic acid sequence that is at least 99% identical to the nucleic acid sequence of SEQ ID NO: 8. In some embodiments, the single-stranded 5′ uncapped triphosphate or biphosphate modified RNA oligonucleotide comprises a nucleic acid sequence that is 100% identical to the nucleic acid sequence of SEQ ID NO: 8. In some embodiments, the single-stranded 5′ uncapped triphosphate or biphosphate modified RNA oligonucleotide comprises a nucleic acid sequence that is identical to the nucleic acid sequence of SEQ ID NO: 8. In some embodiments, the single-stranded 5′ uncapped triphosphate or biphosphate modified RNA oligonucleotide consists of a nucleic acid sequence that is identical to a nucleic acid sequence selected from SEQ ID NO: 8.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 9. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 9. In some embodiments, the single-stranded 5′ uncapped triphosphate or biphosphate modified RNA oligonucleotide comprises a nucleic acid sequence that is at least 80% identical to the nucleic acid sequence of SEQ ID NO: 9. In some embodiments, the single-stranded 5′ uncapped triphosphate or biphosphate modified RNA oligonucleotide comprises a nucleic acid sequence that is at least 85% identical to the nucleic acid sequence of SEQ ID NO: 9. In some embodiments, the single-stranded 5′ uncapped triphosphate or biphosphate modified RNA oligonucleotide comprises a nucleic acid sequence that is at least 90% identical to the nucleic acid sequence of SEQ ID NO: 9. In some embodiments, the single-stranded 5′ uncapped triphosphate or biphosphate modified RNA oligonucleotide comprises a nucleic acid sequence that is at least 95% identical to the nucleic acid sequence of SEQ ID NO: 9. In some embodiments, the single-stranded 5′ uncapped triphosphate or biphosphate modified RNA oligonucleotide comprises a nucleic acid sequence that is at least 97% identical to the nucleic acid sequence of SEQ ID NO: 9. In some embodiments, the single-stranded 5′ uncapped triphosphate or biphosphate modified RNA oligonucleotide comprises a nucleic acid sequence that is at least 98% identical to the nucleic acid sequence of SEQ ID NO: 9. In some embodiments, the single-stranded 5′ uncapped triphosphate or biphosphate modified RNA oligonucleotide comprises a nucleic acid sequence that is at least 99% identical to the nucleic acid sequence of SEQ ID NO: 9. In some embodiments, the single-stranded 5′ uncapped triphosphate or biphosphate modified RNA oligonucleotide comprises a nucleic acid sequence that is 100% identical to the nucleic acid sequence of SEQ ID NO: 9. In some embodiments, the single-stranded 5′ uncapped triphosphate or biphosphate modified RNA oligonucleotide comprises a nucleic acid sequence that is identical to the nucleic acid sequence of SEQ ID NO: 9. In some embodiments, the single-stranded 5′ uncapped triphosphate or biphosphate modified RNA oligonucleotide consists of a nucleic acid sequence that is identical to a nucleic acid sequence selected from SEQ ID NO: 9.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 10. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 10. In some embodiments, the single-stranded 5′ uncapped triphosphate or biphosphate modified RNA oligonucleotide comprises a nucleic acid sequence that is at least 80% identical to the nucleic acid sequence of SEQ ID NO: 10. In some embodiments, the single-stranded 5′ uncapped triphosphate or biphosphate modified RNA oligonucleotide comprises a nucleic acid sequence that is at least 85% identical to the nucleic acid sequence of SEQ ID NO: 10. In some embodiments, the single-stranded 5′ uncapped triphosphate or biphosphate modified RNA oligonucleotide comprises a nucleic acid sequence that is at least 90% identical to the nucleic acid sequence of SEQ ID NO: 10. In some embodiments, the single-stranded 5′ uncapped triphosphate or biphosphate modified RNA oligonucleotide comprises a nucleic acid sequence that is at least 95% identical to the nucleic acid sequence of SEQ ID NO: 10. In some embodiments, the single-stranded 5′ uncapped triphosphate or biphosphate modified RNA oligonucleotide comprises a nucleic acid sequence that is at least 97% identical to the nucleic acid sequence of SEQ ID NO: 10. In some embodiments, the single-stranded 5′ uncapped triphosphate or biphosphate modified RNA oligonucleotide comprises a nucleic acid sequence that is at least 98% identical to the nucleic acid sequence of SEQ ID NO: 10. In some embodiments, the single-stranded 5′ uncapped triphosphate or biphosphate modified RNA oligonucleotide comprises a nucleic acid sequence that is at least 99% identical to the nucleic acid sequence of SEQ ID NO: 10. In some embodiments, the single-stranded 5′ uncapped triphosphate or biphosphate modified RNA oligonucleotide comprises a nucleic acid sequence that is 100% identical to the nucleic acid sequence of SEQ ID NO: 10. In some embodiments, the single-stranded 5′ uncapped triphosphate or biphosphate modified RNA oligonucleotide comprises a nucleic acid sequence that is identical to the nucleic acid sequence of SEQ ID NO: 10. In some embodiments, the single-stranded 5′ uncapped triphosphate or biphosphate modified RNA oligonucleotide consists of a nucleic acid sequence that is identical to a nucleic acid sequence selected from SEQ ID NO: 10.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 11. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 11. In some embodiments, the single-stranded 5′ uncapped triphosphate or biphosphate modified RNA oligonucleotide comprises a nucleic acid sequence that is at least 80% identical to the nucleic acid sequence of SEQ ID NO: 11. In some embodiments, the single-stranded 5′ uncapped triphosphate or biphosphate modified RNA oligonucleotide comprises a nucleic acid sequence that is at least 85% identical to the nucleic acid sequence of SEQ ID NO: 11. In some embodiments, the single-stranded 5′ uncapped triphosphate or biphosphate modified RNA oligonucleotide comprises a nucleic acid sequence that is at least 90% identical to the nucleic acid sequence of SEQ ID NO: 11. In some embodiments, the single-stranded 5′ uncapped triphosphate or biphosphate modified RNA oligonucleotide comprises a nucleic acid sequence that is at least 95% identical to the nucleic acid sequence of SEQ ID NO: 11. In some embodiments, the single-stranded 5′ uncapped triphosphate or biphosphate modified RNA oligonucleotide comprises a nucleic acid sequence that is at least 97% identical to the nucleic acid sequence of SEQ ID NO: 11. In some embodiments, the single-stranded 5′ uncapped triphosphate or biphosphate modified RNA oligonucleotide comprises a nucleic acid sequence that is at least 98% identical to the nucleic acid sequence of SEQ ID NO: 11. In some embodiments, the single-stranded 5′ uncapped triphosphate or biphosphate modified RNA oligonucleotide comprises a nucleic acid sequence that is at least 99% identical to the nucleic acid sequence of SEQ ID NO: 11. In some embodiments, the single-stranded 5′ uncapped triphosphate or biphosphate modified RNA oligonucleotide comprises a nucleic acid sequence that is 100% identical to the nucleic acid sequence of SEQ ID NO: 11. In some embodiments, the single-stranded 5′ uncapped triphosphate or biphosphate modified RNA oligonucleotide comprises a nucleic acid sequence that is identical to the nucleic acid sequence of SEQ ID NO: 11. In some embodiments, the single-stranded 5′ uncapped triphosphate or biphosphate modified RNA oligonucleotide consists of a nucleic acid sequence that is identical to a nucleic acid sequence selected from SEQ ID NO: 11.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 12. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 12. In some embodiments, the single-stranded 5′ uncapped triphosphate or biphosphate modified RNA oligonucleotide comprises a nucleic acid sequence that is at least 80% identical to the nucleic acid sequence of SEQ ID NO: 12. In some embodiments, the single-stranded 5′ uncapped triphosphate or biphosphate modified RNA oligonucleotide comprises a nucleic acid sequence that is at least 85% identical to the nucleic acid sequence of SEQ ID NO: 12. In some embodiments, the single-stranded 5′ uncapped triphosphate or biphosphate modified RNA oligonucleotide comprises a nucleic acid sequence that is at least 90% identical to the nucleic acid sequence of SEQ ID NO: 12. In some embodiments, the single-stranded 5′ uncapped triphosphate or biphosphate modified RNA oligonucleotide comprises a nucleic acid sequence that is at least 95% identical to the nucleic acid sequence of SEQ ID NO: 12. In some embodiments, the single-stranded 5′ uncapped triphosphate or biphosphate modified RNA oligonucleotide comprises a nucleic acid sequence that is at least 97% identical to the nucleic acid sequence of SEQ ID NO: 12. In some embodiments, the single-stranded 5′ uncapped triphosphate or biphosphate modified RNA oligonucleotide comprises a nucleic acid sequence that is at least 98% identical to the nucleic acid sequence of SEQ ID NO: 12. In some embodiments, the single-stranded 5′ uncapped triphosphate or biphosphate modified RNA oligonucleotide comprises a nucleic acid sequence that is at least 99% identical to the nucleic acid sequence of SEQ ID NO: 12. In some embodiments, the single-stranded 5′ uncapped triphosphate or biphosphate modified RNA oligonucleotide comprises a nucleic acid sequence that is 100% identical to the nucleic acid sequence of SEQ ID NO: 12. In some embodiments, the single-stranded 5′ uncapped triphosphate or biphosphate modified RNA oligonucleotide comprises a nucleic acid sequence that is identical to the nucleic acid sequence of SEQ ID NO: 12. In some embodiments, the single-stranded 5′ uncapped triphosphate or biphosphate modified RNA oligonucleotide consists of a nucleic acid sequence that is identical to a nucleic acid sequence selected from SEQ ID NO: 12.

In some embodiments, the methods and compositions of the present disclosure comprise a single-stranded 5′ uncapped triphosphate modified RNA oligonucleotide. In certain embodiments, the methods and compositions of the present disclosure comprise a single-stranded 5′ uncapped biphosphate modified RNA oligonucleotide. In a preferred embodiment of the present disclosure, the single-stranded 5′ uncapped triphosphate or biphosphate modified RNA oligonucleotide comprises a nucleic acid sequence that is at least 75% identical to the nucleic acid sequence of SEQ ID NO: 13. In some embodiments, the single-stranded 5′ uncapped triphosphate or biphosphate modified RNA oligonucleotide comprises a nucleic acid sequence that is at least 80%, 85%, 90%, 95%, 97%, 98%, 99% or 100% identical to the nucleic acid sequence of SEQ ID NO: 13. In some embodiments, the single-stranded 5′ uncapped triphosphate or biphosphate modified RNA oligonucleotide comprises a nucleic acid sequence that is at least 80% identical to the nucleic acid sequence of SEQ ID NO: 13. In some embodiments, the single-stranded 5′ uncapped triphosphate or biphosphate modified RNA oligonucleotide comprises a nucleic acid sequence that is at least 85% identical to the nucleic acid sequence of SEQ ID NO: 13. In some embodiments, the single-stranded 5′ uncapped triphosphate or biphosphate modified RNA oligonucleotide comprises a nucleic acid sequence that is at least 90% identical to the nucleic acid sequence of SEQ ID NO: 13. In some embodiments, the single-stranded 5′ uncapped triphosphate or biphosphate modified RNA oligonucleotide comprises a nucleic acid sequence that is at least 95% identical to the nucleic acid sequence of SEQ ID NO: 13. In some embodiments, the single-stranded 5′ uncapped triphosphate or biphosphate modified RNA oligonucleotide comprises a nucleic acid sequence that is at least 97% identical to the nucleic acid sequence of SEQ ID NO: 13. In some embodiments, the single-stranded 5′ uncapped triphosphate or biphosphate modified RNA oligonucleotide comprises a nucleic acid sequence that is at least 98% identical to the nucleic acid sequence of SEQ ID NO: 13. In some embodiments, the single-stranded 5′ uncapped triphosphate or biphosphate modified RNA oligonucleotide comprises a nucleic acid sequence that is at least 99% identical to the nucleic acid sequence of SEQ ID NO: 13. In some embodiments, the single-stranded 5′ uncapped triphosphate or biphosphate modified RNA oligonucleotide comprises a nucleic acid sequence that is 100% identical to the nucleic acid sequence of SEQ ID NO: 13. In some embodiments, the single-stranded 5′ uncapped triphosphate or biphosphate modified RNA oligonucleotide comprises a nucleic acid sequence that is identical to the nucleic acid sequence of SEQ ID NO: 13. In some embodiments, the single-stranded 5′ uncapped triphosphate or biphosphate modified RNA oligonucleotide consists of a nucleic acid sequence that is identical to a nucleic acid sequence selected from SEQ ID NO: 13.

Antisense Oligonucleotides (ASOs)

Antisense oligonucleotide (ASOs) are small-sized single-stranded nucleic acids (typically at least 8 or 10 nts and up to about 30 nts for miRNA, or much longer for full-length mRNA antisense RNAs) that bind to their target RNA sequence inside the cells mediating gene silencing. The ASO-based strategy targets the disease source at the RNA level rather than targeting downstream processes involving in proteins. Proteins are produced by decoding information stored in messenger RNA (mRNA). Aberrant protein production, which is associated with numerous devastating diseases and disorders, can be regulated by targeting mRNA through the action of non-coding RNAs (ncRNAs). Among ncRNAs, microRNA (miRNA), transfer RNA-derived small RNA, pseudogenes, PIWI-interacting RNA, long ncRNAs (lncRNAs), and circular RNAs have been identified as regulators of biological functions through modulation of gene expression. Hence, the antisense strategy comprising of targeting pre-mRNA, mRNA, or ncRNAs including miRNA can alter the production of disease-causing proteins for therapeutic interventions.

Unlike small molecule-based protein targeting, antisense drugs exhibit their effect by Watson-Crick base pairing rules with target RNA sequence. This principle of Watson-Crick molecular recognition provides the antisense field more flexibility in RNA-based drug design and expedites its development. However, during ASO design, necessary modifications to optimize binding affinity, improve nuclease resistance, and in vivo delivery can be considered. There have been several generations of designs with attempts to develop AMOs with high binding affinity, high specificity, and expanded functionality (Ochoa S, Milam V T. Modified Nucleic Acids: Expanding the Capabilities of Functional Oligonucleotides. Molecules. 2020; 25(20):4659).

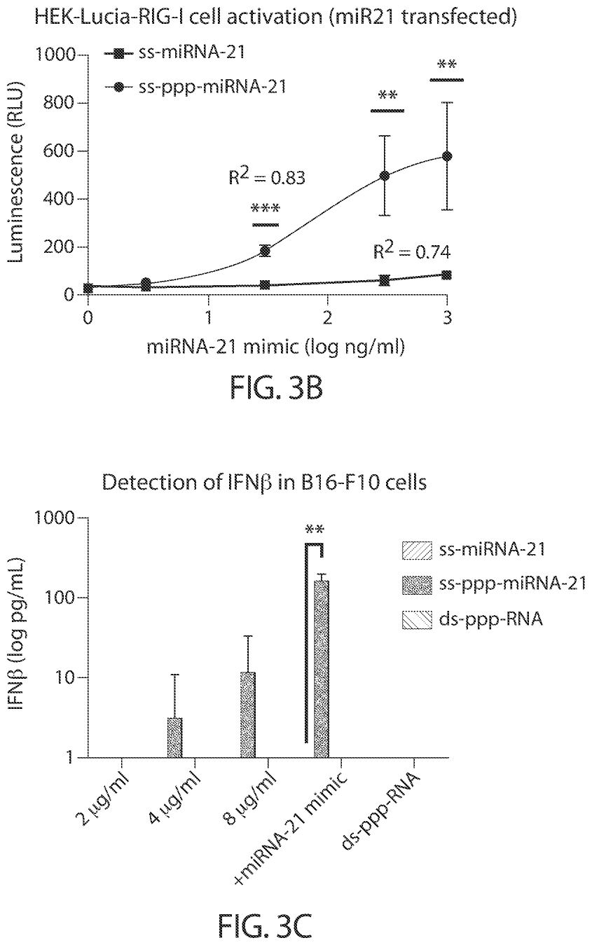

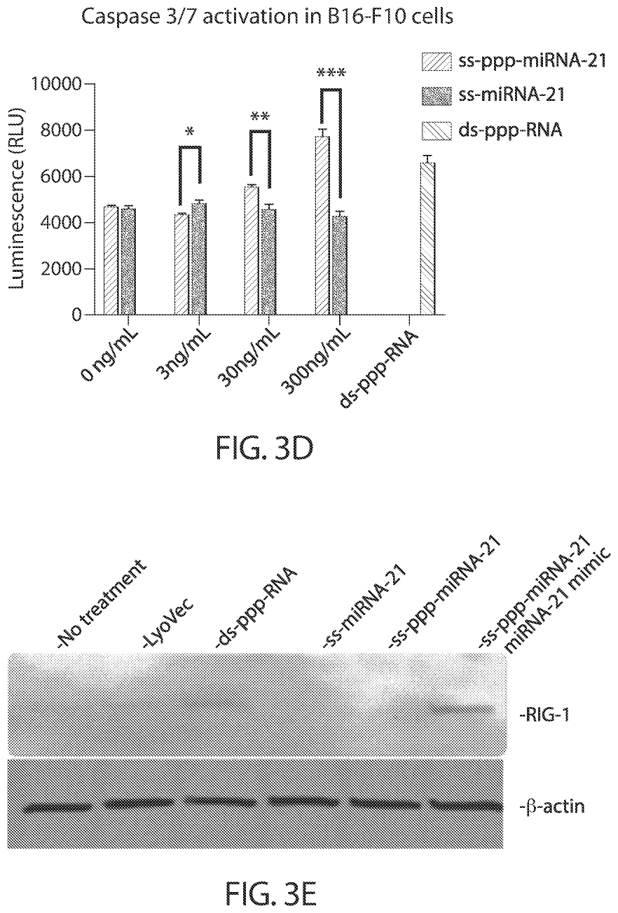

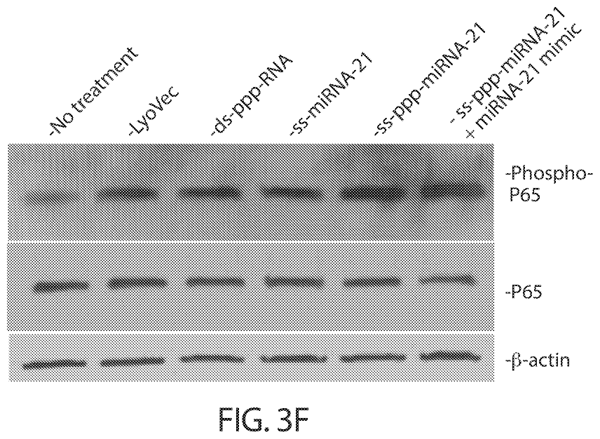

An RNA nucleotide can be chemically modified at the backbone, nucleobase, ribose sugar and 2′-ribose substitutions. see FIG. 3 of Roberts, T. C., Langer, R. & Wood, M. J. A. Advances in oligonucleotide drug delivery. Nat Rev Drug Discov 19, 673-694 (2020).

In some ASOs (often referred to as first-generation), the phosphate backbone linking the nucleotides is modified. One of the non-bridging oxygen atoms in the phosphodiester bond is replaced by a sulfur, methyl or amine group, generating phosphorothioates (PS), methyl-phosphonates, and phosphoramidates, respectively. These modifications are not equivalent, and each has its own specific features. PS oligonucleotides are highly representative of this first generation, being the most widely used. These chemical modifications improve stability by increasing the resistance of ASOs to nucleases, a constant goal being to expand the ASO half-life. PS modifications transform the half-life from minutes to days. Importantly, these modifications activate RNAse H. RNAse H is a ubiquitously expressed enzyme that cleaves the RNA strand in a DNA-RNA duplex. RNAse H can, therefore, degrade the target mRNA within the ASO/mRNA complexes, limiting the synthesis of the encoded protein. Unfortunately, the biologically active modified ASOs (PS) are highly toxic due, in particular, to their non-specific binding to proteins. This led researchers to develop new generations of ASOs that were both less toxic and more specific.

Another class of ASOs (sometimes referred to as second generation ASOs) is characterized by alkyl modifications at the 2′ position of the ribose. The introduction of an oxygenated group leads to the formation of 2′-O-methyl (2′-OME) and 2′-O-methoxyethyl (2′-MOE) nucleotides. These ASOs are less toxic than PS and have a slightly higher affinity for their target. However, these modifications are incompatible with the recruitment of and cleavage by RNAse H. The antisense effect of this type of ASO is probably due to steric blockade of translation. Such modifications are of potential interest if the target RNA must not be degraded.

A further class of ASOs (sometimes referred to as third generation ASOs) is more heterogeneous, including a large number of modifications aiming to improve binding-affinity, resistance to nucleases, and pharmacokinetic profile. The most common modifications include locked nucleic acids (LNAs), corresponding to a methylene bridge connecting the 2′-oxygen and 4′-carbon of the ribose; phosphorodiamidate morpholino oligomers (PMOs), in which the ribose is replaced by a morpholine moiety and the phosphodiester bond by a phosphorodiamidate bond; and peptide nucleic acids (PNAs), in which the ribose-phosphate backbone is replaced by a polyamide backbone consisting of repeats of N-(2-aminothyl) glycine units, to which the bases are linked (Papargyri N, Pontoppidan M, Andersen M R, Koch T, Hagedorn P H. Chemical Diversity of Locked Nucleic Acid-Modified Antisense Oligonucleotides Allows Optimization of Pharmaceutical Properties. Mol Ther Nucleic Acids. 2020; 19:706-717). These last two structures are uncharged and bind to plasma proteins with a lower affinity than charged ASOs, which increases their distribution and elimination in urine. The fraction eliminated has been shown to correspond to approximately 10-30% of the amount administered, contributing to tissue accumulation. These modifications confer high stability but do not elicit RNAse H recruitment. This third generation of ASO forms a stable hybrid with its target mRNA, thereby interfering with its processing or translation.

The conformational constraint of the LNA modification imposed by the connecting bridge and that of its methylated analog (known as “constrained ethyl”: cET) have created new opportunities in chemical therapeutics. Tricyclo-DNA (tcDNA) belongs to this class of conformationally constrained DNA analogs with enhanced binding properties. They do not elicit RNAse-H activity but show increased stability and improved cellular uptake, giving them substantial therapeutic advantages over those of ASOs.

As underscored previously, ASOs carrying most second- and third-generation chemical modifications do not elicit RNAse H activity. RNAse H activity can however be restored by inserting a stretch of unmodified or PS-DNA cleavage-sensitive sequence between a pair of non-RNAse H-sensitive sequences at the ends of the ASO. The resulting structure is known as a “gapmer.” (see, e.g., Quemener, Wiley Interdiscip Rev RNA. 2020:11(5):e1594).

Overall, such diversity of chemical modifications, together with the structure of the ASO, offers considerable flexibility for the adaptation of the therapeutic approach according to the chosen target and mechanism of action. Recent United States Food and Drug Administration (FDA) approval of several nucleic acid-based drugs has further spurred interest in the antisense research. Presently, numerous antisense drug candidates are in clinical trials to treat cardiovascular, metabolic, endocrine, neurological, neuromuscular, inflammatory, and infectious diseases.

In some embodiments, the antisense oligos include one or more modifications, e.g., in a base, 2′position, backbone/phosphate, or ribose, as known in the art or described herein, so long as the modifications don't interfere with the interaction with the RIG-I helicase.

Anti-miRNA Oligonucleotides (AMOs)

As noted above, microRNAs tightly regulate gene expression, thereby controlling many physiological functions. Because they are important regulators, they are also associated with disease. Inhibition of their activity may, therefore, be an effective therapeutic strategy. AMOs are ASOs with sequence complementary to the endogenous miRNA targeted, forming stable, high-affinity bonds. Like ASOs, they can be synthesized with various chemical characteristics, as described above. These synthetically designed molecules are used to neutralize microRNA (miRNA) function in cells for desired responses through a steric blocking mechanism as well as hybridization to miRNA. In particular, it is essential that the AMO binds with high affinity to the miRNA ‘seed region’, which spans bases 2-8 from the 5′-end of the miRNA.

### 5. Methods of Treatment

In part, the present disclosure relates to methods for treating cancer comprising administering a therapeutically effective amount of a single-stranded 5′ uncapped triphosphate or biphosphate modified RNA oligonucleotide, wherein said oligonucleotide is complementary to an endogenous tumor-specific RNA (tsRNA), which is highly expressed in tumor cells in comparison to non-tumor cells. In some embodiments, the disclosure contemplates methods for selectively activating RIG-I in tumor cells comprising administering a therapeutically effective amount single-stranded 5′ uncapped triphosphate or biphosphate modified RNA oligonucleotide, wherein said single-stranded 5′ uncapped triphosphate or biphosphate modified RNA oligonucleotide comprises a sequence which is complementary to an endogenous tumor-specific RNA (tsRNA), wherein the RIG-I is selectively activated in tumor cells highly expressing the tumor-specific RNA. In some embodiments, the RNA is mRNA. In some embodiments, the RNA is miRNA. In some embodiments, the miRNA is selected from the group consisting of SEQ ID NOs: 1-13.

The terms “treatment”, “treating”, “alleviating” and the like are used herein to generally mean obtaining a desired pharmacologic and/or physiologic effect, and may also be used to refer to improving, alleviating, and/or decreasing the severity of one or more clinical complication of a condition being treated (e.g., cancer). The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure (e.g., cancer) is achieved by administering one or more 5′pp or 5′ppp ss RNA oligonucleotides of the present disclosure in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

In certain aspects, the disclosure contemplates the use of one or more 5′pp or 5′ppp ss RNA oligonucleotides, in combination with one or more additional active agents or other supportive therapy for treating or preventing a disease or condition (e.g., cancer). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more 5′pp or 5′ppp ss RNA oligonucleotides of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the 5′pp or 5′ppp ss RNA oligonucleotide of the present disclosure with the additional active agent or therapy and/or the desired effect.

The methods described herein include methods for treating cancer comprising administering a single-stranded 5′ uncapped triphosphate or biphosphate modified RNA oligonucleotide, wherein said oligonucleotide is complementary to a miRNA or mRNA which is highly expressed in tumor cells in comparison to non-tumor cells. Without wishing to be bound by theory, the single-stranded 5′ uncapped triphosphate or biphosphate modified RNA oligonucleotide forms a duplex with the tumor specific RNA thereby eliciting a tumor specific immune response via the RIG-I signaling pathway. As such, provided herein is a method of treating cancer by combining RIG-I mediated immune activation against tumor cells while, optionally, inhibiting a miRNA or mRNA (e.g., use of a 5′pp or 5′ppp ss RNA oligonucleotide which is complementary to endogenous miR21). In some embodiments, the endogenously expressed mRNA or miRNA is oncogenic. In some embodiments, the endogenously expressed mRNA or miRNA is tumor-specific. As used herein, “tumor-specific RNA” refers to RNA (e.g., miRNA or mRNA) which is highly expressed in tumor cells as compared to non-tumor cells.

In vivo, miRNAs often exert regulatory functions within RNA-induced silencing complexes (RISCs). The core subunit of RISC is a miRNA bound to AGO2 (a member of the Argonaute family of proteins). The miRNA within the RISC complex comprises double-stranded miRNA, with one RNA strand being the miRNA-guide which guides the complex to the target mRNA, and the other RNA strand being the passenger strand, which is removed from the complex and degraded. AGO2 uses the miRNA-guide to identify a complementary target transcript for repression.

In some embodiments, the single-stranded 5′ uncapped triphosphate or biphosphate modified RNA oligonucleotide forms a duplex with the endogenous tumor-specific RNA. In some embodiments, the endogenous tumor-specific RNA is selected from miRNA or mRNA. In some embodiments, the miRNA or mRNA is oncogenic. In some embodiments, the single-stranded 5′ uncapped triphosphate or biphosphate modified RNA oligonucleotide forms a duplex with the miRNA. In some embodiments, the duplex is not cleaved by AGO2. In some embodiments, the duplex is released by AGO2. In some embodiments, the duplex comprises between 0-5 mismatched base pairs.

In some embodiments, the duplex activates RIG-1. In some embodiments, the RIG-I activation is at least 5%, 10%, 15% or 20% greater than activation by a corresponding unmodified monophosphate RNA oligonucleotide. In some embodiments, the RIG-I activation elicits a tumor specific immune response. In some embodiments, the single-stranded 5′ uncapped triphosphate or biphosphate modified RNA oligonucleotide competes with endogenous mRNA to bind the miRNA.

The methods disclosed herein include the treatment of disorders associated with abnormal apoptotic or differentiative processes, e.g., cellular proliferative disorders or cellular differentiative disorders, e.g., cancer, including both solid tumors and hematopoietic cancers. In certain embodiments, the methods are directed to a dual method of treatment comprising the combination of tumor-specific immune activation and inhibition of miRNA or mRNA. The methods can also be used to reduce the risk of developing disorders associated with abnormal apoptotic or differentiative processes, by triggering an immune response that targets developing cancer cells. In some embodiments, the disorder is a solid tumor, e.g., breast, prostate, pancreatic, brain, hepatic, lung, kidney, skin, or colon cancer. Generally, the methods include administering a therapeutically effective amount of a treatment as described herein, to a subject who is in need of, or who has been determined to be in need of, such treatment. In some embodiments, the methods include administering a therapeutically effective amount of a treatment comprising a 5′pp or 5′ppp ss RNA oligonucleotide (e.g., RNA oligonucleotide as used herein), e.g., linked to a nanoparticle. In some embodiments, the nanoparticle is a magnetic nanoparticle.

As used in this context, to “treat” means to ameliorate at least one symptom of the disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes (e.g., cancer) will result in a reduction in tumor size or decreased growth rate, a reduction in risk or frequency of reoccurrence, a delay in reoccurrence, a reduction in metastasis, increased survival, and/or decreased morbidity and mortality, inter alia.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting bladder, bone, lung, kidney, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Other types of cancers include, but are not limited to biliary tract cancer, brain cancer, breast cancer, cervical cancer, choriocarcinoma, colon cancer, endometrial cancer, esophageal cancer, gastric cancer, glioblastoma, intraepithelial neoplasm, leukemia, lymphoma, liver cancer, lung cancer, melanoma, myelomas, neuroblastoma, oral cancer, ovarian cancer, pancreatic cancer, prostate cancer, rectal cancer, sarcoma, skin cancer, stomach cancer, testicular cancer, thyroid cancer, uterine cancer, and renal cancer. In certain embodiments, the cancer is selected from hairy cell leukemia, chronic myelogenous leukemia, cutaneous T-cell leukemia, chronic myeloid leukemia, non-Hodgkin's lymphoma, multiple myeloma, follicular lymphoma, malignant melanoma, squamous cell carcinoma, renal cell carcinoma, prostate carcinoma, bladder cell carcinoma, breast carcinoma, ovarian carcinoma, non-small cell lung cancer, small cell lung cancer, hepatocellular carcinoma, basaliom, colon carcinoma, cervical dysplasia, and Kaposi's sarcoma (AIDS-related and non-AIDS related).

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments of any of the methods described herein, the 5′pp or 5′ppp ss RNA oligonucleotide (e.g., RNA oligonucleotide as used herein) is administered to a subject that has been diagnosed as having a cancer (e.g., having a primary cancer or a metastatic cancer). In some embodiments, the subject has breast cancer (e.g., a metastatic breast cancer). In some non-limiting embodiments, the subject is a man or a woman, an adult, an adolescent, or a child. In some embodiments, the subject has one or more symptoms of a cancer or metastatic cancer (e.g., a metastatic cancer in a lymph node). In some embodiments, the subject has severe or an advanced stage of cancer (e.g., a primary or metastatic cancer). In some embodiments, the subject has a metastatic tumor present in at least one lymph node. In some embodiments, the subject has undergone lymphectomy and/or mastectomy.

RIG-I Receptor Activated Immune Response

As described previously, RIG-I is a cytosolic nucleic acid sensing Pattern Recognition Receptor (PRR) of the innate immune system. It is essential for recognizing RNA structures (like viruses) with a 5′ triphosphate signature. RIG-I activation can be programmed as an immune response against cancer. Importantly, tumor cell death through RIG-I has been shown to build immunological memory, meaning that once the body's immune system has been activated, the body becomes immune, and tumors are rejected as “foreign.”

In some embodiments, the disclosure contemplates methods for selectively activating RIG-I in tumor cells comprising administering a therapeutically effective amount single-stranded 5′ uncapped triphosphate or biphosphate modified RNA oligonucleotide, wherein said single-stranded 5′ uncapped triphosphate or biphosphate modified RNA oligonucleotide comprises a sequence which is complementary to an endogenous tumor-specific RNA (tsRNA), wherein the RIG-I is selectively activated in tumor cells highly expressing the tumor-specific RNA. Without wishing to be bound by theory, the immune system is selectively activated in cancer cells when the single-stranded 5′ uncapped triphosphate or biphosphate modified RNA oligonucleotide forms a duplex with the tumor specific RNA thereby eliciting a tumor specific immune response via the RIG-I signaling pathway. In some embodiments, administration of the 5′pp or 5′ppp ss RNA oligonucleotide induces an anti-viral response, in particular, a type I IFN response. In some embodiments, the type I IFN response is an IFN-α response. In some embodiments, the RIG-I activation elicits a tumor specific immune response (e.g., a response against a tumor cell which highly expresses the tumor specific RNA). In some embodiments, the tumor specific immune response comprises release of type I IFNs, DAMPs (danger-associated molecular pattern), and/or tumor antigens. In some embodiments, the method induces immunological memory against said tumor cells.

In some embodiments, the administration of the 5′pp or 5′ppp ss RNA oligonucleotide induces apoptosis of a tumor cell. In some embodiments, the administration of the 5′pp or 5′ppp ss RNA oligonucleotide (a) induces an anti-viral response, in particular, a type I IFN response, and b) downregulates a tumor-specific RNA (e.g., miRNA21) in a vertebrate animal, in particular, a mammal. The present application further provides the use of at least one 5′pp or 5′ppp ss RNA oligonucleotide for the preparation of a pharmaceutical composition for inducing apoptosis of a tumor cell in a vertebrate animal, in particular, a mammal.

Described herein are methods and/or compositions for eliciting a tumor specific immune response through the administration of 5′pp or 5′ppp ss RNA oligonucleotides thereby activating the body's immune system to effect the desired treatment response (e.g., treating and/or creating an anti-tumor immunological memory in an animal). Without wishing to be bound by theory, as shown in FIG. 1, the RIG-I pathway is selectively activated in cancer cells by in situ generation of 5′ppp-dsRNA following introduction of 5′ppp ss RNA oligonucleotide complementary to a miRNA or mRNA expressed specifically in these cells: the same or similar is expected from 5′pp-ssRNA. Consequently, the antitumor immunity potential of the tumor microenvironment (TME) can be uncovered via the activation of RIG-I signaling pathway, in conjunction with concurrent activation of certain tumor suppressor gene(s), by simply using a single-stranded RNA.

### 6. Pharmaceutical Compositions & Modes of Administration

In any of the methods described herein, the 5′pp or 5′ppp ss RNA oligonucleotide (e.g., RNA oligonucleotide as used herein) can be administered by a health care professional (e.g., a physician, a physician's assistant, a nurse, or a laboratory or clinic worker), the subject (i.e., self-administration), or a friend or family member of the subject. The administering can be performed in a clinical setting (e.g., at a clinic or a hospital), in an assisted living facility, or at a pharmacy.

In some embodiments of any of the methods described herein, the subject is administered at least one (e.g., at least 2, 3, 4, 5, 6, 7, 8, 9, 10, 15, 20, 25, or 30) dose of a composition containing at least one (e.g., one, two, three, or four) of any of the 5′pp or 5′ppp ss RNA oligonucleotides (e.g., RNA oligonucleotide as used herein) described herein. In any of the methods described herein, the at least one magnetic particle or pharmaceutical composition (e.g., any of the magnetic particles or pharmaceutical compositions described herein) can be administered intravenously, intraarterially, subcutaneously, intraperitoneally, or intramuscularly to the subject. In some embodiments, the at least one magnetic particle or pharmaceutical composition is directly administered (injected) into a lymph node in a subject.

In some embodiments of any of the methods described herein, the subject is administered at least one (e.g., at least 2, 3, 4, 5, 6, 7, 8, 9, 10, 15, 20, 25, or 30) dose of a composition containing at least one (e.g., one, two, three, or four) of any of the 5′pp or 5′ppp ss RNA oligonucleotides (e.g., RNA oligonucleotide as used herein) described herein. In some embodiments, the 5′pp or 5′ppp ss RNA oligonucleotides of the disclosure are administered at a dosing range of 0.2 mg/kg to 200 mg/kg. In some embodiments, the 5′pp or 5′ppp ss RNA oligonucleotides of the disclosure are administered at a dosing range of 0.3 mg/kg to 200 mg/kg. In some embodiments, the 5′pp or 5′ppp ss RNA oligonucleotides of the disclosure are administered at a dosing range of 0.4 mg/kg to 200 mg/kg. In some embodiments, the 5′pp or 5′ppp ss RNA oligonucleotides of the disclosure are administered at a dosing range of 0.5 mg/kg to 200 mg/kg. In some embodiments, the 5′pp or 5′ppp ss RNA oligonucleotides of the disclosure are administered at a dosing range of 0.6 mg/kg to 200 mg/kg. In some embodiments, the 5′pp or 5′ppp ss RNA oligonucleotides of the disclosure are administered at a dosing range of 0.7 mg/kg to 200 mg/kg. In some embodiments, the 5′pp or 5′ppp ss RNA oligonucleotides of the disclosure are administered at a dosing range of 0.8 mg/kg to 200 mg/kg. In some embodiments, the 5′pp or 5′ppp ss RNA oligonucleotides of the disclosure are administered at a dosing range of 0.9 mg/kg to 200 mg/kg. In some embodiments, the 5′pp or 5′ppp ss RNA oligonucleotides of the disclosure are administered at a dosing range of 1 mg/kg to 200 mg/kg. In some embodiments, the 5′pp or 5′ppp ss RNA oligonucleotides of the disclosure are administered at a dosing range of 2 mg/kg to 200 mg/kg. In some embodiments, the 5′pp or 5′ppp ss RNA oligonucleotides of the disclosure are administered at a dosing range of 3 mg/kg to 200 mg/kg. In some embodiments, the 5′pp or 5′ppp ss RNA oligonucleotides of the disclosure are administered at a dosing range of 4 mg/kg to 200 mg/kg. In some embodiments, the 5′pp or 5′ppp ss RNA oligonucleotides of the disclosure are administered at a dosing range of 5 mg/kg to 200 mg/kg. In some embodiments, the 5′pp or 5′ppp ss RNA oligonucleotides of the disclosure are administered at a dosing range of 6 mg/kg to 200 mg/kg. In some embodiments, the 5′pp or 5′ppp ss RNA oligonucleotides of the disclosure are administered at a dosing range of 7 mg/kg to 200 mg/kg. In some embodiments, the 5′pp or 5′ppp ss RNA oligonucleotides of the disclosure are administered at a dosing range of 8 mg/kg to 200 mg/kg. In some embodiments, the 5′pp or 5′ppp ss RNA oligonucleotides of the disclosure are administered at a dosing range of 9 mg/kg to 200 mg/kg. In some embodiments, the 5′pp or 5′ppp ss RNA oligonucleotides of the disclosure are administered at a dosing range of 10 mg/kg to 200 mg/kg. In some embodiments, the 5′pp or 5′ppp ss RNA oligonucleotides of the disclosure are administered at a dosing range of 20 mg/kg to 200 mg/kg. In some embodiments, the 5′pp or 5′ppp ss RNA oligonucleotides of the disclosure are administered at a dosing range of 30 mg/kg to 200 mg/kg. In some embodiments, the 5′pp or 5′ppp ss RNA oligonucleotides of the disclosure are administered at a dosing range of 40 mg/kg to 200 mg/kg. In some embodiments, the 5′pp or 5′ppp ss RNA oligonucleotides of the disclosure are administered at a dosing range of 50 mg/kg to 200 mg/kg. In some embodiments, the 5′pp or 5′ppp ss RNA oligonucleotides of the disclosure are administered at a dosing range of 60 mg/kg to 200 mg/kg. In some embodiments, the 5′pp or 5′ppp ss RNA oligonucleotides of the disclosure are administered at a dosing range of 70 mg/kg to 200 mg/kg. In some embodiments, the 5′pp or 5′ppp ss RNA oligonucleotides of the disclosure are administered at a dosing range of 80 mg/kg to 200 mg/kg. In some embodiments, the 5′pp or 5′ppp ss RNA oligonucleotides of the disclosure are administered at a dosing range of 90 mg/kg to 200 mg/kg. In some embodiments, the 5′pp or 5′ppp ss RNA oligonucleotides of the disclosure are administered at a dosing range of 100 mg/kg to 200 mg/kg. In some embodiments, the 5′pp or 5′ppp ss RNA oligonucleotides of the disclosure are administered at a dosing range of 110 mg/kg to 200 mg/kg. In some embodiments, the 5′pp or 5′ppp ss RNA oligonucleotides of the disclosure are administered at a dosing range of 120 mg/kg to 200 mg/kg. In some embodiments, the 5′pp or 5′ppp ss RNA oligonucleotides of the disclosure are administered at a dosing range of 130 mg/kg to 200 mg/kg. In some embodiments, the 5′pp or 5′ppp ss RNA oligonucleotides of the disclosure are administered at a dosing range of 140 mg/kg to 200 mg/kg. In some embodiments, the 5′pp or 5′ppp ss RNA oligonucleotides of the disclosure are administered at a dosing range of 150 mg/kg to 200 mg/kg. In some embodiments, the 5′pp or 5′ppp ss RNA oligonucleotides of the disclosure are administered at a dosing range of 160 mg/kg to 200 mg/kg. In some embodiments, the 5′pp or 5′ppp ss RNA oligonucleotides of the disclosure are administered at a dosing range of 170 mg/kg to 200 mg/kg. In some embodiments, the 5′pp or 5′ppp ss RNA oligonucleotides of the disclosure are administered at a dosing range of 180 mg/kg to 200 mg/kg. In some embodiments, the 5′pp or 5′ppp ss RNA oligonucleotides of the disclosure are administered at a dosing range of 190 mg/kg to 200 mg/kg.

In some embodiments, the 5′pp or 5′ppp ss RNA oligonucleotides of the disclosure are administered at a dose of at least 0.2 mg/kg. In some embodiments, the 5′pp or 5′ppp ss RNA oligonucleotides of the disclosure are administered at a dose of at least 0.3 mg/kg. In some embodiments, the 5′pp or 5′ppp ss RNA oligonucleotides of the disclosure are administered at a dose of at least 0.4 mg/kg. In some embodiments, the 5′pp or 5′ppp ss RNA oligonucleotides of the disclosure are administered at a dose of at least 0.5 mg/kg. In some embodiments, the 5′pp or 5′ppp ss RNA oligonucleotides of the disclosure are administered at a dose of at least 0.6 mg/kg. In some embodiments, the 5′pp or 5′ppp ss RNA oligonucleotides of the disclosure are administered at a dose of at least 0.7 mg/kg. In some embodiments, the 5′pp or 5′ppp ss RNA oligonucleotides of the disclosure are administered at a dose of at least 0.8 mg/kg. In some embodiments, the 5′pp or 5′ppp ss RNA oligonucleotides of the disclosure are administered at a dose of at least 0.9 mg/kg. In some embodiments, the 5′pp or 5′ppp ss RNA oligonucleotides of the disclosure are administered at a dose of at least 1 mg/kg. In some embodiments, the 5′pp or 5′ppp ss RNA oligonucleotides of the disclosure are administered at a dose of at least 2 mg/kg. In some embodiments, the 5′pp or 5′ppp ss RNA oligonucleotides of the disclosure are administered at a dose of at least 3 mg/kg. In some embodiments, the 5′pp or 5′ppp ss RNA oligonucleotides of the disclosure are administered at a dose of at least 4 mg/kg. In some embodiments, the 5′pp or 5′ppp ss RNA oligonucleotides of the disclosure are administered at a dose of at least 5 mg/kg. In some embodiments, the 5′pp or 5′ppp ss RNA oligonucleotides of the disclosure are administered at a dose of at least 6 mg/kg. In some embodiments, the 5′pp or 5′ppp ss RNA oligonucleotides of the disclosure are administered at a dose of at least 7 mg/kg. In some embodiments, the 5′pp or 5′ppp ss RNA oligonucleotides of the disclosure are administered at a dose of at least 8 mg/kg. In some embodiments, the 5′pp or 5′ppp ss RNA oligonucleotides of the disclosure are administered at a dose of at least 9 mg/kg. In some embodiments, the 5′pp or 5′ppp ss RNA oligonucleotides of the disclosure are administered at a dose of at least 10 mg/kg. In some embodiments, the 5′pp or 5′ppp ss RNA oligonucleotides of the disclosure are administered at a dose of at least 20 mg/kg. In some embodiments, the 5′pp or 5′ppp ss RNA oligonucleotides of the disclosure are administered at a dose of at least 30 mg/kg. In some embodiments, the 5′pp or 5′ppp ss RNA oligonucleotides of the disclosure are administered at a dose of at least 40 mg/kg. In some embodiments, the 5′pp or 5′ppp ss RNA oligonucleotides of the disclosure are administered at a dose of at least 50 mg/kg. In some embodiments, the 5′pp or 5′ppp ss RNA oligonucleotides of the disclosure are administered at a dose of at least 60 mg/kg. In some embodiments, the 5′pp or 5′ppp ss RNA oligonucleotides of the disclosure are administered at a dose of at least 70 mg/kg. In some embodiments, the 5′pp or 5′ppp ss RNA oligonucleotides of the disclosure are administered at a dose of at least 80 mg/kg. In some embodiments, the 5′pp or 5′ppp ss RNA oligonucleotides of the disclosure are administered at a dose of at least 90 mg/kg. In some embodiments, the 5′pp or 5′ppp ss RNA oligonucleotides of the disclosure are administered at a dose of at least 100 mg/kg. In some embodiments, the 5′pp or 5′ppp ss RNA oligonucleotides of the disclosure are administered at a dose of at least 110 mg/kg. In some embodiments, the 5′pp or 5′ppp ss RNA oligonucleotides of the disclosure are administered at a dose of at least 120 mg/kg. In some embodiments, the 5′pp or 5′ppp ss RNA oligonucleotides of the disclosure are administered at a dose of at least 130 mg/kg. In some embodiments, the 5′pp or 5′ppp ss RNA oligonucleotides of the disclosure are administered at a dose of at least 140 mg/kg. In some embodiments, the 5′pp or 5′ppp ss RNA oligonucleotides of the disclosure are administered at a dose of at least 150 mg/kg. In some embodiments, the 5′pp or 5′ppp ss RNA oligonucleotides of the disclosure are administered at a dose of at least 160 mg/kg. In some embodiments, the 5′pp or 5′ppp ss RNA oligonucleotides of the disclosure are administered at a dose of at least 170 mg/kg. In some embodiments, the 5′pp or 5′ppp ss RNA oligonucleotides of the disclosure are administered at a dose of at least 180 mg/kg. In some embodiments, the 5′pp or 5′ppp ss RNA oligonucleotides of the disclosure are administered at a dose of at least 190 mg/kg. In some embodiments, the 5′pp or 5′ppp ss RNA oligonucleotides of the disclosure are administered at a dose of at least 200 mg/kg.

In certain embodiments, the 5′pp or 5′ppp ss RNA oligonucleotides of the disclosure are administered once a day. In certain embodiments, the 5′pp or 5′ppp ss RNA oligonucleotides of the disclosure are administered twice a day. In certain embodiments, the 5′pp or 5′ppp ss RNA oligonucleotides of the disclosure are administered once a week. In certain embodiments, the 5′pp or 5′ppp ss RNA oligonucleotides of the disclosure are administered twice a week. In certain embodiments, the 5′pp or 5′ppp ss RNA oligonucleotides of the disclosure are administered three times a week. In certain embodiments, the 5′pp or 5′ppp ss RNA oligonucleotides of the disclosure are administered every two weeks. In certain embodiments, the 5′pp or 5′ppp ss RNA oligonucleotides of the disclosure are administered every three weeks. In certain embodiments, the 5′pp or 5′ppp ss RNA oligonucleotides of the disclosure are administered every four weeks. In certain embodiments, the 5′pp or 5′ppp ss RNA oligonucleotides of the disclosure are administered every month.

In some embodiments of any of the methods described herein, the subject is administered at least one (e.g., at least 2, 3, 4, 5, 6, 7, 8, 9, 10, 15, 20, 25, or 30) dose of a composition containing at least one (e.g., one, two, three, or four) of any of the 5′pp or 5′ppp ss RNA oligonucleotides (e.g., RNA oligonucleotide as used herein) described herein. In certain embodiments, the modified RNA oligonucleotide comprises up to 40 different modified RNA oligonucleotides. In certain embodiments, the modified RNA oligonucleotide comprises up to 39 different modified RNA oligonucleotides. In certain embodiments, the modified RNA oligonucleotide comprises up to 38 different modified RNA oligonucleotides. In certain embodiments, the modified RNA oligonucleotide comprises up to 37 different modified RNA oligonucleotides. In certain embodiments, the modified RNA oligonucleotide comprises up to 36 different modified RNA oligonucleotides. In certain embodiments, the modified RNA oligonucleotide comprises up to 35 different modified RNA oligonucleotides. In certain embodiments, the modified RNA oligonucleotide comprises up to 34 different modified RNA oligonucleotides. In certain embodiments, the modified RNA oligonucleotide comprises up to 33 different modified RNA oligonucleotides. In certain embodiments, the modified RNA oligonucleotide comprises up to 32 different modified RNA oligonucleotides. In certain embodiments, the modified RNA oligonucleotide comprises up to 31 different modified RNA oligonucleotides. In certain embodiments, the modified RNA oligonucleotide comprises up to 30 different modified RNA oligonucleotides. In certain embodiments, the modified RNA oligonucleotide comprises up to 29 different modified RNA oligonucleotides. In certain embodiments, the modified RNA oligonucleotide comprises up to 28 different modified RNA oligonucleotides. In certain embodiments, the modified RNA oligonucleotide comprises up to 27 different modified RNA oligonucleotides. In certain embodiments, the modified RNA oligonucleotide comprises up to 26 different modified RNA oligonucleotides. In certain embodiments, the modified RNA oligonucleotide comprises up to 25 different modified RNA oligonucleotides. In certain embodiments, the modified RNA oligonucleotide comprises up to 24 different modified RNA oligonucleotides. In certain embodiments, the modified RNA oligonucleotide comprises up to 23 different modified RNA oligonucleotides. In certain embodiments, the modified RNA oligonucleotide comprises up to 22 different modified RNA oligonucleotides. In certain embodiments, the modified RNA oligonucleotide comprises up to 21 different modified RNA oligonucleotides. In certain embodiments, the modified RNA oligonucleotide comprises up to 20 different modified RNA oligonucleotides. In certain embodiments, the modified RNA oligonucleotide comprises up to 19 different modified RNA oligonucleotides. In certain embodiments, the modified RNA oligonucleotide comprises up to 18 different modified RNA oligonucleotides. In certain embodiments, the modified RNA oligonucleotide comprises up to 17 different modified RNA oligonucleotides. In certain embodiments, the modified RNA oligonucleotide comprises up to 16 different modified RNA oligonucleotides. In certain embodiments, the modified RNA oligonucleotide comprises up to 15 different modified RNA oligonucleotides. In certain embodiments, the modified RNA oligonucleotide comprises up to 14 different modified RNA oligonucleotides. In certain embodiments, the modified RNA oligonucleotide comprises up to 13 different modified RNA oligonucleotides. In certain embodiments, the modified RNA oligonucleotide comprises up to 12 different modified RNA oligonucleotides. In certain embodiments, the modified RNA oligonucleotide comprises up to 11 different modified RNA oligonucleotides. In certain embodiments, the modified RNA oligonucleotide comprises up to 10 different modified RNA oligonucleotides. In certain embodiments, the modified RNA oligonucleotide comprises up to 9 different modified RNA oligonucleotides. In certain embodiments, the modified RNA oligonucleotide comprises up to 8 different modified RNA oligonucleotides. In certain embodiments, the modified RNA oligonucleotide comprises up to 7 different modified RNA oligonucleotides. In certain embodiments, the modified RNA oligonucleotide comprises up to 6 different modified RNA oligonucleotides. In certain embodiments, the modified RNA oligonucleotide comprises up to 5 different modified RNA oligonucleotides. In certain embodiments, the modified RNA oligonucleotide comprises up to 4 different modified RNA oligonucleotides. In certain embodiments, the modified RNA oligonucleotide comprises up to 3 different modified RNA oligonucleotides. In certain embodiments, the modified RNA oligonucleotide comprises up to 2 different modified RNA oligonucleotides. In certain embodiments, the modified RNA oligonucleotide comprises a modified RNA oligonucleotide.

In some embodiments of any of the methods described herein, the subject is administered at least one (e.g., at least 2, 3, 4, 5, 6, 7, 8, 9, 10, 15, 20, 25, or 30) dose of a composition containing at least one (e.g., one, two, three, or four) of any of the 5′pp or 5′ppp ss RNA oligonucleotides (e.g., RNA oligonucleotide as used herein) described herein. In any of the methods described herein, the at least one magnetic particle or pharmaceutical composition (e.g., any of the magnetic particles or pharmaceutical compositions described herein) can be administered intravenously, intraarterially, subcutaneously, intraperitoneally, or intramuscularly to the subject. In some embodiments, the at least one magnetic particle or pharmaceutical composition is directly administered (injected) into a lymph node in a subject. In some embodiments, the magnetic nanoparticle comprises between 1 to up to 40 different modified RNA oligonucleotides. In some embodiments, the magnetic nanoparticle comprises between 2 to up to 40 different modified RNA oligonucleotides. In some embodiments, the magnetic nanoparticle comprises between 3 to up to 40 different modified RNA oligonucleotides. In some embodiments, the magnetic nanoparticle comprises between 4 to up to 40 different modified RNA oligonucleotides. In some embodiments, the magnetic nanoparticle comprises between 5 to up to 40 different modified RNA oligonucleotides. In some embodiments, the magnetic nanoparticle comprises between 6 to up to 40 different modified RNA oligonucleotides. In some embodiments, the magnetic nanoparticle comprises between 7 to up to 40 different modified RNA oligonucleotides. In some embodiments, the magnetic nanoparticle comprises between 8 to up to 40 different modified RNA oligonucleotides. In some embodiments, the magnetic nanoparticle comprises between 9 to up to 40 different modified RNA oligonucleotides. In some embodiments, the magnetic nanoparticle comprises between 10 to up to 40 different modified RNA oligonucleotides. In some embodiments, the magnetic nanoparticle comprises between 11 to up to 40 different modified RNA oligonucleotides. In some embodiments, the magnetic nanoparticle comprises between 12 to up to 40 different modified RNA oligonucleotides. In some embodiments, the magnetic nanoparticle comprises between 13 to up to 40 different modified RNA oligonucleotides. In some embodiments, the magnetic nanoparticle comprises between 14 to up to 40 different modified RNA oligonucleotides. In some embodiments, the magnetic nanoparticle comprises between 15 to up to 40 different modified RNA oligonucleotides. In some embodiments, the magnetic nanoparticle comprises between 16 to up to 40 different modified RNA oligonucleotides. In some embodiments, the magnetic nanoparticle comprises between 17 to up to 40 different modified RNA oligonucleotides. In some embodiments, the magnetic nanoparticle comprises between 18 to up to 40 different modified RNA oligonucleotides. In some embodiments, the magnetic nanoparticle comprises between 19 to up to 40 different modified RNA oligonucleotides. In some embodiments, the magnetic nanoparticle comprises between 20 to up to 40 different modified RNA oligonucleotides. In some embodiments, the magnetic nanoparticle comprises between 21 to up to 40 different modified RNA oligonucleotides. In some embodiments, the magnetic nanoparticle comprises between 22 to up to 40 different modified RNA oligonucleotides. In some embodiments, the magnetic nanoparticle comprises between 23 to up to 40 different modified RNA oligonucleotides. In some embodiments, the magnetic nanoparticle comprises between 24 to up to 40 different modified RNA oligonucleotides. In some embodiments, the magnetic nanoparticle comprises between 25 to up to 40 different modified RNA oligonucleotides. In some embodiments, the magnetic nanoparticle comprises between 26 to up to 40 different modified RNA oligonucleotides. In some embodiments, the magnetic nanoparticle comprises between 27 to up to 40 different modified RNA oligonucleotides. In some embodiments, the magnetic nanoparticle comprises between 28 to up to 40 different modified RNA oligonucleotides. In some embodiments, the magnetic nanoparticle comprises between 29 to up to 40 different modified RNA oligonucleotides. In some embodiments, the magnetic nanoparticle comprises between 30 to up to 40 different modified RNA oligonucleotides. In some embodiments, the magnetic nanoparticle comprises between 31 to up to 40 different modified RNA oligonucleotides. In some embodiments, the magnetic nanoparticle comprises between 32 to up to 40 different modified RNA oligonucleotides. In some embodiments, the magnetic nanoparticle comprises between 33 to up to 40 different modified RNA oligonucleotides. In some embodiments, the magnetic nanoparticle comprises between 34 to up to 40 different modified RNA oligonucleotides. In some embodiments, the magnetic nanoparticle comprises between 35 to up to 40 different modified RNA oligonucleotides. In some embodiments, the magnetic nanoparticle comprises between 36 to up to 40 different modified RNA oligonucleotides. In some embodiments, the magnetic nanoparticle comprises between 37 to up to 40 different modified RNA oligonucleotides. In some embodiments, the magnetic nanoparticle comprises between 38 to up to 40 different modified RNA oligonucleotides. In some embodiments, the magnetic nanoparticle comprises between 39 to up to 40 different modified RNA oligonucleotides.

In some embodiments, the magnetic nanoparticle comprises at least 40 different modified RNA oligonucleotides. In some embodiments, the magnetic nanoparticle comprises at least 39 different modified RNA oligonucleotides. In some embodiments, the magnetic nanoparticle comprises at least 38 different modified RNA oligonucleotides. In some embodiments, the magnetic nanoparticle comprises at least 37 different modified RNA oligonucleotides. In some embodiments, the magnetic nanoparticle comprises at least 36 different modified RNA oligonucleotides. In some embodiments, the magnetic nanoparticle comprises at least 35 different modified RNA oligonucleotides. In some embodiments, the magnetic nanoparticle comprises at least 34 different modified RNA oligonucleotides. In some embodiments, the magnetic nanoparticle comprises at least 33 different modified RNA oligonucleotides. In some embodiments, the magnetic nanoparticle comprises at least 32 different modified RNA oligonucleotides. In some embodiments, the magnetic nanoparticle comprises at least 31 different modified RNA oligonucleotides. In some embodiments, the magnetic nanoparticle comprises at least 30 different modified RNA oligonucleotides. In some embodiments, the magnetic nanoparticle comprises at least 29 different modified RNA oligonucleotides. In some embodiments, the magnetic nanoparticle comprises at least 28 different modified RNA oligonucleotides. In some embodiments, the magnetic nanoparticle comprises at least 27 different modified RNA oligonucleotides. In some embodiments, the magnetic nanoparticle comprises at least 26 different modified RNA oligonucleotides. In some embodiments, the magnetic nanoparticle comprises at least 25 different modified RNA oligonucleotides. In some embodiments, the magnetic nanoparticle comprises at least 24 different modified RNA oligonucleotides. In some embodiments, the magnetic nanoparticle comprises at least 23 different modified RNA oligonucleotides. In some embodiments, the magnetic nanoparticle comprises at least 22 different modified RNA oligonucleotides. In some embodiments, the magnetic nanoparticle comprises at least 21 different modified RNA oligonucleotides. In some embodiments, the magnetic nanoparticle comprises at least 20 different modified RNA oligonucleotides. In some embodiments, the magnetic nanoparticle comprises at least 19 different modified RNA oligonucleotides. In some embodiments, the magnetic nanoparticle comprises at least 18 different modified RNA oligonucleotides. In some embodiments, the magnetic nanoparticle comprises at least 17 different modified RNA oligonucleotides. In some embodiments, the magnetic nanoparticle comprises at least 16 different modified RNA oligonucleotides. In some embodiments, the magnetic nanoparticle comprises at least 15 different modified RNA oligonucleotides. In some embodiments, the magnetic nanoparticle comprises at least 14 different modified RNA oligonucleotides. In some embodiments, the magnetic nanoparticle comprises at least 13 different modified RNA oligonucleotides. In some embodiments, the magnetic nanoparticle comprises at least 12 different modified RNA oligonucleotides. In some embodiments, the magnetic nanoparticle comprises at least 11 different modified RNA oligonucleotides. In some embodiments, the magnetic nanoparticle comprises at least 10 different modified RNA oligonucleotides. In some embodiments, the magnetic nanoparticle comprises at least 9 different modified RNA oligonucleotides. In some embodiments, the magnetic nanoparticle comprises at least 8 different modified RNA oligonucleotides. In some embodiments, the magnetic nanoparticle comprises at least 7 different modified RNA oligonucleotides. In some embodiments, the magnetic nanoparticle comprises at least 6 different modified RNA oligonucleotides. In some embodiments, the magnetic nanoparticle comprises at least 5 different modified RNA oligonucleotides. In some embodiments, the magnetic nanoparticle comprises at least 4 different modified RNA oligonucleotides. In some embodiments, the magnetic nanoparticle comprises at least 4 different modified RNA oligonucleotides. In some embodiments, the magnetic nanoparticle comprises at least 3 different modified RNA oligonucleotides. In some embodiments, the magnetic nanoparticle comprises at least 2 different modified RNA oligonucleotides.

In some embodiments, the pharmaceutical composition comprising at least one of the 5′pp or 5′ppp ss RNA oligonucleotide as described above and a pharmaceutically acceptable carrier. In some embodiments, the pharmaceutical composition further comprises an agent which facilitates the delivery of the oligonucleotide into a cell, in particular, into the cytosol of the cell. In some embodiments, the delivery agent is an agent described in herein (e.g., micelle, lipid nanoparticle (LNP), spherical nucleic acid (SNA), extracellular vesicle, synthetic vesicle, exosome, lipidoid, liposome, and lipoplex).

The pharmaceutical composition may further comprise another agent such as an agent that stabilizes the oligonucleotide. Examples of a stabilizing agent include a protein that complexes with the oligonucleotide to form an iRNP, chelators such as EDTA, salts, and RNase inhibitors.

In certain embodiments, the pharmaceutical composition, in particular, the pharmaceutical composition comprising a 5′pp or 5′ppp ss RNA oligonucleotide as described herein, further comprises one or more pharmaceutically active therapeutic agent(s). Examples of a pharmaceutically active agent include immunostimulatory agents, anti-viral agents, antibiotics, anti-fungal agents, anti-parasitic agents, anti-tumor agents, cytokines, chemokines, growth factors, anti-angiogenic factors, chemotherapeutic agents, antibodies and gene silencing agents. Preferably, the pharmaceutically active agent is selected from the group consisting of an immunostimulatory agent, an anti-viral agent and an anti-tumor agent. The more than one pharmaceutically active agents may be of the same or different category.

In certain embodiments, the pharmaceutical composition, in particular, the pharmaceutical composition comprising a 5′pp or 5′ppp ss RNA oligonucleotide as described herein, further comprises an antigen, an anti-viral vaccine, an anti-bacterial vaccine, and/or an anti-tumor vaccine, wherein the vaccine can be prophylactic and/or therapeutic.

In certain embodiments, the pharmaceutical composition, in particular, the pharmaceutical composition comprising a 5′pp or 5′ppp ss RNA oligonucleotide as described herein, further comprise retinoid acid, IFN-α and/or IFN-β. Without being bound by any theory, retinoid acid, IFN-α and/or IFN-β are capable of sensitizing cells for IFN-α production, possibly through the upregulation of RIG-I expression.

The pharmaceutical composition may be formulated in any way that is compatible with its therapeutic application, including intended route of administration, delivery format and desired dosage. Optimal pharmaceutical compositions may be formulated by a skilled person according to common general knowledge in the art.

The pharmaceutical composition may be formulated for instant release, controlled release, timed-release, sustained release, extended release, or continuous release.

The pharmaceutical composition may be administered by any route known in the art, including, but not limited to, topical, enteral and parenteral routes, provided that it is compatible with the intended application. Topical administration includes, but is not limited to, epicutaneous, inhalational, intranasal, vaginal administration, enema, eye drops, and ear drops. Enteral administration includes, but is not limited to, oral, rectal administration and administration through feeding tubes. Parenteral administration includes, but is not limited to, intravenous, intraarterial, intramuscular, intracardiac, subcutaneous, intraosseous, intradermal, intrathecal, intraperitoneal, transdermal, transmucosal, and inhalational administration. The pharmaceutical composition may be use for prophylactic and/or therapeutic purposes.

The optimal dosage, frequency, timing and route of administration can be readily determined by a person skilled in the art on the basis of factors such as the disease or condition to be treated, the severity of the disease or condition, the age, gender and physical status of the patient, and the presence or absence of previous treatment.

In some embodiments, the subject is administered at least one 5′pp or 5′ppp ss RNA oligonucleotide (e.g., RNA oligonucleotide as used herein) or pharmaceutical composition (e.g., any of the 5′pp or 5′ppp ss RNA oligonucleotides or pharmaceutical compositions described herein) and at least one additional therapeutic agent. The at least one additional therapeutic agent can be a chemotherapeutic agent (e.g., cyclophosphamide, mechlorethamine, chlorambucil, melphalan, daunorubicin, doxorubicin, epirubicin, idarubicin, mitoxantrone, valrubicin, paclitaxel, docetaxel, etoposide, teniposide, tafluposide, azacitidine, azathioprine, capecitabine, cytarabine, doxifluridine, fluorouracil, gemcitabine, mercaptopurine, methotrexate, tioguanine, bleomycin, carboplatin, cisplatin, oxaliplatin, bortezomib, carfilzomib, salinosporamide A, all-trans retinoic acid, vinblastine, vincristine, vindesine, and vinorelbine) and/or an analgesic (e.g., acetaminophen, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, phenylbutazone, piroxicam, sulindac, tolmetin, celecoxib, buprenorphine, butorphanol, codeine, hydrocodone, hydromorphone, levorphanol, meperidine, methadone, morphine, nalbuphine, oxycodone, oxymorphone, pentazocine, propoxyphene, and tramadol).

In some embodiments, the at least one additional therapeutic agent is an immunogenic cell death inducer (ICDi) (e.g., Daunorubicin, Docetaxel, Doxorubicin, Mitoxanthrone, Oxaliplatin, and Paclitaxel). In some embodiments, the at least one additional therapeutic agent is a siRNA therapy. In some embodiments, the siRNA therapy targets a gene associated with cancer (e.g., PD-L1, CTLA-4, TGF-β, and/or VEGF).

In some embodiments, the at least one additional therapeutic agent is a targeted therapy. Targeted therapies are a cornerstone of what is also referred to as precision medicine, a form of medicine that uses information about a person's genes and proteins to prevent, diagnose, and treat disease. Such therapeutics are sometimes called “molecularly targeted drugs,” or similar names. The process of discovering them is often referred to as “rational drug design.” This concept can also be referred to as “personalized medicine.”

Molecularly targeted drugs interact with a particular target molecule, or structurally related set of target molecules, in a pathway; thus modulating the endpoint effect of that pathway, such as a disease-related process; and, thus, yielding a therapeutic benefit.

Molecularly targeted drugs may be small molecules or biologics, usually antibodies. They may be useful alone or in combinations with other therapeutic agents and methods.

Because they target a particular molecule, or related set of molecules, and are usually designed to minimize their interactions with other molecules, targeted therapeutics may have fewer adverse side effects. Targeted cancer drugs block the growth and spread of cancer by interacting with specific molecules or sets of structurally related molecules (altogether, “molecular targets”) that are involved, broadly speaking, in the growth, progression, lack of suppression or elimination, or spread of cancer. Such molecular targets may include proteins or genes involved in one or more cellular functions including, for example and without limitation, signal transduction, gene expression modulation, apoptosis induction or suppression, angiogenesis inhibition, or immune system modulation.

Targeted therapy monoclonal antibodies (mAbs) and targeted small molecules are being used as treatments for cancer. They are used either as a monotherapy or in combination with other conventional therapeutic modalities, particularly if the disease under treatment is refractory to therapy using solely conventional techniques. In some embodiments, the at least one additional therapeutic agent is a molecularly targeted therapy. In some embodiments, the molecularly targeted therapy is selected from the group consisting of trastuzumab, gilotrif, proleukin, alectinib, campath, atezolizumab, avelumab, axitinib, belimumab, belinostat, bevacizumab, velcade, canakinumab, ceritinib, cetuximab, crizotinib, dabrafenib, daratumumab, dasatinib, denosumab, clotuzumab, enasidenib, erlotinib, gefitinib, ibrutinib, zydelig, imatinib, lenvatinib, midostaurin, necitumumab, niraparib, obinutuzumab, osimertinib, panitumumab, regorafenib, rituximab, ruxolitinib, sorafenib, tocilizumab, and trastuzumab.

In some embodiments, the at least one additional therapeutic agent is an immunotherapy. The term “immunotherapy,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non cytokine adjuvants. Alternatively the immunotherapeutic treatment may consist of administering the subject with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

In some embodiments, the immunotherapy is selected from the group consisting of pembrolizumab (Keytruda®), nivolumab (Opdivo®), atezolizumab (Tecentriq®), ipilimumab (Yervoy®), avelumab (Bavencio®) and durvalumab (Imfinzi®). In some embodiments, the subject has undergone or is undergoing an anti-PD-1, anti-PD-L1, or anti-CTLA4 therapy. Alternatively, any of the method may further comprise administering to the subject an effective amount of an anti-PD-1, anti-PD-L1, or anti-CTLA4 therapy. In some examples, the anti-PD-1, anti-PD-L1, or anti-CTLA4 therapy may comprise an anti-PD-1, anti-PD-L1, or anti-CTLA4 antibody, respectively. Exemplary anti-PD-1 antibodies include pembrolizumab, nivolumab, and AMP-224, or an antigen-binding fragment thereof. Exemplary anti-CTLA-4 antibodies include ipilimumab, and tremelimumab, or an antigen-binding fragment thereof. Exemplary anti-PD-L1 antibodies include durvalumab, atezolizumab, and avelumab, or an antigen-binding fragment thereof.

In some embodiments, at least one additional therapeutic agent and at least one 5′pp or 5′ppp ss RNA oligonucleotide (e.g., RNA oligonucleotide as used herein) are administered in the same composition (e.g., the same pharmaceutical composition). By “at least one”, it is meant that one or more 5′pp or 5′ppp ss RNA oligonucleotide(s) of the same or different oligonucleotide(s) can be used together.

In some embodiments, the at least one additional therapeutic agent and the at least one 5′pp or 5′ppp ss RNA oligonucleotide are administered to the subject using different routes of administration (e.g., at least one additional therapeutic agent delivered by oral administration and at least one 5′pp or 5′ppp ss RNA oligonucleotide delivered by intravenous administration).

In any of the methods described herein, the at least one 5′pp or 5′ppp ss RNA oligonucleotide or pharmaceutical composition (e.g., any of the 5′pp or 5′ppp ss RNA oligonucleotides or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different 5′pp or 5′ppp ss RNA oligonucleotides are administered in the same composition (e.g., a liquid composition). In some embodiments, the at least one 5′pp or 5′ppp ss RNA oligonucleotide and the at least one additional therapeutic agent are administered in the same composition (e.g., a liquid composition). In some embodiments, the at least one 5′pp or 5′ppp ss RNA oligonucleotide and the at least one additional therapeutic agent are administered in two different compositions (e.g., a liquid composition containing at least one 5′pp or 5′ppp ss RNA oligonucleotide and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to administering the at least one 5′pp or 5′ppp ss RNA oligonucleotide or pharmaceutical composition (e.g., any of the 5′pp or 5′ppp ss RNA oligonucleotide or pharmaceutical compositions described herein). In some embodiments, the one or more additional therapeutic agents can be administered to the subject after administering the at least one 5′pp or 5′ppp ss RNA oligonucleotide or pharmaceutical composition (e.g., any of the magnetic particles or pharmaceutical compositions described herein). In some embodiments, the one or more additional therapeutic agents and the at least one 5′pp or 5′ppp ss RNA oligonucleotide or pharmaceutical composition (e.g., any of the 5′pp or 5′ppp ss RNA oligonucleotides or pharmaceutical compositions described herein) are administered to the subject such that there is an overlap in the bioactive period of the one or more additional therapeutic agents and the at least one 5′pp or 5′ppp ss RNA oligonucleotide (e.g., any of the 5′pp or 5′ppp ss RNA oligonucleotides described herein) in the subject.

In some embodiments, the subject can be administered the at least one 5′pp or 5′ppp ss RNA oligonucleotide or pharmaceutical composition (e.g., any of the 5′pp or 5′ppp ss RNA oligonucleotides or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, or 10 years). A skilled medical professional may determine the length of the treatment period using any of the methods described herein for diagnosing or following the effectiveness of treatment (e.g., using the methods above and those known in the art). As described herein, a skilled medical professional can also change the identity and number (e.g., increase or decrease) of 5′pp or 5′ppp ss RNA oligonucleotides (and/or one or more additional therapeutic agents) administered to the subject and can also adjust (e.g., increase or decrease) the dosage or frequency of administration of at least one 5′pp or 5′ppp ss RNA oligonucleotide (and/or one or more additional therapeutic agents) to the subject based on an assessment of the effectiveness of the treatment (e.g., using any of the methods described herein and known in the art). A skilled medical professional can further determine when to discontinue treatment (e.g., for example, when the subject's symptoms are significantly decreased).

### 7. Delivery

The 5′pp or 5′ppp ss RNA oligonucleotide (e.g., RNA oligonucleotide as used herein) can be delivered to a host cell or subject, in vivo or ex vivo, using various known and suitable methods available in the art. As provided herein, delivery systems including lipoplexes, liposomes, lipid nanoparticles (LNPs), spherical nucleic acids (SNAs), nanoparticles, and other methods known in the art may be used for delivery of the 5′pp or 5′ppp ss RNA oligonucleotide.

Various delivery systems (e.g., liposomes, nanoparticles) containing the 5′pp or 5′ppp ss RNA oligonucleotide can also be administered to an organism for delivery to cells in vivo or administered to a cell or cell culture ex vivo. Administration is by any of the routes normally used for introducing a molecule into ultimate contact with blood, fluid, or cells including, but not limited to, injection, infusion, topical application and electroporation. Suitable methods of administering such oligonucleotides are available and well known to those of skill in the art.

Oligonucleotide Delivery Strategies

The oligonucleotide therapeutics field has seen remarkable progress over the last few years. However, effective delivery of oligonucleotides to their intracellular sites of action remains a major issue. The biological basis of oligonucleotide delivery includes the nature of various tissue barriers and the mechanisms of cellular uptake and intracellular trafficking of oligonucleotides. The current approaches for enhancing the delivery of oligonucleotides include molecular scale targeted ligand-oligonucleotide conjugates, lipid- and polymer-based nanoparticles, spherical nucleic acids (inorganic nanoparticles coated with nucleic acids), micelles, extracellular vesicles, synthetic vesicles, exosome, lipidoid, antibody conjugates and small molecules that improve oligonucleotide delivery. The merits and liabilities of these approaches are placed in the context of the underlying basic biology. Some of these methods of delivery are described in more detail below.

Lipoplexes, Liposomes, and Lipid Nanoparticles

Formulation with lipids is one of the most common approaches to enhancing nucleic acid delivery. Mixing polyanionic nucleic acid drugs with lipids leads to the condensing of nucleic acids into nanoparticles that have a more favorable surface charge, and are sufficiently large (˜100 nm in diameter) to trigger uptake by endocytosis. Lipoplexes are the result of direct electrostatic interaction between polyanionic nucleic acid and the cationic lipid, and are typically a heterogeneous population of relatively unstable complexes. Lipoplex formulations need to be prepared shortly before use, and have been successfully used for local delivery applications. By contrast, liposomes comprise a lipid bilayer, with the nucleic acid drug residing in the encapsulated aqueous space. Liposomes are more complex (typically consisting of cationic or fusogenic lipids [to promote endosomal escape] and cholesterol PEGylated lipid) and exhibit more consistent physical properties with greater stability than lipoplexes. For example, some lipid nanoparticles (LNPs), also known as stable nucleic acid lipid particles, are liposomes that contain ionizable lipid, phosphatidylcholine, cholesterol and PEG-lipid conjugates in defined ratios and have been successfully utilized in multiple instances. Landmark examples are the silencing of hepatitis B virus and APOB by siRNAs in preclinical animal studies and, more recently, the approval of patisiran, an siRNA that is delivered as an LNP formulation. Encapsulation of nucleic acid cargos provides a means of protection from nuclease digestion in the circulation and in the endosome. Additionally, ionizable LNPs also associate with APOE, which further facilitates liver uptake via LDLR-mediated endocytosis. Similarly, LNPs containing lipidoid or lipid-like materials have demonstrated robust siRNA-mediated silencing in rodents and non-human primates.

Lipid nanoparticles (LNPs) are a well-known means for delivery of nucleotide cargo, and may be used for delivery of the 5′pp or 5′ppp ss RNA oligonucleotides disclosed herein. A disadvantage of LNPs is that their delivery is primarily limited to the liver and reticuloendothelial system as the sinusoidal capillary epithelium in this tissue provides spaces large enough to allow the entry of these relatively large nanoparticles. However, local delivery of LNPs has been used to successfully deliver siRNAs to the CNS after intracerebroventricular injection. Conversely, the large size of nanoparticles is advantageous as it essentially precludes renal filtration and permits delivery of a higher payload.

In some embodiments, provided herein is a method for delivering the 5′pp or 5′ppp ss RNA oligonucleotides disclosed herein to a host cell or subject, wherein the 5′pp or 5′ppp ss RNA oligonucleotides are delivered via an LNP. In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of PCT/US2018/053559, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the 5′pp or 5′ppp ss RNA oligonucleotides disclosed herein is formulated in or administered via a lipid nanoparticle; see e.g., WO/2017/173054, the contents of which are hereby incorporated by reference in their entirety. Any of the 5′pp or 5′ppp ss RNA oligonucleotides described herein may be delivered by LNP. In some instances, the lipid component comprises a biodegradable, ionizable lipid, cholesterol, DSPC, and PEG-DMG.

Spherical Nucleic Acids (SNA)

An alternative nanoparticle-based delivery strategy is the SNA approach. SNA particles consist of a hydrophobic core nanoparticle (comprising gold, silica or various other materials) that is decorated with hydrophilic oligonucleotides (for example, ASOs, siRNAs and immunostimulatory oligonucleotides) that are densely packed onto the surface via thiol linkages. In contrast to other nanoparticle designs, SNA-attached oligonucleotides radiate outwards from the core structure. While exposed, the oligonucleotides are protected from nucleolytic degradation to some extent as a consequence of steric hindrance, high local salt concentration, and through interactions with corona proteins.

Nanoparticles

In some embodiments, the 5′pp or 5′ppp ss RNA oligonucleotides are linked or conjugated to nanoparticles, e.g., as described in WO2013/016126. In some embodiments, the nanoparticles have a diameter of between about 2 nm to about 200 nm (e.g., between about 10 nm to about 30 nm, between about 5 nm to about 25 nm, between about 10 nm to about 25 nm, between about 15 nm to about 25 nm, between about 20 nm and about 25 nm, between about 25 nm to about 50 nm, between about 50 nm and about 200 nm, between about 70 nm and about 200 nm, between about 80 nm and about 200 nm, between about 100 nm and about 200 nm, between about 140 nm to about 200 nm, and between about 150 nm to about 200 nm), and contain a polymer coating.

In some embodiments, the nanoparticles provided herein can be spherical or ellipsoidal, or can have an amorphous shape. In some embodiments, the nanoparticles provided herein can have a diameter (between any two points on the exterior surface of the nanoparticle) of between about 2 nm to about 200 nm (e.g., between about 10 nm to about 200 nm, between about 2 nm to about 30 nm, between about 5 nm to about 25 nm, between about 10 nm to about 25 nm, between about 15 nm to about 25 nm, between about 20 nm to about 25 nm, between about 50 nm to about 200 nm, between about 70 nm to about 200 nm, between about 80 nm to about 200 nm, between about 100 nm to about 200 nm, between about 140 nm to about 200 nm, and between about 150 nm to about 200 nm). In some embodiments, nanoparticles having a diameter of between about 2 nm to about 30 nm localize to the lymph nodes in a subject. In some embodiments, nanoparticles having a diameter of between about 40 nm to about 200 nm localize to the liver.

In some embodiments, the nanoparticles described herein do not contain a magnetic material. In some embodiments, a nanoparticle can contain, in part, a core of containing a polymer (e.g., poly(lactic-co-glycolic acid)). Skilled practitioners will appreciate that any number of art known materials can be used to prepare nanoparticles, including, but are not limited to, gums (e.g., Acacia, Guar), chitosan, gelatin, sodium alginate, and albumin. Additional polymers that can be used to generate the nanoparticles described herein are known in the art. For example, polymers that can be used to generate the nanoparticles include, but are not limited to, cellulosics, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polycyanoacrylate and polycaprolactone.

Skilled practitioners will appreciate that the material used in the composition of the nanoparticles, the methods for preparing, coating, and methods for controlling the size of the nanoparticles can vary substantially. However, these methods are well known to those in the art. Key issues include the biodegradability, toxicity profile, and pharmacokinetics/pharmacodynamics of the nanoparticles. The composition and/or size of the nanoparticles are key determinants of their biological fate. For example, larger nanoparticles are typically taken up and degraded by the liver, whereas smaller nanoparticles (<30 nm in diameter) typically circulate for a long time (sometimes over 24-hr blood half-life in humans) and accumulate in lymph nodes and the interstitium of organs with hyperpermeable vasculature, such as tumors.

Magnetic Nanoparticles

In some embodiments, the nanoparticles can be magnetic (e.g., contain a core of a magnetic material). In some embodiments, the magnetic nanoparticles include ferric chloride, ferrous chloride, or a combination thereof, and a dextran coating. In some embodiments, the magnetic nanoparticles contain a mixture of two or more of the different nanoparticle compositions described herein. In some embodiments, the compositions contain at least one magnetic nanoparticle having a tunable surface functionalization, and at least one magnetic nanoparticle having tunable magnetic properties.

In some embodiments, any of the nanoparticles described herein can contain a core of a magnetic material (e.g., a therapeutic magnetic nanoparticle). In some embodiments, the magnetic material or particle can contain a diamagnetic, paramagnetic, superparamagnetic, or ferromagnetic material that is responsive to a magnetic field. Non-limiting examples of therapeutic magnetic nanoparticles contain a core of a magnetic material containing a metal oxide selected from the group of: magnetite; ferrites (e.g., ferrites of manganese, cobalt, and nickel); Fe(II) oxides, and hematite, and metal alloys thereof. The core of magnetic material can be formed by converting metal salts to metal oxides using methods known in the art (e.g., Kieslich et al., Inorg. Chem. 2011). In some embodiments, the nanoparticles contain cyclodextrin gold or quantum dots. Non-limiting examples of methods that can be used to generate therapeutic magnetic nanoparticles are described in Medarova et al., Methods Mol. Biol. 555:1-13, 2009; and Medarova et al., Nature Protocols 1:429-431, 2006. Additional magnetic materials and methods of making magnetic materials are known in the art. In some embodiments of the methods described herein, the position or localization of therapeutic magnetic nanoparticles can be imaged in a subject (e.g., imaged in a subject following the administration of one or more doses of a therapeutic magnetic nanoparticle).

In some embodiments, the magnetic nanoparticles can be functionalized with one or more amine groups. In some embodiments, the functionalization occurs at the surface of the magnetic nanoparticles. In some embodiments, the one or more amine groups are covalently linked to the dextran coating. In some embodiments, the one or more amine groups substitute one or more hydroxyl groups of the dextran coating. In some embodiments, the number of the one or more amine groups is tunable based on a concentration of ferric chloride, ferrous chloride, or a combination thereof. In some embodiments, the nanoparticle composition includes about 5 to about 1000 amine groups. In some embodiments, the nanoparticle composition includes about 5 to 25, 25 to 100, 100 to 150, 150 to 200, 200 to 250, 250 to 300, 300 to 350, 350 to 400, 450 to 500, 500 to 550, 550 to 600, 600 to 650, 650 to 700, 700 to 750, 750 to 800, 800 to 850, 850 to 900, 900 to 950, or 950 to 1000 amine groups.

In some embodiments, the magnetic nanoparticles can contain a core of a magnetic material (e.g., ferric chloride and/or ferrous chloride). In some embodiments, the magnetic nanoparticles include about 0.60 g to about 0.70 g of ferric chloride and about 0.3 g to about 0.5 g of ferrous chloride. In some embodiments, the magnetic nanoparticles including about 0.60 g to about 0.70 g of ferric chloride and about 0.3 g to about 0.5 g of ferrous chloride are functionalized with about 5 to 150 amine groups. In some embodiments, the magnetic nanoparticles including about 0.65 g of ferric chloride and about 0.4 g of ferrous chloride are functionalized with about 60 to 90 amine groups. In some embodiments, the magnetic nanoparticles including about 0.65 g of ferric chloride and about 0.4 g of ferrous chloride are functionalized with about 5 to 150 amine groups. In some embodiments, the magnetic nanoparticles including about 0.65 g of ferric chloride and about 0.4 g of ferrous chloride are functionalized with about 1 to 150 amine groups. In some embodiments, the magnetic nanoparticles including about 0.65 g of ferric chloride and about 0.4 g of ferrous chloride are functionalized with about at least 1 to 10 amine groups, 10 to 20 amine groups, about 20 to 30 amine groups, about 30 to 40 amine groups, about 40 to 50 amine groups, about 50 to 60 amine groups, about 60 to 70 amine groups, about 70 to 80 amine groups, about 80 to 90 amine groups, about 90 to 100 amine groups, about 100 to 110 amine groups, about 110 to 120 amine groups, about 120 to 130 amine groups, about 130 to 140 amine groups, or about 140 to 150 amine groups.

In some embodiments, the magnetic nanoparticles include about 1 g to about 1.4 g of ferric chloride. In some embodiments, the magnetic nanoparticles including about 1 g to about 1.4 g of ferric chloride are functionalized with about 246 to 500 amine groups. In some embodiments, the magnetic nanoparticles including about 1.2 g of ferric chloride are functionalized with about 246 to 500 amine groups. In some embodiments, the magnetic nanoparticles functionalized with about 246 to 500 amine groups do not include ferric chloride. In some embodiments, the magnetic nanoparticles including about 1.2 g of ferric chloride are functionalized with about 200 to 600 amine groups. In some embodiments, the magnetic nanoparticles including about 1.2 g of ferric chloride are functionalized with about at least 200 to 250 amine groups, 250 to 300 amine groups, about 300 to 350 amine groups, about 350 to 400 amine groups, about 400 to 450 amine groups, about 450 to 500 amine groups, about 500 to 550 amine groups, about 550 to 600 amine groups, or more.

Thus, in some embodiments, the number of amine groups conjugated to the dextran coating can be fine-tuned by controlling the concentrations of ferric chloride and ferrous chloride, which are used to prepare the magnetic nanoparticles.

In some embodiments, the magnetic nanoparticles include magnetic nanoparticles having a magnetic strength that is tunable based on a concentration of ferric chloride, ferrous chloride, or a combination thereof.

In some embodiments, the magnetic nanoparticles include about 0.1% to about 99.9% of ferric ion and about 99.9% to about 0.1% of ferrous ion in total iron per MNP. In some embodiments, the magnetic nanoparticles including about 60% to about 80% of ferric chloride and about 20% to about 40% of ferrous chloride have stronger magnetic properties than nanoparticle compositions having a ferrous chloride amount higher than about 80%. In some embodiments, the magnetic nanoparticles including about 70% of ferric ion and about 30% g of ferrous ion have stronger magnetic properties than magnetic nanoparticles having a ferrous ion amount higher than about 30%.

In some embodiments, the magnetic nanoparticles have a non-linearity index (NLI) ranging from about 6 to about 40. In some embodiments, the magnetic nanoparticles have an NLI ranging from about 6 to about 70. In some embodiments, the magnetic nanoparticles have an NLI ranging from about 8.5 to about 14.8. In some embodiments, the magnetic nanoparticles have an NLI ranging from about 8 to about 14. In some embodiments, the magnetic nanoparticles have an NLI of about 6. In some embodiments, the magnetic nanoparticles have an NLI of about 8. In some embodiments, the magnetic nanoparticles have an NLI of about 14. In some embodiments, the magnetic nanoparticles have an NLI of about 67. In some embodiments, the magnetic nanoparticles have an NLI ranging from 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, 20 to 30, 30 to 40, 40 to 50, 50 to 60, or 60 to 70. In some embodiments, the magnetic nanoparticles including about 0.54 g of ferric chloride and about 0.2 g of ferrous chloride have a NLI ranging from about 8.5 to about 14.8. In some embodiments, the magnetic nanoparticles including about 0.54 g of ferric chloride and about 0.2 g of ferrous chloride have a NLI of about 12. In some embodiments, the magnetic strength of the magnetic nanoparticles can be quantified by measuring a non-linearity index (NLI) by magnetic particle spectrometry as described in WO 2021/113829.

In some embodiments, the magnetic nanoparticles include about 80% to about 100% of ferric chloride and about 20% to about 0% of ferrous chloride. In some embodiments, the magnetic nanoparticles including about 0% to about 50% of ferric chloride and about 100% to about 50% of ferrous chloride have weaker magnetic properties than magnetic nanoparticles having a ferrous chloride amount lower than about 0.4 g. In some embodiments, the magnetic nanoparticles including about 0.54 g of ferric chloride and about 0.4 g of ferrous chloride have weaker magnetic properties than magnetic nanoparticles having a ferrous chloride amount lower than about 0.2 g. In some embodiments, the magnetic nanoparticles including about 0.54 g of ferric chloride and about 0.4 g of ferrous chloride have a NLI ranging from about 50 to about 120. In some embodiments, the magnetic nanoparticles including about 0.54 g of ferric chloride and about 0.4 g of ferrous chloride have a NLI of about 67.

Thus, in some embodiments, the magnetic properties (e.g., magnetic strength) of the magnetic nanoparticles can be fine-tuned by controlling the concentrations of ferric chloride and ferrous chloride, which are used to prepare the magnetic nanoparticles.

In some embodiments, the magnetic nanoparticles has an iron concentration ranging from about 8 mM to about 217 mM. In some embodiments, the magnetic nanoparticles has an iron concentration ranging from about 8 mM to about 15 mM, about 15 mM to about 25 mM, about 25 mM to about 50 mM, 50 mM to about 60 mM, about 60 mM to about 70 mM, about 70 mM to about 80 mM, about 80 mM to about 90 mM, about 90 mM to about 100 mM, about 100 mM to about 110 mM, about 110 mM to about 120 mM, about 120 mM to about 130 mM, about 130 mM to about 140 mM, about 140 mM to about 150 mM, about 150 mM to about 160 mM, about 160 mM to about 170 mM, about 170 mM to about 180 mM, about 180 mM to about 190 mM, about 190 mM to about 200 mM, about 200 mM to about 210 mM, and about 210 mM to about 220 mM.

In some embodiments, the magnetic nanoparticles have an iron concentration ranging from about 1 mg/mL to about 25 mg/mL. In some embodiments, the magnetic nanoparticles has an iron concentration ranging from about 1 mg/mL to about 5 mg/mL, about 5 mg/mL to about 10 mg/mL, about 10 mg/mL to about 15 mg/mL, about 15 mg/mL to about 20 mg/mL, or about 20 mg/mL to about 25 mg/mL.

In some embodiments, the magnetic nanoparticles are used to deliver a composition containing at least one (e.g., one, two, three, or four) of any of the 5′pp or 5′ppp ss RNA oligonucleotides (e.g., RNA oligonucleotide as used herein) described herein. By “at least one”, it is meant that one or more 5′pp or 5′ppp ss RNA oligonucleotide(s) of the same or different oligonucleotide(s) can be used together. In some embodiments, the magnetic nanoparticle delivers one 5′pp or 5′ppp ss RNA oligonucleotide. In some embodiments, the magnetic nanoparticle delivers two 5′pp or 5′ppp ss RNA oligonucleotides. In some embodiments, the magnetic nanoparticle delivers three 5′pp or 5′ppp ss RNA oligonucleotides. In some embodiments, the magnetic nanoparticle delivers four 5′pp or 5′ppp ss RNA oligonucleotides. In some embodiments, the magnetic nanoparticle delivers five 5′pp or 5′ppp ss RNA oligonucleotides.

Polymer Coatings of Nanoparticles

In some embodiments, the nanoparticles described herein contain a polymer coating over the core magnetic material (e.g., over the surface of a magnetic material). The polymer material can be suitable for attaching or coupling one or more biological agents (e.g., such as any of the nucleic acids, fluorophores, or targeting peptides described herein). One of more biological agents (e.g., a nucleic acid, fluorophore, or targeting peptide) can be fixed to the polymer coating by chemical coupling (covalent bonds).

In some embodiments, the nanoparticles are formed by a method that includes coating the core of magnetic material with a polymer that is relatively stable in water. In some embodiments, the nanoparticles are formed by a method that includes coating a magnetic material with a polymer or absorbing the magnetic material into a thermoplastic polymer resin having reducing groups thereon. A coating can also be applied to a magnetic material using the methods described in U.S. Pat. Nos. 5,834,121, 5,395,688, 5,356,713, 5,318,797, 5,283,079, 5,232,789, 5,091,206, 4,965,007, 4,774,265, 4,770,183, 4,654,267, 4,554,088, 4,490,436, 4,336,173, and 4,421,660; and WO 10/111066 (each disclosure of which is incorporated herein by reference).

Methods for the synthesis of iron oxide nanoparticles include, for example, physical and chemical methods. For example, iron oxides can be prepared by co-precipitation of Fe2+ and Fe3+ salts in an aqueous solution. The resulting core consists of magnetite (Fe3O4), maghemite (γ-Fe2O3) or a mixture of the two. The anionic salt content (chlorides, nitrates, sulphates etc), the Fe2+ and Fe3+ ratio, pH and the ionic strength in the aqueous solution all play a role in controlling the size. It is important to prevent the oxidation of the synthesized nanoparticles and protect their magnetic properties by carrying out the reaction in an oxygen free environment under inert gas such as nitrogen or argon. The coating materials can be added during the co-precipitation process in order to prevent the agglomeration of the iron oxide nanoparticles into microparticles. Skilled practitioners will appreciated that any number of art known surface coating materials can be used for stabilizing iron oxide nanoparticles, among which are synthetic and natural polymers, such as, for example, polyethylene glycol (PEG), dextran, polyvinylpyrrolidone (PVP), fatty acids, polypeptides, chitosin, and/or gelatin.

For example, U.S. Pat. No. 4,421,660 note that polymer coated particles of an inorganic material are conventionally prepared by (1) treating the inorganic solid with acid, a combination of acid and base, alcohol or a polymer solution; (2) dispersing an addition polymerizable monomer in an aqueous dispersion of a treated inorganic solid and (3) subjecting the resulting dispersion to emulsion polymerization conditions. (col. 1, lines 21-27) U.S. Pat. No. 4,421,660 also discloses a method for coating an inorganic nanoparticles with a polymer, which comprises the steps of (1) emulsifying a hydrophobic, emulsion polymerizable monomer in an aqueous colloidal dispersion of discrete particles of an inorganic solid and (2) subjecting the resulting emulsion to emulsion polymerization conditions to form a stable, fluid aqueous colloidal dispersion of the inorganic solid particles dispersed in a matrix of a water-insoluble polymer of the hydrophobic monomer (col. 1, lines 42-50).

Alternatively, polymer-coated magnetic material can be obtained commercially that meets the starting requirements of size. For example, commercially available ultra-small superparamagnetic iron oxide nanoparticles include NC100150 Injection (Nycomed Amersham, Amersham Health) and Ferumoxytol (AMAG Pharmaceuticals, Inc.).

Suitable polymers that can be used to coat the core of magnetic material include without limitation: polystyrenes, polyacrylamides, polyetherurethanes, polysulfones, fluorinated or chlorinated polymers such as polyvinyl chloride, polyethylenes, and polypropylenes, polycarbonates, and polyesters. Additional examples of polymers that can be used to coat the core of magnetic material include polyolefins, such as polybutadiene, polydichlorobutadiene, polyisoprene, polychloroprene, polyvinylidene halides, polyvinylidene carbonate, and polyfluorinated ethylenes. A number of copolymers, including styrene/butadiene, alpha-methyl styrene/dimethyl siloxane, or other polysiloxanes can also be used to coat the core of magnetic material (e.g., polydimethyl siloxane, polyphenylmethyl siloxane, and polytrifluoropropylmethyl siloxane). Additional polymers that can be used to coat the core of magnetic material include polyacrylonitriles or acrylonitrile-containing polymers, such as poly alpha-acrylanitrile copolymers, alkyd or terpenoid resins, and polyalkylene polysulfonates. In some embodiments, the polymer coating is dextran.

### EXEMPLIFICATION

The invention now being generally described, it will be more readily understood by reference to the following examples, which are included merely for purposes of illustration of certain embodiments of the present invention, and are not intended to limit the invention.

### Example 1. Design, Synthesis, and Testing of RNA Oligonucleotides

In this example, miRNA inhibitors are designed to fully complementary to their target miRNAs with modifications at 5′ end, biphophate (pp) triphosphate (ppp) for potent agonist response, and 3′ end, thio-MC6-D for conjugation to magnetic nanoparticles (MN) for delivery. 5′pp or 5′ppp modification will be omitted for control oligo. Blunt-ended double-stranded structures are generated by annealing 5′pp or 5′ppp-anti-miRNA-3-thio-MC6-D with complementary miRNA, which can also be conjugated to MN. All custom RNA oligonucleotides are synthesized using known methods.

Synthesis and Characterization of Nano-Conjugates

The procedure is adapted from a publication (Medarova Z. et al., 2016. Controlling RNA Expression in Cancer Using Iron Oxide Nanoparticles Detectable by MRI and In Vivo Optical Imaging. Methods Mol Biol. 2016; 1372:163-179) and briefly summarized below. The disulfide on the oligonucleotide is activated by 3% Tris (2-carboxyethyl) phosphine hydrochloride (TCEP, Thermo Scientific Co., Rockford, IL), followed by purification with ammonium acetate/ethanol precipitation treatment prior to conjugation to the nanoparticles. Aminated magnetic nanoparticles are synthesized. Nanoparticles with a size of 20+nm are used for conjugation to the oligonucleotides. The magnetic nanoparticles are conjugated to the hetero-bifunctional linker N-succinimidyl 3-[2-pyridyldithio]-propionate (SPDP; Thermo Scientific Co., Rockford, IL) and activated oligonucleotides sequentially. Briefly, SPDP is dissolved in anhydrous DMSO and incubated with magnetic nanoparticles. The 3′-ThioMC6 of the oligo is activated to release the thiol via 3% TCEP treatment in nuclease-free PBS. The oligonucleotides are purified using an ammonium acetate/ethanol precipitation method. After TCEP activation and purification, the oligonucleotides are dissolved in water and incubated with the SPDP-modified magnetic nanoparticles overnight. The number of oligonucleotides per magnetic nanoparticle is determined using the electrophoresis analysis method.

### Example 2. Protein Expression and Purification

Full-length human RIG-I is cloned in Escherichia coli and expressed in a recombinant form with a His-SUMO tag as reported (Kwok J. et al. 2014. Expression, purification, crystallization and preliminary X-ray analysis of full-length human RIG-I. Acta Crystallogr F Struct Biol Commun. 70(Pt 2):248-251). The protein expression and purification is adapted and modified from a published procedure, as summarized below (Rawling D C. et al. 2020. Small-Molecule Antagonists of the RIG-I Innate Immune Receptor. ACS Chemical Biology. 15(2):311-317.). The RIG-I expression plasmid is transformed into Rosetta II(DE3) Escherichia coli cells (Novagen) using 150 ng/25 uL commercial cell stocks and grown in LB media supplemented with 50 mM Potassium Phosphate pH 7.4 and 1% glycerol. Expression is induced by the addition of isopropyl-β-D-thiogalactopyranoside (IPTG) to a final concentration of 0.5 mM. Cells are grown for 24 h at 16° C., then harvested by centrifugation, resuspended in lysis buffer (20 mM Phosphate pH 7.4, 500 mM NaCl, 10% glycerol, 5 mM β-mercaptoethanol (βME)) to a final volume of 50 ml and frozen at −80° C. For lysis, frozen pellets are thawed at room temperature, then resuspended in an additional 200 ml lysis buffer per 4L pellet. Cells are lysed by passage through a microfluidizer at 15,000 psi or method of choice, and the lysate is clarified by ultracentrifugation at 100,000×g for 30 min. Soluble lysate is incubated on 2.5 ml Ni-NTA beads (Qiagen), washed with lysis buffer containing an additional 40 mM imidazole, then eluted in Ni elution buffer (25 mM HEPES pH 8.0, 150 mM NaCl. 220 mM Imidazole, 10% glycerol, 5 mM PME). Eluted protein is bound to a HiTrap Heparin HP column (GE Biosciences), washed in buffer containing 150 mM NaCl and eluted stepwise at 0.65 M NaCl. The SUMO tag is then removed by incubation with SUMO protease for 2 h at 4° C. Finally, monomeric protein is collected by passage over a HiPrep 16/60 Superdex 200 column (GE Biosciences) in gel filtration buffer (25 mM MOPS pH 7.4, 300 mM NaCl, 5% glycerol, 5 mM PME). Peak fractions are concentrated to 10-20 μM using a centrifugal concentrator with a 50 kD molecular weight cutoff (Millipore).

### Example 3. In Vitro Study of RIG-I Activation by 5′Pp- or 5′Ppp-ds-miRNA Mimics

An ATP/NADH coupled assay for ATPase activity is based on a reaction in which the regeneration of hydrolyzed ATP is coupled to the oxidation of NADH. Following each cycle of ATP hydrolysis, the regeneration system consisting of phosphoenolpyruvate (PEP) and pyruvate kinase (PK) converts one molecule of PEP to pyruvate when the ADP is converted back to the ATP. The pyruvate is subsequently converted to lactate by lactate dehydrogenase (LDH) resulting in the oxidation of one NADH molecule. The assay measures the rate of NADH absorbance decrease at 340 nm, which is proportional to the rate of steady-state ATP hydrolysis. The constant regeneration of ATP allows monitoring the ATP hydrolysis rate over the entire course of the assay. A 96-well microplate format reader permits the simultaneous analysis of up to 96 samples. RIG-I is an ATP-dependent RNA helicase. Binding and activation by 5′pp or 5′ppp-ds-miRNA mimic confers ATPase activity on this protein. The enzyme assay can be used conveniently to test and/or screen agonists or antagonist for RIG-I receptor.

Example procedure for NADH-coupled ATPase assay is described below (Rawling D C. et al. 2020. Small-Molecule Antagonists of the RIG-I Innate Immune Receptor. ACS Chemical Biology. 15(2):311-317). For the NADH-coupled assay, RIG-I protein is diluted into ATPase assay buffer (25 mM MOPS pH 7.4, 150 mM KCl, 2 mM DTT) to a final concentration of 10 nM for early compounds and then 20 nM for visualizing more potent inhibitors. In this case, RIG-I wis activated by the desired RNA oligo or control which is added to a final concentration of 250 nM. A coupled assay mixture consisting of 1 mM NADH, 100 U/mi lactic dehydrogenase, 500 U/ml pyruvate kinase, 2.5 mM phospho enol pyruvic acid is added to the sample. Samples are incubated for at least 1 hour at RT. Reactions are initiated by the addition of a 1:1 ATP/MgCl2 mix to a final concentration of 5 mM.

RNA agonist-induced RIG-I activation is evaluated by measuring type I interferon using cell-based reporter gene assays based on readily available cell lines. Commercial cell lines developed for reporter gene assays that are responsive to IFN exposure are increasingly available. These cells produce a soluble gene product that can be readily quantified using multi-well plate spectrophotometers or luminometers.

The InvivoGen HEK-Lucia™ RIG-I cells were generated from HEK-Lucia™ Null cells, HEK293-derived cells that stably express the secreted Lucia luciferase reporter gene. This reporter gene is under the control of an IFN-inducible ISG54 promoter enhanced by a multimeric IFN-stimulated response elements (ISRE). HEK-Lucia™ RIG-I cells stably express high levels of human RIG-land respond strongly to cytosolic double-stranded RNAs with an uncapped 5′-triphosphate end such as 3p-hpRNA and 5′pp or 5′ppp-dsRNA. HEK-Lucia™ RIG-I and HEK-Lucia™ Null cells can be used to study the role of RIG-I by monitoring IRF-induced Lucia luciferase activity. The levels of IRF-induced Lucia in cell culture supernatants can be easily monitored using QUANTI-Luc™, a Lucia luciferase detection reagent (also from InvivoGen). To achieve stimulation of RIG-I using naked 5′pp or 5′ppp-dsRNA or controls that need to be delivered into the cytoplasm, a transfection agent, such as LyoVec™ (InvivoGen) can be used.

### Example 4. Animal Models

Featuring tissue-specific tumor implantation that is easily monitored with in-life imaging techniques, orthotopic models create a disease-relevant tumor microenvironment (TME) for better translation into the clinic. Orthotopic models involve the seeding of tumor cell lines into the corresponding tissue in animal models. This strategy allows us to assess tumor development in a relevant environment and evaluate efficacy in a preclinical tumor model that mimics the disease process in humans. With orthotopic models, disease progression is monitored through a variety of methods, including clinical signs, survival study design, and imaging platform that has both in vivo and ex vivo capabilities. Examples of the study designs are described below.

Exemplary metastatic breast cancer cell lines that can be used include MDA-MB-231-GFP, 4T1 (American Type Culture Collection (ATCC). Manassas, VA, USA) and MDA-MB-231-luc-D3H2LN (Caliper Life Sciences, Hopkinton, MA, USA). These cell lines are used as recommended by the supplier. Six-week-old female nude mice (nu/nu or NIH III nude) are implanted orthotopically with the human breast adenocarcinoma MDA-MB-231-luc-D3H2LN cell line (Caliper Life Sciences). In this model, orthotopically implanted tumors progress from localized disease to lymph node metastasis by 4 weeks after tumor inoculation. The tumor cells express luciferase and can be detected by noninvasive bioluminescence imaging for correlative analysis of tumor burden. All animal experiments are performed in compliance with institutional guidelines and approved by the Subcommittee on Research Animal Care (SRAC).

Prevention of metastasis: Six week-old nu/nu mice are injected in the upper right mammary fat pad with 2×106 MDA-MB-231-luc-D3H2LN cells (Caliper). Animals are used in experiments 14 days after tumor implantation.

Arrest of metastasis: Six-week-old NIH III nude mice are injected in the lower left mammary fat pad with 2×106 MDA-MB-231-luc-D3H2LN cells (Caliper). Animals are used in experiments 28 days after tumor implantation. Treatment with MN-5′pp- or MN-5′ppp-anti-miR10b and MN-5′pp- or MN-5′ppp-scr-miR involves systematic administration through the tail vein at a dose of 10 mg Fe/kg once a week over 4 weeks.

### Example 5. Design of a Template Specific RIG-I Agonist, ss-pppmiRNA-21

The Template-Specific RIG-I Agonist, ss-ppp-miRNA-21, Effectively Agonizes RIG-I and Induces Apoptosis in Melanoma Cells

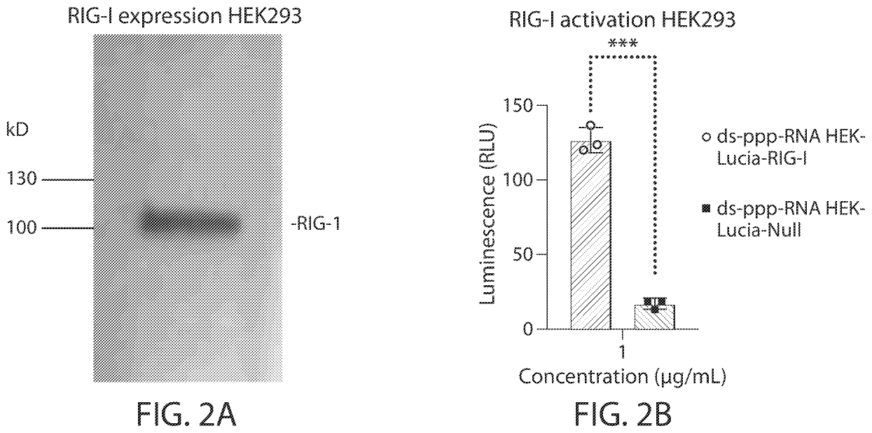

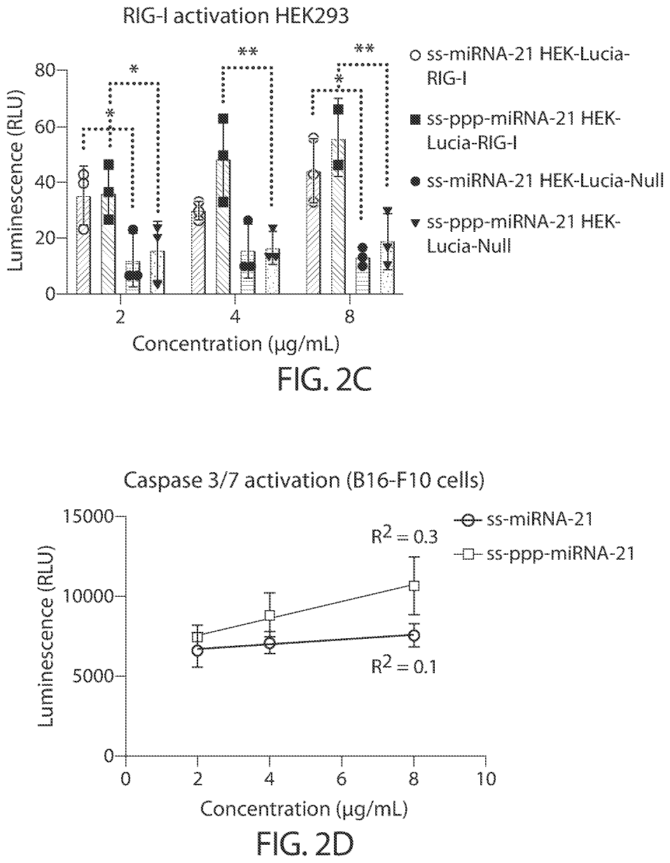

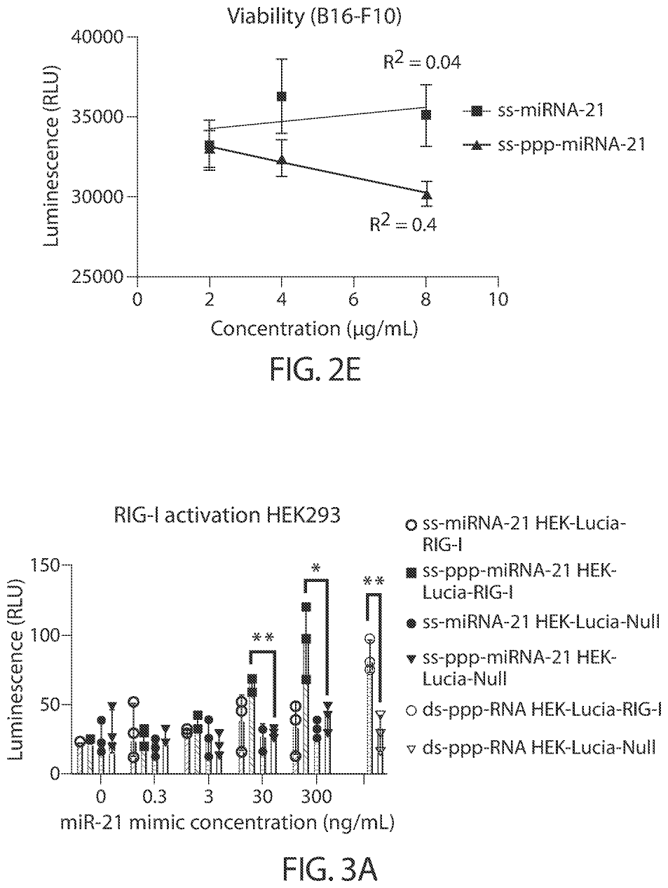

The capacity of ss-ppp-miRNA-21 to induce RIG-I activation was tested in the human RIG-I luciferase reporter cell line, HEK-Lucia™ RIG-I. The commercially available cell line stably expresses high levels of human RIG-I and the secreted Lucia luciferase reporter gene. The reporter gene is under the control of an IFN-inducible ISG54 promoter enhanced by a multimeric IFN-stimulated response elements (ISRE). HEK-Lucia™ RIG-I and HEK-Lucia™Null control cells can be used to study the role of RIG-I by monitoring IRF-induced Lucia luciferase activity. High expression of RIG-I in the cells was confirmed using Western Blot (FIG. 2A). The differential sensitivity of the HEK-Lucia™ RIG-I and HEK-Lucia™ Null control cells was validated utilizing a commercially available traditional RIG-I agonist, consisting of a 5′ triphosphate double-stranded RNA 19-mer (ds-ppp-RNA). A highly significant enhancement of luciferase activity was observed in the RIG-I overexpressing cells, as compared to the null cells (FIG. 2B).

The capacity of the template-specific RIG-I agonist, ss-ppp-miRNA-21, to activate RIG-I was evaluated. HEK-Lucia™ RIG-I and HEK-Luciam Null control cells were treated with ss-ppp-miRNA-21 as well as a single stranded oligonucleotide identical to our RIG-I agonist with the exception that the identical single stranded oligonucleotide did not incorporate a 5′-ppp. Significant RIG-I activation was observed at all three dose levels of ss-ppp-miRNA-21 tested (FIG. 2C). Given the strict requirement for the formation of an RNA duplex for RIG-I activation, these results support a template-directed mechanism of RIG-I agonism, particularly since the miR-21 complement of the single stranded RNA oligonucleotide was not exogenously supplied. Interestingly, even in the absence of a 5′-ppp, there was modest RIG-I activation (FIG. 2C).

Having established that ss-ppp-miRNA-21 can induce RIG-I in RIG-I overexpressing HEK-Lucia reporter cells, Applicant conducted experiments to determine if template-specific RIG-I agonists can mediate activation of pro-apoptotic signaling in the B16-F10 melanoma cell line. B16-F10 melanoma cells express miR-21 and have been used to study intrinsic RIG-I signaling with cell death as an endpoint (Bek et al., 2019). Caspase-3/7 activation was measured in B16-F10 melanoma cells treated with ss-ppp-miRNA-21 or the 5′-ppp-deficient ss-miRNA-21. A dose-dependent caspase 3/7 activation was observed that was more pronounced in the presence of a 5′-ppp (FIG. 2D). A dose-dependent reduction in tumor cell viability was also observed when using the ss-ppp-miRNA-21 RIG-I agonist (FIG. 2E). This reduction in tumor cell viability was significantly greater than that observed using the 5′-ppp-deficient ss-miRNA-21.

RIG-I Agonism by ss-Ppp-miRNA-21 Demonstrates Template Dependence

To further investigate the template-dependence of the observed RIG-I activation when using ss-ppp-miRNA-21, HEK-Lucia™ RIG-I cells were transiently transfected with increasing concentrations of a synthetic mature miRNA-21 mimic. A highly significant induction of RIG-I signaling by the ss-ppp-miRNA-21 agonist was observed in cells transfected with 30 and 300 ng/ml of the synthetic mature miRNA-21 mimic (FIG. 3A). Surprisingly, induction of RIG-I signaling by the ss-ppp-miRNA-21 agonist was observed in cultures of as few as 10,000 cells. The levels of activation with ss-ppp-miRNA-21 were similar to those observed with a commercially available ds-ppp-RNA positive control oligonucleotide (FIG. 3A). The 5′-ppp-deficient ss-miRNA-21 failed to cause detectable RIG-I activation (FIG. 3A). Furthermore, analysis of the dose-dependence of RIG-I activation as a function of miRNA-21 mimic concentration determined an EC50 of 83.4 ng/ml of miRNA-21 mimic when using ss-ppp-miRNA-21. By contrast, the calculated EC50 when using the 5′-ppp-deficient ss-miRNA-21 was 357.9 ng/ml (FIG. 3B).

The capacity of the template-specific ss-ppp-miRNA-21 agonist to induce an IFN-I response was evaluated in B16-F10 murine melanoma cells. Treatment with increasing concentrations of the RIG-I agonist caused a dose-dependent increase in IFN-β secretion. In cells transfected with mature miR-21 mimic, the effect was amplified, suggesting a template-specific enhancement of IFN-I stimulation by the agonist. By contrast, a commercially available ds-ppp-RNA agonist failed to stimulate IFN-β secretion (FIG. 3C).

Caspase 3/7 activation as a function of miRNA-21 mimic concentration was measured to determine consistency with the known mechanism of apoptosis induction via tumor-cell-intrinsic RIG-I signaling in B16-F10 cells transiently transfected with miRNA-21 mimic. Surprisingly, a dose-dependent increase in caspase 3/7 activation was observed, and the effect was significantly higher in cells treated with ss-ppp-miRNA-21 as compared to the 5′-ppp-deficient ss-miRNA-21, and comparable to the ds-ppp-RNA positive control (FIG. 3D).

The expression levels of RIG-I were assessed in order to determine whether, in addition to RIG-I activation, there was also evidence of RIG-I upregulation in B16-F10 cells treated with ss-ppp-miRNA-21. Low levels of RIG-I were detected in B16-F10 cells. However, in cells transfected with miR-21 and treated with ss-ppp-miRNA-21, there was dramatic upregulation of RIG-I that exceeded the levels seen with the ds-ppp-RNA positive control oligonucleotide (FIG. 3E).

One of the mechanisms of immune activation by RIG-I agonism involves the activation of the NF-κB signaling pathway. In our studies, the phosphorylation of the NF-κB subunit p65 at S536 was analyzed in order to measure NF-w B transactivation. A strong phospho-P65 reactivity in lysates from B16-F10 cells treated with ss-ppp-miRNA-21 was observed, and the strong reactivity was further amplified if the cells were also transfected with a miR-21 mimic. The increased reactivity was not associated with increased expression of p65, indicating that the increase in reactivity specifically reflected target phosphorylation (FIG. 3F). This surprising finding further supports a mechanism for effective template-dependent immune stimulation by ss-ppp-miRNA-21.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

We claim:

1. A method for treating cancer comprising administering to a subject a therapeutically effective amount of a single-stranded 5′ uncapped triphosphate or biphosphate modified RNA oligonucleotide, wherein said oligonucleotide is complementary to a miRNA, which is highly expressed in a tumor or tumor microenvironment in comparison to a non-tumor or non-tumor microenvironment.

2. A method for selectively activating RIG-I in a tumor or tumor microenvironment comprising administering to a subject a therapeutically effective amount of single-stranded Y uncapped triphosphate or biphosphate modified RNA oligonucleotide, wherein said single-stranded 5′ uncapped triphosphate or biphosphate modified RNA oligonucleotide comprises a sequence which is complementary to a miRNA expressed in the tumor or tumor microenvironment, wherein the RIG-I is selectively activated in the tumor or tumor microenvironment expressing the miRNA.3.

3. The method of claim 1 or 2, wherein the miRNA is selected from the group consisting of miR10b, miR17, miR18a, miR18b, miR19b, miR21, miR26a, miR29a, miR92a-1, miR92a-2, miR155, miR210, and miR221.

4. The method of any one of claims 1-3, wherein the single-stranded 5′ uncapped triphosphate or biphosphate modified RNA oligonucleotide forms a duplex with the miRNA.

5. The method of any one of claims 1-4, wherein the miRNA is oncogenic miRNA.

6. The method of any one of claims 1-4, wherein the miRNA is a tumor-associated miRNA.

7. The method of any one of claims 1-6, wherein the duplex is not cleaved by AGO2.

8. The method of any one of claims 1-7, wherein the duplex activates RIG-I.

9. The method of any one of claims 2-8, wherein the RIG-I activation is at least 5%, 10%, 15% or 20% greater than activation by a corresponding unmodified monophosphate RNA oligonucleotide.

10. The method of any one of claims 2-9, wherein the RIG-I activation elicits a tumor-specific immune response.

11. The method of claim 10, wherein the tumor-specific immune response comprises release of type I IFNs, DAMPs (danger-associated molecular patterns), and/or tumor antigens.

12. The method of any one of claims 1-11, wherein the method induces immunological memory against said tumor or tumor microenvironment.

13. The method of any one of claims 1-12, wherein the cancer is a solid tumor.

14. the method of claim 13, wherein the solid tumor is selected from the group consisting of sarcomas, carcinomas, and lymphomas.

15. The method of any one of claims 1-12, wherein the cancer is a non-solid tumor.

16. The method of claim 15, wherein the non-solid tumor is selected from the group consisting of leukemia, myeloma, and lymphoma.

17. The method of any one of claims 1-13, wherein the cancer is selected from the group consisting of bladder, blood, bone, brain, breast, colon, cervix, kidney, esophagus, liver, lung, thyroid, skin, ovarian, pancreatic, prostate, rectal, stomach, uterine cancer, glioblastoma, or head and neck cancer. 16. The method of any one of claims 1-15, wherein the modified RNA oligonucleotide does not comprise any other modifications.

18. The method of any one of claims 1-17, wherein the modified RNA oligonucleotide comprises at least 2 different modified RNA oligonucleotides.

19. The method of any one of claims 1-17, wherein the modified RNA oligonucleotide comprises at least 3 different modified RNA oligonucleotides.

20. The method of any one of claims 1-17, wherein the modified RNA oligonucleotide comprises at least 4 different modified RNA oligonucleotides.

21. The method of any one of claims 1-17, wherein the modified RNA oligonucleotide comprises at least 5 different modified RNA oligonucleotides.

22. The method of any one of claims 1-17, wherein the modified RNA oligonucleotide comprises up to 40 different modified RNA oligonucleotides.

23. The method of any one of claims 1-22, wherein the modified RNA oligonucleotide further comprises a 2′-fluoro (2′-F) ribose modification.

24. The method of claim 23, wherein the 2′-F ribose modification is present at the 10^{th} or 11^{th} nucleotide from the 5′-terminus of the modified RNA oligonucleotide.

25. The method of any one of claims 1-24, wherein the modified RNA oligonucleotide does not comprise a 2′-O-methyl (2′-OMe) ribose modification.

26. The method of any one of claims 1-25, wherein the modified RNA oligonucleotide does not comprise a N-6-methyladenosine (m6A) modification.

27. The method of any one of claims 1-26, wherein the modified RNA oligonucleotide does not comprise a pseudouridine (Ψ).

28. The method of any one of claims 1-27, wherein the modified RNA oligonucleotide does not comprise a N-1-methylpseudouridine (mΨ) modification.

29. The method of any one of claims 1-28, wherein the modified RNA oligonucleotide does not comprise a 5-methyl-cytidine (5mC) modification.

30. The method of any one of claims 1-29, wherein the modified RNA oligonucleotide does not comprise a 5-hydroxymethyl-cytidine (5hmC) modification.

31. The method of any one of claims 1-30, wherein the modified RNA oligonucleotide does not comprise a 5-methoxycytidine (5moC) modification.

32. The method of any one of claims 1-31, wherein the modified RNA oligonucleotide comprises a sequence which is at least 19 nucleotides in length.

33. The method of any one of claims 1-31, wherein the modified RNA oligonucleotide comprises a sequence which is between 15 and 30 nucleotides in length.

34. The method of any one of claims 1-31, wherein the modified RNA oligonucleotide comprises a sequence which is between 16 and 27 nucleotides in length.

35. The method of any one of claims 1-34, wherein the modified RNA oligonucleotide is fully complementary to the miRNA.

36. The method of any one of claims 1-35, wherein the modified RNA oligonucleotide competes with endogenous mRNA to bind the miRNA.

37. The method of any one of claims 1-35, wherein the duplex comprises between 0 and 5 mismatched base pairs.

38. The method of any one of claims 1-37, comprising administering a modified RNA oligonucleotide having the nucleic acid sequence of SEQ ID NO: 6.

39. The method of claim 38, wherein the nucleic acid of SEQ ID NO: 6 is complementary to miR-21.

40. The method of claim 38 or 39, wherein the cancer is selected from the group consisting of cancer of the breast, ovary, cervix, colon, lung, liver, brain, esophagus, prostate, pancreas, and thyroid.

41. The method of any one of claims 1-37, comprising administering a modified RNA oligonucleotide having the nucleic acid sequence of SEQ ID NO: 1.

42. The method of claim 41, wherein the nucleic acid of SEQ ID NO: 1 is complementary to miR-10b.

43. The method of claim 41 or 42, wherein the cancer is non-small cell lung cancer or cervical cancer.

44. The method of claim 43, wherein the cancer is metastatic cancer.

45. The method of any one of claims 41-44, wherein the cytosine and uracil are present at the AGO2 cleavage site.

46. The method of claim 45, wherein the metastatic cancer is localized in breast, lymph nodes, lung, bone, brain, liver, ovary, peritoneum, muscle tissue, pancreas, prostate, esophagus, colon, rectum, stomach, nasopharyngeal or skin.

47. The method of any one of claims 1-46, wherein treatment with the modified RNA oligonucleotide is a monotherapy.

48. The method of any one of claims 1-47, wherein the modified RNA oligonucleotide is administered by intravenous administration, subcutaneous, intraarterial, intramuscular, intraperitoneal, or local administration.

49. The method of any one of claims 1-48, wherein the modified RNA oligonucleotide is administered at a dose of about 0.2 mg/kg to about 200 mg/kg.

50. The method of any one of claims 1-48, wherein the modified RNA oligonucleotide is administered at a dose of about 0.2 mg/kg to about 2.0 mg/kg.

51. The method of any one of claims 1-48, wherein the modified RNA oligonucleotide is administered at a dose of about 1.0 mg/kg to about 10.0 mg/kg.

52. A method for treating cancer comprising administering to a subject a therapeutically effective amount of a magnetic nanoparticle comprising:
ferric chloride, ferrous chloride, or a combination thereof;
a dextran coating; and
a single-stranded 5′ uncapped triphosphate or biphosphate modified RNA oligonucleotide,
wherein said oligonucleotide is complementary to a miRNA, which is highly expressed in a tumor or tumor microenvironment in comparison to a non-tumor or non-tumor microenvironment.

53. The method of claim 52, wherein the magnetic nanoparticle has a non-linearity index ranging from about 6 to about 40.

54. The method of claim 52 or 53, wherein the magnetic nanoparticle has a non-linearity index ranging from about 8 to about 14.

55. The method of any one of claims 52-54, wherein the magnetic nanoparticle comprises about 0.54 g of ferric chloride and about 0.2 g of ferrous chloride.

56. The method of any one of claims 52-55, wherein the magnetic nanoparticle comprises at least 2 different modified RNA oligonucleotides.

57. The method of any one of claims 52-55, wherein the magnetic nanoparticle comprises at least 3 different modified RNA oligonucleotides.

58. The method of any one of claims 52-55, wherein the magnetic nanoparticle comprises at least 4 different modified RNA oligonucleotides.

59. The method of any one of claims 52-55, wherein the magnetic nanoparticle comprises at least 5 different modified RNA oligonucleotides.

60. The method of any one of claims 52-55, wherein the magnetic nanoparticle comprises between up to 40 different modified RNA oligonucleotides.

61. The method of any one of claims 52-60, wherein the miRNA is selected from the group consisting of miR10b, miR17, miR18a, miR18b, miR19b, miR21, miR26a, miR29a, miR92a-1, miR92a-2, miR155, miR210, and miR221.

62. The method of any one of claims 52-61, wherein the miRNA is oncogenic miRNA.

63. The method of any one of claims 52-61, wherein the miRNA is a tumor-associated miRNA.

64. The method of any one of claims 1-63, further comprising administering supportive or adjunctive therapy.

65. The method of claim 64, wherein the adjunctive therapy comprises radiotherapy, cryotherapy, and ultrasound therapy.

66. The method of claim 64 or 65, wherein the method comprises administering additional therapeutic agents.

67. The method of any one of claims 64-66, wherein the additional therapeutic agent comprises a miRNA.

68. The method of any one of claims 64-66, wherein the miRNA of claim 67 is complementary to the modified RNA oligonucleotide.

69. The method of claim 66, wherein the additional therapeutic agent is selected from the group consisting of a targeted therapy, chemotherapeutic agent, immunotherapeutic agent, an immunogenic cell death inducer (ICDi), and an siRNA therapy.

70. The method of claim 69, wherein the method further comprises surgery.

71. The method of claim 69, wherein the chemotherapeutic agent is selected from the group consisting of cyclophosphamide, mechlorethamine, chlorambucil, melphalan, daunorubicin, doxorubicin, epirubicin, idarubicin, mitoxantrone, valrubicin, paclitaxel, docetaxel, etoposide, teniposide, tafluposide, azacitidine, azathioprine, capecitabine, cytarabine, doxifluridine, fluorouracil, gemcitabine, mercaptopurine, methotrexate, tioguanine, bleomycin, carboplatin, cisplatin, oxaliplatin, all-trans retinoic acid, vinblastine, vincristine, vindesine, vinorelbine, and bevacizumab.

72. The method of claim 69, wherein the targeted therapy is selected from the group consisting of trastuzumab, gilotrif, proleukin, alectinib, campath, atezolizumab, avelumab, axitinib, belimumab, belinostat, bevacizumab, velcade, canakinumab, ceritinib, cetuximab, crizotinib, dabrafenib, daratumumab, dasatinib, denosumab, elotuzumab, enasidenib, erlotinib, gefitinib, ibrutinib, zydelig, imatinib, lenvatinib, midostaurin, necitumumab, niraparib, obinutuzumab, osimertinib, panitumumab, regorafenib, rituximab, ruxolitinib, sorafenib, tocilizumab, and trastuzumab.

73. The method of claim 69, wherein the immunotherapeutic agent is an immune checkpoint inhibitor.

74. The method of claim 73, wherein the immune checkpoint inhibitor is selected from the group consisting of pembrolizumab (Keytruda®), nivolumab (Opdivo®), atezolizumab (Tecentriq®), ipilimumab (Yervoy®), avelumab (Bavencio®) and durvalumab (Imfinzi®).

75. The method of any of the claims 64-74, wherein the adjunctive therapy induces expression of the miRNA.

76. The method of any of the claims 66-73, wherein the additional therapeutic agent induces expression of the miRNA.

77. The method of claim 73, wherein the ICDi is selected from the group consisting of Daunorubicin, Docetaxel, Doxorubicin, Mitoxanthrone, Oxaliplatin, and Paclitaxel.

78. The method of claim 73, wherein the siRNA therapy targets PD-L1, CTLA-4, TGF-β, and/or VEGF.

79. The method of anyone of claims of 64-78, wherein the supportive or adjunctive therapy is administered prior, concurrently, or after administration of the modified RNA oligonucleotide.

80. A single-stranded 5′ uncapped triphosphate or biphosphate modified RNA oligonucleotide, wherein said oligonucleotide is complementary to a miRNA, which is highly expressed in tumor tissue in comparison to non-tumor tissue.

81. The modified RNA oligonucleotide of claim 80, wherein the miRNA is selected from the group consisting of miR10b, miR17, miR18a, miR18b, miR19b, miR21, miR26a, miR29a, miR92a-1, miR92a-2, miR155, miR210, and miR221.

82. The modified RNA oligonucleotide of claim 80 or 81, wherein the modified RNA oligonucleotide is capable of forming a duplex with the said miRNA.

83. The modified RNA oligonucleotide of any one of claims 80-82, wherein the duplex is not cleaved by AGO2.

84. The modified RNA oligonucleotide of any one of claims 80-83, wherein the duplex activates RIG-I.

85. The modified RNA oligonucleotide of claim 84, wherein the RIG-I activation is at least 5%, 10%, 15% or 20% greater than activation by a corresponding unmodified monophosphate RNA oligonucleotide.

86. The modified RNA oligonucleotide of claim 84 or 85, wherein the RIG-I activation elicits a tumor-specific immune response.

87. The modified RNA oligonucleotide of claim 86, wherein the tumor-specific immune response comprises release of type I IFNs, DAMPs (danger-associated molecular patterns), and/or tumor antigens.

88. The modified RNA oligonucleotide of any one of claims 80-87, wherein the modified RNA oligonucleotide does not comprise any other modifications.

89. The modified RNA oligonucleotide of any one of claims 80-88, wherein the modified RNA oligonucleotide further comprises a 2′-fluoro (2′-F) ribose modification.

90. The modified RNA oligonucleotide of any one of claims 80-89, wherein the modified RNA oligonucleotide does not comprise a 2′-O-methyl (2′-OMe) ribose modification.

91. The modified RNA oligonucleotide of any one of claims 80-90, wherein the modified RNA oligonucleotide does not comprise a N-6-methyladenosine (m6A) modification.

92. The modified RNA oligonucleotide of any one of claims 80-91, wherein the modified RNA oligonucleotide does not comprise a pseudouridine (Ψ).

93. The modified RNA oligonucleotide of any one of claims 80-92, wherein the modified RNA oligonucleotide does not comprise a N-1-methylpseudouridine (mΨ) modification.

94. The modified RNA oligonucleotide of any one of claims 80-92, wherein the modified RNA oligonucleotide does not comprise a 5-methyl-cytidine (5mC) modification.

95. The modified RNA oligonucleotide of any one of claims 80-94, wherein the modified RNA oligonucleotide does not comprise a 5-hydroxymethyl-cytidine (5hmC) modification.

96. the modified RNA oligonucleotide of any one of claims 80-95, wherein the modified RNA oligonucleotide does not comprise a 5-methoxycytidine (5moC) modification.

97. The modified RNA oligonucleotide of any one of claims 80-96, wherein the modified RNA oligonucleotide is fully complementary to the miRNA.

98. The modified RNA oligonucleotide of any one of claims 80-97, wherein the modified RNA oligonucleotide competes with endogenous mRNA to bind the miRNA.

99. The modified RNA oligonucleotide of any one of claims 82-98, wherein the duplex comprises between 0 and 5 mismatched base pairs.

100. The modified RNA oligonucleotide of any one of claims 80-99, wherein the modified RNA oligonucleotide comprises a nucleic acid sequence of any one of SEQ ID NOs: 1-13.

101. The modified RNA oligonucleotide of any one of claims 80-100, wherein the modified oligonucleotide is further linked to a nanoparticle.

102. The modified RNA oligonucleotide of claim 101, wherein the nanoparticle is a magnetic nanoparticle.

103. The modified RNA oligonucleotide of claim 102, wherein the magnetic nanoparticle is coated with a polymer coating.

104. The modified RNA oligonucleotide of claim 103, wherein the polymer coating is dextran.

105. The modified RNA oligonucleotide of any one of claims 102-104, wherein the magnetic nanoparticle comprises iron oxide; and a dextran coating functionalized with one or more amine groups, wherein the number of the one or more amine groups ranges from about 5 to about 1000.

106. The modified RNA oligonucleotide of any one of claims 102-105, wherein the iron content of the magnetic nanoparticle comprises about 50% weight (wt) to about 100% wt of iron (III) and about 0% wt to about 50% wt of iron (II).

107. The modified RNA oligonucleotide of any one of claims 102-106, wherein the magnetic nanoparticle comprises from about 5 to about 150 amino groups.

108. The modified RNA oligonucleotide of any one of claims 102-107, wherein the magnetic nanoparticle comprises one or more such modified RNA oligonucleotides.

109. A magnetic nanoparticle comprising:
ferric chloride, ferrous chloride, or a combination thereof;
a dextran coating; and
a single-stranded 5′ uncapped triphosphate or biphosphate modified RNA oligonucleotide, wherein said oligonucleotide is complementary to a miRNA, which is highly expressed in a tumor or tumor microenvironment in comparison to a non-tumor or non-tumor microenvironment.

110. The magnetic nanoparticle of claim 109, wherein the magnetic nanoparticle has a non-linearity index ranging from about 6 to about 40.

111. The magnetic nanoparticle of claim 109 or 110, wherein the magnetic nanoparticle has a non-linearity index ranging from about 8 to about 14.

112. The magnetic nanoparticle of any one of claims 109-111, wherein the magnetic nanoparticle comprises about 0.54 g of ferric chloride and about 0.2 g of ferrous chloride.

113. The magnetic nanoparticle of any one of claims 109-112, wherein the miRNA is selected from the group consisting of miR10b, miR17, miR18a, miR18b, miR19b, miR21, miR26a, miR29a, miR92a-1, miR92a-2, miR155, miR210, and miR221.

114. The magnetic nanoparticle of any one of claims 109-113, wherein the miRNA is oncogenic miRNA.

115. The magnetic nanoparticle of any one of claims 109-113, wherein the miRNA is a tumor-associated miRNA.

116. The magnetic nanoparticle of any one of claims 109-115, wherein the magnetic nanoparticle comprises two or more modified RNA oligonucleotides.

117. The magnetic nanoparticle of claim 116, wherein the two or more modified RNA oligonucleotides are complementary to different miRNAs.

118. The magnetic nanoparticle of claim 116, wherein the two or more modified RNA oligonucleotides are complementary to the same miRNA.

119. A pharmaceutical composition comprising the modified RNA oligonucleotide of any one of claims 80-108 or the magnetic nanoparticle of any one of claims 109-118.

120. The pharmaceutical composition of claim 119 that further comprises a delivery agent.

121. The pharmaceutical composition of claim 120, wherein the delivery agent is selected from the group consisting of a micelle, lipid nanoparticle (LNP), spherical nucleic acid (SNA), extracellular vesicle, synthetic vesicle, exosome, lipidoid, liposome, and lipoplex.

122. The pharmaceutical composition of claim 121, wherein the liposome is formed from a lipid bilayer.

123. The pharmaceutical composition of claim 122, wherein the lipid bilayer comprises one or more phospholipids selected from the group consisting of phosphate lipids, phosphoglycerol lipids, phosphocholine lipids, and phosphoethanolamine lipids.

124. The pharmaceutical composition of claim 123, wherein the phospholipids are PEGylated.

125. The pharmaceutical composition of claim 121, wherein the delivery agent is a liposome or lipid nanoparticle.

126. The pharmaceutical composition of claim 125, wherein the liposome or lipid nanoparticle further delivers an additional therapeutic agent.

127. The pharmaceutical composition of claim 126, wherein the additional therapeutic agent is an ICDi (e.g., Daunorubicin, Docetaxel, Doxorubicin, Mitoxanthrone, Oxaliplatin, and Paclitaxel).

128. The pharmaceutical composition of claim 126, wherein the additional therapeutic agent is an siRNA (e.g., an siRNA targeting a gene associated with cancer).

129. The pharmaceutical composition of claim 126, wherein the additional therapeutic agent is a chemotherapeutic agent.

130. The pharmaceutical composition of any one of claims 119-129, comprising at least one additional modified RNA oligonucleotide.

131. The pharmaceutical composition of any one of claims 119-130, wherein the modified RNA oligonucleotide is administered at a dose of about 0.2 mg/kg to about 200 mg/kg.

132. The pharmaceutical composition of any one of claims 119-130, wherein the modified RNA oligonucleotide is administered at a dose of about 0.2 mg/kg to about 2.0 mg/kg.

133. The pharmaceutical composition of any one of claims 119-130, wherein the modified RNA oligonucleotide is administered at a dose of about 1.0 mg/kg to about 10.0 mg/kg.
